(19) United States
(12) Patent Application Publication
(10) Pub. No.: US 2007/0088001 A1
(43) Pub. Date: Apr. 19, 2007

(54) Silinane compounds as cysteine protease inhibitors

(76) Inventors: John O. Link, San Francisco, CA (US); Michael Graupe, Pacifica, CA (US)

Correspondence Address: BERLEX BIOSCIENCES;PATENT DEPARTMENT, 2600 HILLTOP DRIVE, P.O. BOX 4099, RICHMOND, CA, 94804-0099, US

(21) Appl. No.: 10/587,867

(22) PCT Filed: Jan. 31, 2005
(86) PCT No.: PCT/US05/02773

§ 371 Date: Dec. 21, 2006

Related U.S. Application Data
(60) Provisional application No. 60/540,581, filed on Jan. 30, 2004.
(60) Provisional application No. 60/547,498, filed on Feb. 24, 2004.

(51) Int. Cl. A61K 31/695; C07F 7/08

(52) U.S. Cl. 514/63; 544/69

(57) ABSTRACT

The present invention is directed to compounds that are inhibitors of cysteine proteases, in particular, cathepsins B, K, L, F, and S and are therefore useful in treating diseases mediated by these proteases. The present invention is also directed to pharmaceutical compositions comprising these compounds and processes for preparing them. The present invention is also directed to the use of these inhibitors in combination with a therapy that causes a deleterious immune response in patients receiving the therapy.

### FIELD OF THE INVENTION

The present invention is directed to compounds that are inhibitors of cysteine proteases, in particular, cathepsins B, K, L, F, and S and are therefore useful in treating diseases mediated by these proteases. The present invention is also directed to pharmaceutical compositions comprising these compounds and processes for preparing them. The present invention is also directed to the use of these inhibitors in combination with a therapy that causes a deleterious immune response in patients receiving the therapy.

### STATE OF THE ART

Cysteine proteases represent a class of peptidases characterized by the presence of a cysteine residue in the catalytic site of the enzyme. Cysteine proteases are associated with the normal degradation and processing of proteins. The aberrant activity of cysteine proteases, e.g., as a result of increased expression or enhanced activation, however, may have pathological consequences. In this regard, certain cysteine proteases are associated with a number of disease states, including arthritis, muscular dystrophy, inflammation, tumor invasion, glomerulonephritis, malaria, periodontal disease, metachromatic leukodystrophy, and others. For example, increased cathepsin B levels and redistribution of the enzyme are found in tumors; thus, suggesting a role for the enzyme in tumor invasion and metastasis. In addition, aberrant cathepsin B activity is implicated in such disease states as rheumatoid arthritis, osteoarthritis, pneumocystis carinii, acute pancreatitis, inflammatory airway disease, and bone and joint disorders.

The prominent expression of cathepsin K in osteoclasts and osteoclast-related multinucleated cells and its high collagenolytic activity suggest that the enzyme is involved in osteoclast-mediated bone resorption and hence in bone abnormalities such as occurs in osteoporosis. In addition, cathepsin K expression in the lung and its elastinolytic activity suggest that the enzyme plays a role in pulmonary disorders as well.

Cathepsin L is implicated in normal lysosomal proteolysis as well as several disease states, including, but not limited to, metastasis of melanomas. Cathepsin S is implicated in Alzheimer's disease and certain autoimmune disorders including, but not limited to juvenile onset diabetes, multiple sclerosis, pemphigus vulgaris, Graves' disease, myasthenia gravis, systemic lupus erythemotasus, rheumatoid arthritis, and Hashimoto's thyroiditis. In addition, cathepsin S is implicated in: allergic disorders including, but not limited to asthma and allogeneic immune reponses including, but not limited to, rejection of organ transplants or tissue grafts.

Another cysteine protease, Cathepsin F, has been found in macrophages and is involved in antigen processing. It is believed that Cathepsin F in stimulated lung macrophages and possibly other antigen presenting cells could play a role in airway inflammation (see G. P. Shi et al, J. Exp. Med. 2000, 191, 1177)

In view of the number of diseases wherein it is recognized that an increase in cysteine protease activity contributes to the pathology and/or symptomatology of the disease, molecules which inhibit the activity of this class of enzymes, in particular molecules which inhibitor cathepsins B, K, L, F, and/or S, will therefore be useful as therapeutic agents.

### DETAILED DESCRIPTION

In a first aspect, this invention is directed to a compound of Formula (I): 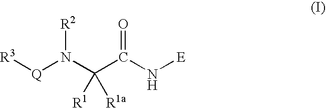
wherein:

Q is —CO—, —SO₂—, —OCO—, —NR⁴CO—, —NR⁴SO₂—, or —CHR— where R is haloalkyl and R⁴ is hydrogen, alkyl, hydroxyalkyl, alkoxyalkyl, or aralkyl;

E is:
(i) —C(R⁵)(R⁶)X¹ where X¹ is —C(R⁷)(R⁸)R¹⁰, —CH═CHS(O)₂R¹⁰, —C(R⁷)(R⁸)C(R⁷)(R⁸)OR¹⁰, —C(R⁷)(R⁸)CH₂OR¹⁰, —C(R⁷)(R⁸)CH₂N(R¹¹)SO₂R¹⁰, —C(R⁷)(R⁸)C(O)N(R¹¹)(CH₂)₂OR¹¹, —C(R⁷)(R⁸)C(O)NR¹⁰R¹¹ or —C(R⁷)(R⁸)C(O)N(R¹¹)(CH₂)₂NR¹⁰R¹¹;
(ii) —C(R^{5a})(R^{6a})CN;
   where:

R⁵ and R^{5a} are independently hydrogen or alkyl;

R⁶ and R^{6a} are independently selected from the group consisting of hydrogen, alkyl, haloalkyl, cycloalkyl, cycloalkylalkyl, aryl, aralkyl, heteroaryl, heteroaralkyl, heterocycloalkyl, heterocycloalkylalkyl, -alkylene-X²—R¹² (where X² is —O—, —NR¹³—, —S(O)ₙ₁—, —CONR¹³—, —NR¹³CO—, —NR¹³C(O)O—, —NR¹³CONR¹³—, —OCONR¹³—, —NR¹³SO₂—, —SO₂NR¹³—, —NR¹³SO₂NR¹³—, —CO—, or —OC(O)— where n1 is 0-2 and each R¹³ is hydrogen or alkyl) and R¹² hydrogen, alkyl, haloalkyl, cycloalkyl, cycloalkylalkyl, heterocycloalkyl, heterocycloalkylalkyl, aryl, aralkyl, heteroaryl, or heteroaralkyl wherein the aromatic or alicyclic ring in R⁶ and R^{6a} is optionally substituted with one, two, or three R^{a} independently selected from alkyl, haloalkyl, alkoxy, hydroxy, haloalkoxy, halo, carboxy, alkoxycarbonyl, amino, monsubstituted amino, disubstituted amino, nitro, aryloxy, benzyloxy, acyl, alkylsulfonyl, or arylsulfonyl where the aromatic or alicyclic ring in R^{a} is optionally substituted with one or two substituents independently selected from alkyl, halo, alkoxy, haloalkyl, haloalkoxy, hydroxy, amino, alkylamino, dialkylamino, carboxy, or alkoxycarbonyl; or

R⁵ and R⁶ and R^{5a} and R^{6a} taken together with the carbon atom to which both R⁵ and R⁶ and R^{5a} and R^{6a} are attached form (i) cycloalkylene optionally substituted with one or two R^{b} independently selected from alkyl, halo, alkylamino, dialkylamino, aryl, aralkyl, cycloalkyl, cycloalkylalkyl, heteroaryl, heteroaralkyl, alkoxycarbonyl, or aryloxycarbonyl or (ii) heterocycloalkylene optionally substituted with one to four alkyl or one or two R^{c} independently selected from alkyl, haloalkyl, hydroxy, hydroxyalkyl, alkoxyalkyl, alkoxyalkyloxyalkyl, aryloxyalkyl, heteroaryloxyalkyl, aminoalkyl, acyl, aryl, aralkyl, heteroaryl, heteroaralkyl, heterocycloalkyl, heterocycloalkylalkyl, cycloalkyl, cycloalkylalkyl, —S(O)ₙ₂R¹⁴, -alkylene-S(O)ₙ₂—R¹⁵, —COOR¹⁶, -alkylene-COOR¹⁷, —CONR¹⁸R¹⁹, or -alkylene-CONR²⁰R²¹ (where n2 is 0-2 and R¹⁴-R¹⁷, R¹⁸ and R²⁰ are independently hydrogen, alkyl, haloalkyl, aryl, aralkyl, heteroaryl, heteroaralkyl, cycloalkyl, cycloalkylalkyl, or heterocycloalkyl and R¹⁹ and R²¹ are independently hydrogen or alkyl) wherein the aromatic or alicyclic ring in the groups attached to cycloalkylene or heterocycloalkylene is optionally substituted with one, two, or three substituents independently selected from alkyl, haloalkyl, cycloalkyl, cycloalkylalkyl, benzyl, alkoxy, hydroxy, haloalkoxy, halo, carboxy, alkoxycarbonyl, amino, monsubstituted amino, disubstituted amino, or acyl;

R⁷ is hydrogen or alkyl;

R⁸ is hydroxy; or

R⁷ and R⁸ together form oxo;

R¹⁰ is alkyl, aryl, aralkyl, heteroaryl, heteroaralkyl, cycloalkyl, cycloalkylalkyl, heterocycloalkyl, or heterocycloalkylalkyl wherein the aromatic or alicyclic ring in R¹⁰ is optionally substituted with one, two, or three R^{d} independently selected from alkyl, haloalkyl, alkoxy, alkoxyalkyl, cycloalkyl, hydroxy, haloalkoxy, halo, carboxy, alkoxycarbonyl, aminosulfonyl, alkylsulfonyl, arylsulfonyl, heteroarylsulfonyl, aryl, aralkyl, heteroaryl, amino, monsubstituted amino, disubstituted amino, carbamoyl, or acyl and wherein the aromatic or alicyclic ring in R¹ is optionally substituted with one, two, or three substitutents independently selected from alkyl, haloalkyl, alkoxy, haloalkoxy, halo, hydroxy, carboxy, alkoxycarbonyl, amino, alkylamino, or dialkylamino; and

R¹¹ is hydrogen or alkyl; or

(iii) a group of formula (a): 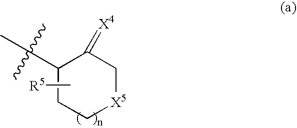
where:

n is 0, 1, or 2;

X⁴ is selected from —NR²²—, —S—, or —O— where R²² is hydrogen, alkyl, or alkoxy; and

X⁵ is —O—, —S—, —SO₂—, or —NR²³ where R²³ is selected from hydrogen, alkyl, haloalkyl, hydroxyalkyl, alkoxyalkyl, aryloxyalkyl, heteroaryloxyalkyl, aminoalkyl, acyl, aryl, aralkyl, heteroaryl, heteroaralkyl, cycloalkyl, cycloalkylalkyl, —S(O)₂R²⁴, -alkylene-S(O)ₙ₃—R²⁵, —COOR²⁶, -alkylene-COOR²⁷, —CONR²⁸R²⁹, or -alkylene-CONR³⁰R³¹ (where n3 is 0-2 and R²⁴-R²⁷, R²⁸ and R³⁰ are independently hydrogen, alkyl, haloalkyl, aryl, aralkyl, heteroaryl, heteroaralkyl, cycloalkyl, cycloalkylalkyl, heterocycloalkyl, or heterocycloalkylalkyl and R²⁹ and R³¹ are independently hydrogen or alkyl) where the aromatic or alicyclic ring in R²³ is optionally substituted with one, two, or three substituents independently selected from alkyl, haloalkyl, alkoxy, haloalkoxy, halo, hydroxy, amino, alkylamino, dialkylamino, carboxy, or alkoxycarbonyl and one substitutent selected from aryl, aralkyl, heteroaryl, or heteroaralkyl; and

R⁵ is as defined above;

R¹ is hydrogen or alkyl;

R^{1a} is 1,1-dialkylsilinan-4-ylalkylene or -(alkylene)-SiR³²R³³R³⁴ where R³² is alkyl, R³³ is alkyl, and R³⁴ is alkyl, alkenyl, cycloalkylalkyl, aryl, aralkyl, heteroaralkyl, or heterocycloalkylalkyl or R³³ and R³⁴ together with Si form a heterocycloalkylene ring containing the Si atom and 3 to 7 carbon ring atoms wherein one or two carbon ring atoms are optionally independently replaced with —NH—, —O—, —S—, —SO—, —SO₂—, —CO—, —CONH—, or —SO₂NH— and wherein the aralkyl, heteroaralkyl, heterocycloalkyl, or heterocycloalkylene ring in R^{1a} is optionally substituted on the ring with one, two, or three R^{e} independently selected from alkyl, haloalkyl, alkoxy, hydroxy, haloalkoxy, halo, carboxy, alkoxycarbonyl, amino, monsubstituted amino, disubstituted amino, nitro, aryloxy, benzyloxy, acyl, alkylsulfonyl, or arylsulfonyl and further wherein the aromatic or alicyclic ring in R^{e} is optionally substituted with one or two substituents independently selected from alkyl, halo, alkoxy, haloalkyl, haloalkoxy, hydroxy, amino, alkylamino, dialkylamino, carboxy, or alkoxycarbonyl;

R² is hydrogen or alkyl;

R³ is alkyl, haloalkyl, cycloalkyl, cycloalkylalkyl, aryl, aralkyl, heteroaryl, heteroaralkyl, heterocycloalkyl, heterocycloalkylalkyl, or -alkylene-X⁶—R³⁵ [wherein X⁶ is —NR³⁶—, —O—, —S(O)ₙ₄—, —CO—, —COO—, —OCO—, —NR³⁶CO—, —CONR³⁶—, —NR³⁶SO₂—, —SO₂NR³⁶—, —NR³⁶COO—, —OCONR³⁶—, —NR³⁶CONR³⁷—, or —NR³⁶SO₂NR³⁷— (where each R³⁶ and R³⁷ is independently hydrogen, alkyl, or acyl and n4 is 0-2) and R³⁵ is hydrogen, alkyl, haloalkyl, cycloalkyl, cycloalkylalkyl, heterocycloalkyl, heterocycloalkylalkyl, aryl, aralkyl, heteroaryl, or heteroaralkyl] wherein the alkylene chain in R³ is optionally substituted with one to four halo atoms and the aromatic and alicyclic rings in R³ are optionally substituted by one, two, or three R^{f} independently selected from alkyl, aminoalkyl, halo, hydroxy, alkoxy, haloalkyl, haloalkoxy, oxo, cyano, nitro, acyl, acyloxy, aryl, aralkyl, heteroaryl, heteroaralkyl, cycloalkyl, cycloalkylalkyl, heterocycloalkyl, heterocycloalkylalkyl, aryloxy, benzyloxy, carboxy, alkoxycarbonyl, aryloxycarbonyl, carbamoyl, alkylthio, alkylsulfinyl, alkylsulfonyl, arylthio, arylsulfonyl, arylsulfinyl, alkoxycarbonylamino, aryloxycarbonylamino, alkylcarbamoyloxy, arylcarbamoyloxy, alkylsulfonylamino, arylsulfonylamino, aminosulfonyl, alkylaminosulfonyl, dialkylaminosulfonyl, arylaminosulfonyl, aralkylaminosulfonyl, aminocarbonyl, arylaminocarbonyl, aralkylaminocarbonyl, amino, monosubsituted or disubstituted amino, and further wherein the aromatic and alicyclic rings in R^{f} are optionally substituted with one, two, or three R^{g} wherein R^{g} is independently selected from alkyl, halo, haloalkyl, haloalkoxy, hydroxy, nitro, cyano, hydroxyalkyl, alkoxy, alkoxyalkyl, aminoalkyl, alkylthio, alkylsulfonyl, amino, monosubstituted amino, dialkylamino, aryl, heteroaryl, cycloalkyl, carboxy, carboxamido, or alkoxycarbonyl; or

a pharmaceutically acceptable salts thereof.

Preferably, R¹¹ is alkyl when E is —C(R⁷)(R⁸)C(O)NR¹⁰R¹¹.

In a second aspect, this invention is directed to a method for treating a disease in an animal mediated by cysteine proteases, in particular cathepsin S, which method comprises administering to the animal a therapeutically effective amount of a compound of Formula (I): 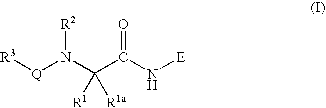
where:

Q is —CO—, —SO₂—, —OCO—, —NR⁴CO—, —NR⁴SO₂—, or —CHR— where R is haloalkyl and R⁴ is hydrogen, alkyl, hydroxyalkyl, alkoxyalkyl, or aralkyl;

E is:
(i) —C(R⁵)(R⁶)X¹ where X¹ is —CHO, —C(R⁷)(R⁸)CF₃, —C(R⁷)(R⁸)CF₂CF₂R⁹, —C(R⁷)(R⁸)R¹⁰, —CH═CHS(O)₂R¹⁰, —C(R⁷)(R⁸)C(R⁷)(R⁸)OR¹⁰, —C(R⁷)(R⁸)CH₂OR¹⁰, —C(R⁷)(R⁸)C(R⁷)(R⁸)R¹⁰, —C(R⁷)(R⁸)CH₂N(R¹¹)SO₂R¹⁰, —C(R⁷)(R⁸)CF₂C(O)NR¹⁰R¹¹, —C(R⁷)(R⁸)C(O)NR¹⁰R¹¹, —C(R⁷)(R⁸)C(O)N(R¹¹)(CH₂)₂OR¹¹, or —C(R⁷)(R⁸)C(O)N(R¹¹)(CH₂)₂NR¹⁰R¹¹;
(ii) —C(R^{5a})(R^{6a})CN;
   where:

R⁵ and R^{5a} are independently hydrogen or alkyl;

R⁶ and R^{6a} are independently selected from the group consisting of hydrogen, alkyl, haloalkyl, cycloalkyl, cycloalkylalkyl, aryl, aralkyl, heteroaryl, heteroaralkyl, heterocycloalkyl, heterocycloalkylalkyl, -alkylene-X²—R¹² (where X² is —O—, —NR¹³—, —S(O)ₙ₁—, —CONR¹³—, —NR¹³CO—, —NR¹³C(O)O—, —NR¹³CONR¹³—, —OCONR¹³—, —NR¹³SO₂— —SO₂NR³—, —NR¹³SO₂NR¹³—, —CO—, or —OC(O)— where n1 is 0-2 and each R¹³ is hydrogen or alkyl) and R¹² hydrogen, alkyl, haloalkyl, cycloalkyl, cycloalkylalkyl, heterocycloalkyl, heterocycloalkylalkyl, aryl, aralkyl, heteroaryl, or heteroaralkyl wherein the aromatic or alicyclic ring in R⁶ and R^{6a} is optionally substituted with one, two, or three R^{a} independently selected from alkyl, haloalkyl, alkoxy, hydroxy, haloalkoxy, halo, carboxy, alkoxycarbonyl, amino, monsubstituted amino, disubstituted amino, nitro, aryloxy, benzyloxy, acyl, alkylsulfonyl, or arylsulfonyl where the aromatic or alicyclic ring in R¹ is optionally substituted with one or two substituents independently selected from alkyl, halo, alkoxy, haloalkyl, haloalkoxy, hydroxy, amino, alkylamino, dialkylamino, carboxy, or alkoxycarbonyl; or

R⁵ and R⁶ and R^{5a} and R^{6a} taken together with the carbon atom to which both R⁵ and R⁶ and R^{5a} and R^{6a} are attached form (i) cycloalkylene optionally substituted with one or two R^{b} independently selected from alkyl, halo, alkylamino, dialkylamino, aryl, aralkyl, cycloalkyl, cycloalkylalkyl, heteroaryl, heteroaralkyl, alkoxycarbonyl, or aryloxycarbonyl or (ii) heterocycloalkylene optionally substituted with one to four alkyl or one or two R^{c} independently selected from alkyl, haloalkyl, hydroxy, hydroxyalkyl, alkoxyalkyl, alkoxyalkyloxyalkyl, aryloxyalkyl, heteroaryloxyalkyl, aminoalkyl, acyl, aryl, aralkyl, heteroaryl, heteroaralkyl, heterocycloalkyl, heterocycloalkylalkyl, cycloalkyl, cycloalkylalkyl, —S(O)ₙ₂R¹⁴, -alkylene-S(O)ₙ₂—R¹⁵, —COOR⁶, -alkylene-COOR¹⁷, —CONR¹⁸R¹⁹, or -alkylene-CONR²⁰R²¹ (where n2 is 0-2 and R¹⁴-R¹⁷, R¹⁸ and R²⁰ are independently hydrogen, alkyl, haloalkyl, aryl, aralkyl, heteroaryl, heteroaralkyl, cycloalkyl, cycloalkylalkyl, or heterocycloalkyl and R¹⁹ and R²¹ are independently hydrogen or alkyl) wherein the aromatic or alicyclic ring in the groups attached to cycloalkylene or heterocycloalkylene is optionally substituted with one, two, or three substituents independently selected from alkyl, haloalkyl, cycloalkyl, cycloalkylalkyl, benzyl, alkoxy, hydroxy, haloalkoxy, halo, carboxy, alkoxycarbonyl, amino, monsubstituted amino, disubstituted amino, or acyl;

R⁷ is hydrogen or alkyl;

R⁸ is hydroxy; or

R⁷ and R⁸ together form oxo;

R⁹ is hydrogen, halo, alkyl, aralkyl or heteroaralkyl;

R¹⁰ is alkyl, aryl, aralkyl, heteroaryl, heteroaralkyl, cycloalkyl, cycloalkylalkyl, heterocycloalkyl, or heterocycloalkylalkyl wherein the aromatic or alicyclic ring in R¹⁰ is optionally substituted with one, two, or three R^{d} independently selected from alkyl, haloalkyl, alkoxy, alkoxyalkyl, cycloalkyl, hydroxy, haloalkoxy, halo, carboxy, alkoxycarbonyl, aminosulfonyl, alkylsulfonyl, arylsulfonyl, heteroarylsulfonyl, aryl, aralkyl, heteroaryl, amino, monsubstituted amino, disubstituted amino, carbamoyl, or acyl and wherein the aromatic or alicyclic ring in R^{d} is optionally substituted with one, two, or three substitutents independently selected from alkyl, haloalkyl, alkoxy, haloalkoxy, halo, hydroxy, carboxy, alkoxycarbonyl, amino, alkylamino, or dialkylamino; and

R¹¹ is hydrogen or alkyl; or

(iii) a group of formula (a): 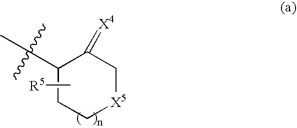
where:

n is 0, 1, or 2;

X⁴ is selected from —NR²²—, —S—, or —O— where R²² is hydrogen, alkyl, or alkoxy; and

X⁵ is —O—, —S—, —SO₂—, or —NR²³— where R²³ is selected from hydrogen, alkyl, haloalkyl, hydroxyalkyl, alkoxyalkyl, aryloxyalkyl, heteroaryloxyalkyl, aminoalkyl, acyl, aryl, aralkyl, heteroaryl, heteroaralkyl, cycloalkyl, cycloalkylalkyl, —S(O)₂R²⁴, -alkylene-S(O)ₙ₃—R²⁵, —COOR²⁶, -alkylene-COOR²⁷, —CONR²⁸R²⁹, or -alkylene-CONR³⁰R³¹ (where n3 is 0-2 and R²⁴—R²⁷, R²⁸ and R³⁰ are independently hydrogen, alkyl, haloalkyl, aryl, aralkyl, heteroaryl, heteroaralkyl, cycloalkyl, cycloalkylalkyl, heterocycloalkyl, or heterocycloalkylalkyl and R²⁹ and R³¹ are independently hydrogen or alkyl) where the aromatic or alicyclic ring in R²³ is optionally substituted with one, two, or three substituents independently selected from alkyl, haloalkyl, alkoxy, haloalkoxy, halo, hydroxy, amino, alkylamino, dialkylamino, carboxy, or alkoxycarbonyl and one substitutent selected from aryl, aralkyl, heteroaryl, or heteroaralkyl; and

R⁵ is as defined above;

R¹ is hydrogen or alkyl;

R^{1a} is 1,1-dialkylsilinan-4-ylalkylene or -(alkylene)-SiR³²R³³R³⁴ where R³² is alkyl, R³³ is alkyl, and R³⁴ is alkyl, alkenyl, cycloalkylalkyl, aryl, aralkyl, heteroaralkyl, or heterocycloalkylalkyl or R³³ and R³⁴ together with Si form a heterocycloalkylene ring containing the Si atom and 3 to 7 carbon ring atoms wherein one or two carbon ring atoms are optionally independently replaced with —NH—, —O—, —S—, —SO—, —SO₂—, —CO—, —CONH—, or —SO₂NH— and wherein the aralkyl, heteroaralkyl, heterocycloalkyl, or heterocycloalkylene ring in R^{1a} is optionally substituted on the ring with one, two, or three R^{e} independently selected from alkyl, haloalkyl, alkoxy, hydroxy, haloalkoxy, halo, carboxy, alkoxycarbonyl, amino, monsubstituted amino, disubstituted amino, nitro, aryloxy, benzyloxy, acyl, alkylsulfonyl, or arylsulfonyl and further wherein the aromatic or alicyclic ring in R^{e} is optionally substituted with one or two substituents independently selected from alkyl, halo, alkoxy, haloalkyl, haloalkoxy, hydroxy, amino, alkylamino, dialkylamino, carboxy, or alkoxycarbonyl;

R² is hydrogen or alkyl;

R³ is alkyl, haloalkyl, cycloalkyl, cycloalkylalkyl, aryl, aralkyl, heteroaryl, heteroaralkyl, heterocycloalkyl, heterocycloalkylalkyl, or -alkylene-X⁶—R³⁵ [wherein X⁶ is —NR³⁶—, —O—, —S(O)ₙ₄—, —CO—, —COO—, —OCO—, —NR³⁶CO—, —CONR³⁶—, —NR³⁶SO₂—, —SO₂NR³⁶—, —NR³⁶COO—, —OCONR³⁶—, —NR³⁶CONR³⁷—, or —NR³⁶SO₂NR³⁷— (where each R³⁶ and R³⁷ is independently hydrogen, alkyl, or acyl and n4 is 0-2) and R³⁵ is hydrogen, alkyl, haloalkyl, cycloalkyl, cycloalkylalkyl, heterocycloalkyl, heterocycloalkylalkyl, aryl, aralkyl, heteroaryl, or heteroaralkyl] wherein the alkylene chain in R³ is optionally substituted with one to four halo atoms and the aromatic and alicyclic rings in R³ are optionally substituted by one, two, or three R^{f} independently selected from alkyl, aminoalkyl, halo, hydroxy, alkoxy, haloalkyl, haloalkoxy, oxo, cyano, nitro, acyl, acyloxy, aryl, aralkyl, heteroaryl, heteroaralkyl, cycloalkyl, cycloalkylalkyl, heterocycloalkyl, heterocycloalkylalkyl, aryloxy, benzyloxy, carboxy, alkoxycarbonyl, aryloxycarbonyl, carbamoyl, alkylthio, alkylsulfinyl, alkylsulfonyl, arylthio, arylsulfonyl, arylsulfinyl, alkoxycarbonylamino, aryloxycarbonylamino, alkylcarbamoyloxy, arylcarbamoyloxy, alkylsulfonylamino, arylsulfonylamino, aminosulfonyl, alkylaminosulfonyl, dialkylaminosulfonyl, arylaminosulfonyl, aralkylaminosulfonyl, aminocarbonyl, arylaminocarbonyl, aralkylaminocarbonyl, amino, monosubsituted or disubstituted amino, and further wherein the aromatic and alicyclic rings in R^{f} are optionally substituted with one, two, or three R^{g} wherein R^{g} is independently selected from alkyl, halo, haloalkyl, haloalkoxy, hydroxy, nitro, cyano, hydroxyalkyl, alkoxy, alkoxyalkyl, aminoalkyl, alkylthio, alkylsulfonyl, amino, monosubstituted amino, dialkylamino, aryl, heteroaryl, cycloalkyl, carboxy, carboxamido, or alkoxycarbonyl; or

a pharmaceutically acceptable salts thereof.

Preferably, the disease is juvenile onset diabetes, psoriasis, multiple sclerosis, pemphigus vulgaris, Graves' disease, myasthenia gravis, systemic lupus erythemotasus, rheumatoid arthritis, Hashimoto's thyroiditis, allergic disorders including, but not limited to, asthma, allogeneic immune responses including, but not limited to, organ transplants or tissue grafts and endometriosis, chronic obstructive pulmonary disease (e.g., emphysema), bronchiolitis, excessive airway elastolysis in asthma and bronchitis, pneumonities and cardiovascular disease such as plaque rupture and atheroma, systemic amyloidosis, Alzheimer's disease, and iatrogenic disorders. Preferably, the disease is psoriasis, iratrogenic disorders, and myasthenia gravis.

In a third aspect this invention is directed to a pharmaceutical composition comprising a compound of Formula (I) or a pharmaceutically acceptable salt thereof, in admixture with a suitable excipient.

In a fourth aspect this invention is directed to a method of treating a patient undergoing a therapy wherein the therapy causes an immune response in the patient comprising administering to the patient a compound of Formula (I) or a pharmaceutically acceptable salt thereof. Preferably, the immune response is mediated by MHC class II molecules. The compound of Formula (I) can be administered prior to, simultaneously, or after the therapy. Preferably, the therapy involves treatment with a biologic. Preferably, the therapy involves treatment with a small molecule.

Preferably, the biologic is a protein, preferably an antibody, more preferably a monoclonal antibody. More preferrably, the biologic is Remicade®, Refacto®, Referon-A®, Factor VIII, Factor VII, Betaseron®, Epogen®, Embrel®, Interferon beta, Botox®, Fabrazyme®, Elspar®, Cerezyme®, Myobloc®, Aldurazyme®, Verluma®, Interferon alpha, Humira®, Aranesp®, Zevalin® or OKT3.

Preferably, the small molecule therapy involves use of heparin, low molecular weight heparin, procainamide or hydralazine.

In a fifth aspect, this invention is directed to a method of treating immune response in an animal that is caused by administration of a biologic to the animal which method comprises administering to the animal in need of such treatment a therapeutically effective amount of a compound of Formula (I) or a pharmaceutically acceptable salt thereof.

In a sixth aspect, this invention is directed to a method of conducting a clinical trial for a biologic comprising administering to an individual participating in the clinical trial a compound of Formula (I) or a pharmaceutically acceptable salt thereof with the biologic.

In a seventh aspect, this invention is directed to a method of prophylactically treating a person undergoing treatment with a biologic with a compound of Formula (I) or a pharmaceutically acceptable salt thereof to treat the immune response caused by the biologic in the person.

In an eighth aspect, this invention is directed to a method of determining the loss in the efficacy of a biologic in an animal due to the immune response caused by the biologic comprising administering the biologic to the animal in the presence and absence of a compound of Formula (I) or a pharmaceutically acceptable salt thereof.

In a ninth aspect, this invention is directed to a method of improving efficacy of a biologic in an animal comprising administering the biologic to the animal with a compound of Formula (I) or a pharmaceutically acceptable salt thereof.

In a tenth aspect, this invention is directed to the use of a compound of Formula (I) or a pharmaceutically acceptable salt thereof for the manufacture of a medicament.

In a eleventh aspect, this invention is directed to the use of a compound of Formula (I) or a pharmaceutically acceptable salt thereof for the manufacture of a medicament for combination therapy with a biologic, to treat the immune response caused by the biologic.

Preferably, the Cathepsin S inhibitor is administered prior to the administration of the biological agent.

Preferably, the Cathepsin S inhibitor is administered concomitantly with the biological agent.

Preferably, the Cathepsin S inhibitor is administered after the administration of the biological agent.

### DETAILED DESCRIPTION OF THE INVENTION

Definitions:

Unless otherwise stated, the following terms used in the specification and claims are defined for the purposes of this application and have the following meanings.

“Alicyclic” means cycloalkyl and heterocycloalkyl rings as defined herein.

“Alkyl” represented by itself means a straight or branched, saturated aliphatic radical containing one to six carbon atoms, unless otherwise indicated e.g., alkyl includes methyl, ethyl, propyl, isopropyl, butyl, sec-butyl, isobutyl, tert-butyl, and the like.

“Alkenyl” represented by itself means a straight or branched, aliphatic radical of two to six carbon atoms containing one or two double bond e.g., ethenyl, propenyl, and the like.

“Alkylene”, unless indicated otherwise, means a straight or branched, saturated aliphatic, divalent radical having one to six carbon atoms, e.g., methylene (—CH₂—), ethylene (—CH₂CH₂—), trimethylene (—CH₂CH₂CH₂—), tetramethylene (—CH₂CH₂CH₂CH₂—) 2-methyltetramethylene (—CH₂CH(CH₃)CH₂CH₂—), pentamethylene (—CH₂CH₂CH₂CH₂CH₂—), and the like.

“Alkylcarbamoyloxy” refers to a —OCONHR radical where R is an alkyl group as defined above e.g., methylcarbamoyloxy, ethylcarbamoyloxy, and the like.

“Alkylsulfonylamino” refers to a —NHSO₂R radical where R is an alkyl group as defined above e.g., methylsulfonylamino, ethylsulfonylamino, and the like.

“Amino” means the —NH₂ radical.

“Aminosulfonyl” refers to the —SO₂NH₂ radical.

“Alkylaminosulfonyl” or “dialkylaminosulfonyl” refers to a —SO₂NHR and —SO₂NRR′ radical respectively, where R and R′ are independently alkyl group as defined above e.g., methylaminosulfonyl, dimethylaminosulfonyl, and the like.

“Alkylamino” or “dialkylamino” refers to a —NHR and —NRR′ radical respectively, where R and R′ are independently alkyl group as defined above e.g., methylamino, dimethylamino, and the like.

“Alkoxy” refers to a —OR radical where R is an alkyl group as defined above e.g., methoxy, ethoxy, and the like.

“Alkoxycarbonyl” refers to a —C(O)OR radical where R is an alkyl group as defined above e.g., methoxycarbonyl, ethoxycarbonyl, and the like.

“Alkoxycarbonylalkyl” means a -(alkylene)-C(O)OR radical where R is alkyl as defined above e.g., methoxycarbonylalkyl, 2-, or 3-ethoxycarbonylpropyl, and the like.

“Alkoxycarbonylamino” refers to a —NHC(O)OR radical where R is an alkyl group as defined above e.g., methoxycarbonylamino, ethoxycarbonylamino, and the like.

“Alkoxyalkyl” means a linear monovalent hydrocarbon radical of one to six carbon atoms or a branched monovalent hydrocarbon radical of three to six carbons substituted with at least one alkoxy group, preferably one or two alkoxy groups, as defined above, e.g., 2-methoxyethyl, 1-, 2-, or 3-methoxypropyl, 2-ethoxyethyl, and the like.

“Alkoxyalkyloxyalkyl” refers to a -(alkylene)-O-(alkylene)-OR radical where R is an alkyl group as defined above, e.g., 2-methoxyethyloxymethyl, 3-methoxypropyloxyethyl, and the like.

“Aminoalkyl” means a linear monovalent hydrocarbon radical of one to six carbon atoms or a branched monovalent hydrocarbon radical of three to six carbons substituted with at least one, preferably one or two, —NRR′ where R is hydrogen, alkyl, or —COR^{a} where R^{a} is alkyl, and R′ is hydrogen or alkyl as defined above e.g., aminomethyl, methylaminoethyl, dimethylaminoethyl, 1,3-diaminopropyl, acetylaminopropyl, and the like.

“Alkylthio” refers to a —SR radical where R is an alkyl group as defined above e.g., methylthio, ethylthio, and the like.

“Alkylsulfinyl” refers to a —S(O)R radical where R is an alkyl group as defined above e.g., methylsylfinyl, ethylsulfinyl, and the like.

“Alkylsulfonyl” refers to a —SO₂R radical where R is an alkyl group as defined above e.g., methylsulfonyl, ethylsulfonyl, and the like.

“Acyl” means a —COR radical where R is hydrogen, alkyl, haloalkyl, aryl, aralkyl, heteroaryl, heteroaralkyl, or heterocycloalkyl as defined herein, e.g., formyl, acetyl, trifluoroacetyl, benzoyl, piperazin-1-ylcarbonyl, and the like.

“Acyloxy” means a —OCOR radical where R is alkyl, haloalkyl, aryl, aralkyl, heteroaryl, heteroaralkyl, or heterocycloalkyl as defined herein, e.g., acetyloxy, trifluoroacetyloxy, benzoyloxy, piperazin-1-ylcarbonyloxy, and the like.

“Animal” includes humans, non-human mammals (e.g., dogs, cats, rabbits, cattle, horses, sheep, goats, swine, deer, and the like) and non-mammals (e.g., birds, and the like).

“Aromatic” means a moiety wherein the constituent atoms make up an unsaturated ring system, all atoms in the ring system are sp² hybridized and the total number of pi electrons is equal to 4n+2.

“Aryl” means a monocyclic or fused bicyclic ring assembly containing 6 to 10 ring carbon atoms unless otherwise indicated, wherein each ring is aromatic e.g., phenyl or naphthyl.

“Aralkyl” means a -(alkylene)-R radical where R is aryl as defined above e.g., benzyl, phenethyl, and the like.

“Aryloxy” means a —OR radical where R is aryl as defined above.

“Aryloxyalkyl” means a -(alkylene)-OR radical where R is aryl as defined above e.g., phenoxymethyl, 2-, or 3-phenoxypropyl, and the like

“Aryloxycarbonyl” means a —C(O)OR radical where R is aryl as defined above e.g., phenyloxycarbonyl, and the like.

“Arylcarbamoyloxy” means a —OC(O)NHR radical where R is aryl as defined above e.g., phenylcarbamoyloxy, and the like.

“Arylthio” refers to a —SR radical where R is an aryl group as defined above e.g., phenylthio, and the like.

“Arylsulfinyl” refers to a —SOR radical where R is an aryl group as defined above e.g., phenylsulfinyl, and the like.

“Arylsulfonyl” refers to a —SO₂R radical where R is an aryl group as defined above e.g., phenylsulfonyl, and the like.

“Aryloxycarbonylamino” refers to a —NHC(O)OR radical where R is an aryl group as defined above e.g., phenoxycarbonylamino, and the like.

“Arylsulfonylamino” refers to a —NHSO₂R radical where R is an aryl group as defined above, e.g., phenylsulfonylamino, and the like.

“Arylaminosulfonyl” means a —SO₂NHR radical where R is aryl as defined above e.g., phenylaminosulfonyl, and the like.

“Aralkylaminosulfonyl” means a —SO₂NHR radical where R is aralkyl as defined above e.g., benzylaminosulfonyl, and the like.

“Arylaminocarbonyl” means a —CONHR radical where R is aryl as defined above e.g., phenylaminocarbonyl, and the like.

“Aralkylaminocarbonyl” means a —CONHR radical where R is aralkyl as defined above e.g., benzylaminocarbonyl, and the like.

“Biologic” means a therapeutic agent originally derived from living organisms for the treatment or management of a disease. Examples include, but are not limited to, proteins (recombinant and plasma derived), e.g., monoclonal or polyclonal, humanized or murine antibodies, toxins, hormones, and the like. Biologics are currently available for the treatment of a variety of diseases such as cancer, rheumatoid arthritis, and haemophilia.

“Carbamoyl” or “aminocarbonyl” means a —C(O)NRR′ radical where R and R′ are independently selected from hydrogen, alkyl, aryl, aralkyl, heteroaryl, heteroaralkyl, heterocycloalkyl or heterocycloalkylalkyl as provided herein provided one of R and R′ is not hydrogen.

“Carboxy” means the radical —C(O)OH.

“Cycloalkyl” means a monovalent saturated or partially unsaturated, monocyclic, fused bicyclic ring assembly containing three to eight ring carbon atoms e.g., cyclopropyl, cyclobutyl, cyclopentyl, cyclohexyl, cyclohexenyl, and the like.

“Cycloalkylalkyl” means a -(alkylene)-R radical where R is cycloalkyl as defined above e.g., cyclopropylmethyl, cyclobutylethyl, cyclobutylmethyl, and the like

“Cycloalkylene” means a divalent saturated or partially unsaturated monocyclic ring or fused ring assembly containing three to eight ring carbon atoms. For example, the instance wherein “R⁵ and R⁶ together with the carbon atom to which both R⁵ and R⁶ are attached form cycloalkylene” includes, but is not limited to, the following:

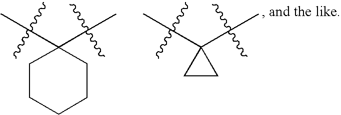

“Disubstituted amino” means a —NRR′ radical where R is alkyl, aryl, aralkyl, heteroaryl, heteraralkyl, or heterocycloalkyl, and R′ is alkyl, aryl, aralkyl, heteroaryl, heteroaralkyl, cycloalkyl, heterocycloalkyl, cycloalkylalkyl, hydroxyalkyl, alkoxyalkyl, or acyl as defined herein. Representative examples include, but are not limited to, dimethylamino, methylphenylamino, benzylmethylamino, acetylmethylamino, and the like.

“1,1-Dialkylsilinan-4-ylalkylene” means a group having the structure depicted below: 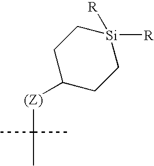
where Z is alkylene and each R is independently alkyl as defined herein.

“Derived” means a similar agent can be traced to.

“Disease” specifically includes any unhealthy condition of an animal or part thereof and includes an unhealthy condition that may be caused by, or incident to, medical or veterinary therapy applied to that animal, i.e., the “side effects” of such therapy.

“Deleterious immune response” means an immune response that prevents effective treatment of a patient or causes disease in a patient. As an example, dosing a patient with a murine antibody either as a therapy or a diagnostic agent causes the production of human antimouse antibodies that prevent or interfere with subsequent treatments, The incidence of antibody formation versus pure murine monoclonals can exceed 70%. (see Khazaeli, M. B. et al. J. Immunother. 1994, 15, pp 42-52; Dillman R. O. et al. Cancer Biother. 1994, 9, pp 17-28; and Reinsberg, J. Hybridoma. 1995, 14, pp 205-208). Additional examples of known agents that suffer from deleterious immune responses are blood-clotting factors such as factor VIII. When administered to hemophilia A patients, factor VIII restores the ability of the blood to clot. Although factor VIII is a human protein, it still elicits an immune response in hemophiliacs as endogenous factor VIII is not present in their blood and thus it appears as a foreign antigen to the immune system. Approximately 29-33% of new patients will produce antibodies that bind and neutralize the therapeutically administered factor VIII (see Lusher J. M. Semin Thromb Hemost. 2002, 28(3), pp 273-276). These neutralizing antibodies require the administration of larger amounts of factor VIII in order to maintain normal blood clotting parameters; an expensive regimen of treatment in order to induce immune tolerance (see Briet E et al. Adv. Exp. Med. Bio. 2001, 489, pp 89-97). Another immunogenic example is adenoviral vectors. Retroviral therapy remains experimental and is of limited utility. One reason is that the application of a therapeutic virus generates an immune response capable of blocking any subsequent administration of the same or similar virus (see Yiping Yang et al. J. of Virology. 1995, 69, pp 2004-2015). This ensures that retroviral therapies must be based on the transient expression of a protein or the direct incorporation of viral sequence into the host genome. Directed research has identified multiple viral neutralizing epitopes recognized by host antibodies (see Hanne, Gahery-Segard et al. J. of Virology 1998. 72, pp 2388-2397) suggesting that viral modifications will not be sufficient to overcome this obstacle. This invention will enable a process whereby an adenoviral therapy will have utility for repeated application. Another example of an immunogenic agent that elicits neutralizing antibodies is the well-known cosmetic agent Botox. Botulin toxin protein, is purified from the fermentation of Clostridium botulinum. As a therapeutic agent, it is used for muscle disorders such as cervical dystonia in addition to cosmetic application. After repeated exposure patients generate neutralizing antibodies to the toxin that results in reduced efficacy (see Birklein F. et al. Ann Neurol. 2002, 52, pp 68-73 and Rollnik, J. D. et al. Neurol. Clin. Neurophysiol. 2001, 2001(3), pp 2-4). A “deleterious immune response” also encompasses diseases caused by therapeutic agents. A specific example of this is the immune response to therapy with recombinant human erythropoietin (EPO). Erythropoietin is used to stimulate the growth or red cells and restore red blood cell counts in patients who have undergone chemotherapy or dialysis. A small percentage of patients develop antibodies to EPO and subsequently are unresponsive to both therapeutically administered EPO and their own endogenous EPO (see Casadevall, N. et al., NEJM. 2002, 346, pp 469-475). They contract a disorder, pure red cell aplasia, in which red blood cell production is severely diminished (see Gershon S. K. et. al. NEJM. 2002, 346, pp 1584-1586). This complication of EPO therapy is lethal if untreated. Another specific example is the murine antibody, OKT3 (a.k.a., Orthoclone) a monoclonal antibody directed towards CD-3 domain of activated T-cells. In clinical trials 20-40% of patients administered OKT3 produce antibodies versus the therapy. These antibodies, besides neutralizing the therapy, also stimulate a strong host immune reaction. The immune reaction is severe enough that patients with high titers of human anti-mouse antibodies are specifically restricted from taking the drug (see Orthoclone package label). A final example is a human antibody therapeutic. Humira® is a monoclonal antibody directed against TNF and is used to treat rheumatoid arthritis patients. When taken alone ˜12% of patients develop neutralizing antibodies. In addition, a small percentage of patients given the drug also contract a systemic lupus erthematosus-like condition that is an IgG-mediated immune response induced by the therapeutic agent (see Humira package label).

Another example of “deleterious immune response” is a host reaction to small molecule drugs. It is known to those skilled in the art that certain chemical structures will conjugate with host proteins to stimulate immune recognition (see Ju. C. et al. 2002. Current Drug Metabolism 3, pp 367-377 and Kimber I. et al. 2002, Toxicologic Pathology 30, pp 54-58.) A substantial portion of these host reactions are IgG mediated. Specific “deleterious immune responses” that are IgG mediated include: hemolytic anemia, Steven-Johnson syndrome and drug induced Lupus.

“Halo” means fluoro, chloro, bromo or iodo.

“Haloalkyl” means alkyl substituted by one or more, preferably one to five, “halo” atoms, as such terms are defined in this application. Haloalkyl includes monohaloalkyl, dihaloalkyl, trihaloalkyl, perhaloalkyl and the like e.g. chloromethyl, dichloromethyl, difluoromethyl, trifluoromethyl, 2,2,2-trifluoroethyl, perfluoroethyl, 2,2,2-trifluoro-1,1-dichloroethyl, and the like).

“Haloalkoxy” refers to a —OR radical where R is haloalkyl group as defined above e.g., trifluoromethoxy, 2,2,2-trifluoroethoxy, difluoromethoxy, and the like.

“Heteroaryl” means an aromatic monocyclic or multicyclic ring of 5 to 10 ring atoms in which one or more, preferably one, two, or three, of the ring atoms are selected from nitrogen, oxygen or sulfur, the remaining ring atoms being carbon. Representative heteroaryl rings include, but are not limited to, pyrrolyl, furanyl, thienyl, oxazolyl, isoxazolyl, thiazolyl, imidazolyl, triazolyl, tetrazolyl, pyridinyl, pyrimidinyl, pyrazinyl, pyridazinyl, indolyl, benzofuranyl, benzothienyl, benzimidazolyl, quinolinyl, isoquinolinyl, quinazolinyl, quinoxalinyl, pyrazolyl, and the like.

“Heteroaralkyl” means a -(alkylene)-R radical where R is heteroaryl as defined above e.g., pyridinylmethyl, 1- or 2-furanylethyl, imidazolylmethyl, and the like.

“Heteroaryloxyalkyl” means a -(alkylene)-OR radical where R is heteroaryl as defined above e.g., furanyloxymethyl, 2-, or 3-indolyloxyethyl, and the like.

“Heteroarylsulfonyl” refers to a —SO₂R radical where R is an heteroaryl group e.g., pyridinylsulfonyl, and the like.

“Heterocycloalkyl” means cycloalkyl, as defined in this application, provided that one or more, preferably one, two, or three of the ring carbon atom(s) indicated are replaced by a heteroatom selected from —N—, —O—, —S—, —SO—, or —S(O)₂— and additionally where one or two carbon atoms are optionally replaced by —C(O)—. Representative examples include, but are not limited to, imidazolidinyl, morpholinyl, thiomorpholinyl, thiomorpholino-1-oxide, thiomorpholino-1,1-dioxide, tetrahydropyranyl, tetrahydrothiopyranyl, 1-oxo-tetrahydrothiopyranyl, 1,1-dioxotetrathiopyranyl, indolinyl, piperazinyl, piperidyl, pyrrolidinyl, pyrrolinyl, quinuclidinyl, and the like.

“Heterocycloalkylalkyl” means a -(alkylene)-heterocycloalkyl radical where heterocycloalkyl is as defined in this application. Representative examples include, but are not limited to, imidazolidin-1-ylmethyl, morpholin-4-ylmethyl, thiomorpholin-4-ylmethyl, thiomorpholin-4-ylmethyl-1-oxide, indolinylethyl, piperazinylmethyl or -ethyl, piperidylmethyl or -ethyl, pyrrolidinylmethyl or -ethyl, and the like.

“Heterocycloalkylene” means cycloalkylene, as defined in this application, provided that one or more, preferably one or two, of the ring member carbon atoms is replaced by a heteroatom selected from —N—, —O—, —S— or —S(O)₂— and optionally one or two ring member carbon atom(s) are replaced with —C(O)—. For example, the instance wherein R⁵ and R⁶ together with the carbon atom to which both R⁵ and R⁶ are attached form heterocycloalkylene” includes, but is not limited to, the following: 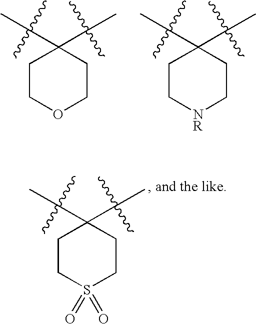
in which R is a substituent defined in the Summary of the Invention.

“Hydroxy” means the —OH radical.

“Hydroxyalkyl” means a linear monovalent hydrocarbon radical of one to six carbon atoms or a branched monovalent hydrocarbon radical of three to six carbons substituted with one or two hydroxy groups, provided that if two hydroxy groups are present they are not both on the same carbon atom. Representative examples include, but are not limited to, hydroxymethyl, 2-hydroxyethyl, 2-hydroxypropyl, 3-hydroxypropyl, 1-(hydroxymethyl)-2-methylpropyl, 2-hydroxybutyl, 3-hydroxybutyl, 4-hydroxybutyl, 2,3-dihydroxypropyl, 1-(hydroxymethyl)-2-hydroxyethyl, 2,3-dihydroxybutyl, 3,4-dihydroxybutyl and 2-(hydroxymethyl)-3-hydroxypropyl, preferably 2-hydroxyethyl, 2,3-dihydroxypropyl, and 1-(hydroxymethyl)-2-hydroxyethyl.

“Isomers” mean compounds of the present invention having identical molecular formulae but differ in the nature or sequence of bonding of their atoms or in the arrangement of their atoms in space. Isomers that differ in the arrangement of their atoms in space are termed “stereoisomers”. Stereoisomers that are not mirror images of one another are termed “diastereomers” and stereoisomers that are nonsuperimposable mirror images are termed “enantiomers” or sometimes “optical isomers”. A carbon atom bonded to four nonidentical substituents is termed a “chiral center”. A compound with one chiral center has two enantiomeric forms of opposite chirality is termed a “racemic mixture”. A compound that has more than one chiral center has 2ⁿ⁻¹ enantiomeric pairs, where n is the number of chiral centers. Compounds with more than one chiral center may exist as ether an individual diastereomers or as a mixture of diastereomers, termed a “diastereomeric mixture”. When one chiral center is present a stereoisomer may be characterized by the absolute configuration of that chiral center. Absolute configuration refers to the arrangement in space of the substituents attached to the chiral center. Enantiomers are characterized by the absolute configuration of their chiral centers and described by the R- and S-sequencing rules of Cahn, Ingold and Prelog.

Conventions for stereochemical nomenclature, methods for the determination of stereochemistry and the separation of stereoisomers are well known in the art (e.g., see “Advanced Organic Chemistry”, 4th edition, March, Jerry, John Wiley & Sons, New York, 1992). It is understood that the names and illustration used in this application to describe compounds of Formula (I) are meant to be encompassed all possible stereoisomers.

Additionally, compounds of Formula (I) may exist as tautomers. Such tautomeric forms (individual tautomers or mixtures thereof) are within the scope of this invention.

“Keto or oxo” means the radical (═O).

“Monosubstituted amino” means a —NHR radical where R is alkyl, aryl, aralkyl, heteroaryl, heteroaralkyl, cycloalkyl, cycloalkylalkyl, hydroxyalkyl, alkoxyalkyl, or acyl as defined herein. Representative examples include, but are not limited to, methylamino, phenylamino, benzylamino, cyclopropylmethylamino, acetylamino, trifluoroacetyl, and the like.

“Nitro” means the —NO₂ radical.

“Optional” or “optionally” or “may be” means that the subsequently described event or circumstance may or may not occur, and that the description includes instances where the event or circumstance occurs and instances in which it does not. For example, the phrase “wherein the aromatic ring R^{a} is optionally substituted with one or two substituents independently selected from alkyl, . . . ” means that the aromatic ring in R^{a} may or may not be substituted with alkyl in order to fall within the scope of the invention. Additionally, the phase “wherein R³³ and R³⁴ together with Si form a heterocycloalkylene ring containing the Si atom and 3 to 7 carbon ring atoms wherein one or two carbon ring atoms are optionally independently replaced with —NH—, —O—, —S—, —SO—, —SO₂—, —CO—, —CONH—, or —SO₂NH— and wherein the heterocycloalkylene ring in R^{1a} is optionally substituted on the ring with one, two, or three R^{e} independently selected from alkyl, . . . ” means the hydrogen the —NH— group in the heterocycloalkylene ring may or may not be substituted with alkyl in order to fall within the scope of the invention.

The present invention also includes N-oxide derivatives of the compounds of this invention. N-oxide derivatives means derivatives of compounds of the present invention in which nitrogens are in an oxidized state (i.e., N→O) e.g., pyridine N-oxide, and which possess the desired pharmacological activity.

“Pathology” of a disease means the essential nature, causes and development of the disease as well as the structural and functional changes that result from the disease processes.

“Pharmaceutically acceptable” means that which is useful in preparing a pharmaceutical composition and is generally safe, non-toxic and neither biologically nor otherwise undesirable and includes that which is acceptable for veterinary use as well as human pharmaceutical use.

“Pharmaceutically acceptable salts” means salts of compounds of the present invention which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as acetic acid, propionic acid, hexanoic acid, heptanoic acid, cyclopentanepropionic acid, glycolic acid, pyruvic acid, lactic acid, malonic acid, succinic acid, malic acid, maleic acid, fumaric acid, tartaric acid, citric acid, benzoic acid, o-(4-hydroxybenzoyl)benzoic acid, cinnamic acid, mandelic acid, methylsulfonic acid, ethanesulfonic acid, 1,2-ethanedisulfonic acid, 2-hydroxyethanesulfonic acid, benzenesulfonic acid, p-chlorobenzenesulfonic acid, 2-naphthalenesulfonic acid, p-toluenesulfonic acid, camphorsulfonic acid, 4-methylbicyclo[2.2.2]oct-2-ene-1-carboxylic acid, glucoheptonic acid, 4,4′-methylenebis(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid and the like.

Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like.

The present invention also includes prodrugs of a compound of the present invention. Prodrug means a compound that is convertible in vivo by metabolic means (e.g. by hydrolysis) to a compound of the present invention. For example an ester of a compound of the present invention containing a hydroxy group may be convertible by hydrolysis in vivo to the parent molecule. Alternatively an ester of a compound of the present invention containing a carboxy group may be convertible by hydrolysis in vivo to the parent molecule. Suitable esters of compounds of the present invention containing a hydroxy group, are for example acetates, citrates, lactates, tartrates, malonates, oxalates, salicylates, propionates, succinates, fumarates, maleates, methylene-bis-b-hydroxynaphthoates, gentisates, isethionates, di-p-toluoyltartrates, methylsulphonates, ethanesulphonates, benzenesulphonates, p-toluenesulphonates, cyclohexylsulphamates and quinates. Suitable esters of compounds of the present invention containing a carboxy group, are for example those described by Leinweber, F. J. Drug Metab. Res., 1987, 18, pg. 379. An especially useful class of esters of compounds of the present invention containing a hydroxy group, may be formed from acid moieties selected from those described by Bundgaard et al., J. Med. Chem., 1989, 32, page 2503-2507, and include substituted (aminomethyl)-benzoates, for example, dialkylamino-methylbenzoates in which the two alkyl groups may be joined together and/or interrupted by an oxygen atom or by an optionally substituted nitrogen atom, e.g. an alkylated nitrogen atom, more especially (morpholino-methyl)benzoates, e.g. 3- or 4-(morpholinomethyl)-benzoates, and (4-alkylpiperazin-1-yl)benzoates, e.g. 3- or 4-(4-alkylpiperazin-1-yl)benzoates.

“Protected derivatives” means derivatives of compounds of the present invention in which a reactive site or sites are blocked with protecting groups. Protected derivatives of compounds of the present invention are useful in the preparation of compounds of the present invention or in themselves may be active cathepsin S inhibitors. A comprehensive list of suitable protecting groups can be found in T. W. Greene, Protecting Groups in Organic Synthesis, 3rd edition, John Wiley & Sons, Inc. 1999.

“The expression wherein the aromatic or alicyclic ring in R⁶, R^{6a}, R^{a}, R¹⁰, R²³ . . . etc., is optionally substituted with alkyl, haloalkyl . . . ” includes both aromatic or alicylic ring that is directly attached or is part of a group that is attached to the specified group e.g., R⁶, R^{6a}, . . . etc. For example, the expression R²³ is selected from hydrogen, alkyl, haloalkyl, hydroxyalkyl, alkoxyalkyl, aryloxyalkyl, heteroaryloxyalkyl, aminoalkyl, acyl, aryl, aralkyl, heteroaryl, heteroaralkyl, cycloalkyl, cycloalkylalkyl, —S(O)₂R²⁴, -alkylene-S(O)ₙ₃—R²⁵, —COOR²⁶, -alkylene-COOR²⁷, —CONR²⁸R²⁹, or -alkylene-CONR³⁰R³¹ (where n3 is 0-2 and R²⁴-R²⁷, R²⁸ and R³⁰ are independently hydrogen, alkyl, haloalkyl, aryl, aralkyl, heteroaryl, heteroaralkyl, cycloalkyl, cycloalkylalkyl, heterocycloalkyl, or heterocycloalkylalkyl and R²⁹ and R³¹ are independently hydrogen or alkyl) where the aromatic or alicyclic ring in R²³ is optionally substituted with one, two, or three substituents independently selected from alkyl, haloalkyl, alkoxy, haloalkoxy, halo, hydroxy, amino, alkylamino, dialkylamino, carboxy, or alkoxycarbonyl and one substitutent selected from aryl, aralkyl, heteroaryl, or heteroaralkyl includes aromatic and alicyclic rings such as aryl, aralkyl, cycloalkylalkyl, and aromatic or alicylic ring in -alkylene-S(O)ₙ₃—R²⁵ group where R²⁵ is aryl, aralkyl, cycloalkyl, . . . etc.

“Therapeutically effective amount” means that amount which, when administered to an animal for treating a disease, is sufficient to effect such treatment for the disease.

“Treatment” or “treating” means any administration of a compound of the present invention and includes:

(1) preventing the disease from occurring in an animal which may be predisposed to the disease but does not yet experience or display the pathology or symptomatology of the disease,

(2) inhibiting the disease in an animal that is experiencing or displaying the pathology or symptomatology of the diseased (i.e., arresting further development of the pathology and/or symptomatology), or

(3) ameliorating the disease in an animal that is experiencing or displaying the pathology or symptomatology of the diseased (i.e., reversing the pathology and/or symptomatology).

“Treatment” or “treating” with respect to combination therapy i.e., use with a biologic means any administration of a compound of the present invention and includes:

(1) preventing the immune response from occurring in an animal which may be predisposed to the immune response but does not yet experience or display the pathology or symptomatology of the immune response,

(2) inhibiting the immune response in an animal that is experiencing or displaying the pathology or symptomatology of the immune response (i.e., arresting further development of the pathology and/or symptomatology), or

(3) ameliorating the immune response in an animal that is experiencing or displaying the pathology or symptomatology of the immune response (i.e., reducing in degree or severity, or extent or duration, the overt manifestations of the immune response or reversing the pathology and/or symptomatology e.g., reduced binding and presentation of antigenic peptides by MHC class II molecules, reduced activation of T-cells and B-cells, reduced humoral and cell-mediated responses and, as appropriate to the particular immune response, reduced inflammation, congestion, pain, necrosis, reduced loss in the efficacy of a biologic agent, and the like).

### Preferred Embodiments

While the broadest definition of this invention is set forth in the Summary of the Invention, certain compounds of this invention are preferred. For example:

A. One preferred group of compounds is that wherein E is —C(R⁵)(R⁶)X¹ in which:

R⁵ is hydrogen or alkyl; and

R⁶ is hydrogen, alkyl, -(alkylene)-OR¹² (where R¹² is hydrogen, alkyl or haloalkyl), cycloalkyl, cycloalkylalkyl, aryl, aralkyl, heteroaryl, heteroaralkyl, heterocycloalkyl, or heterocycloalkylalkyl wherein aryl, aralkyl, heteroaryl, heteroaralkyl, heterocycloalkyl or heterocycloalkylalkyl is optionally substituted with one, two, or three R^{a} independently selected from alkyl, haloalkyl, alkoxy, hydroxy, haloalkoxy, halo, carboxy, alkoxycarbonyl, amino, monsubstituted amino, disubstituted amino, or acyl.

Preferably, R⁵ is hydrogen;

R⁶ is alkyl, preferably ethyl or propyl, more preferably ethyl; and

X¹ is —CHO, —C(O)R¹⁰, —C(O)CF₃, —C(O)CF₂CF₂R⁹—CH═CHS(O)₂R¹⁰, —C(O)CF₂C(O)NR¹⁰R¹¹, —C(O)C(O)NR¹⁰R¹¹, —C(O)CH₂OR¹⁰, —C(O)CH₂N(R¹¹)SO₂R¹⁰, —C(O)C(O)N(R¹¹)(CH₂)₂OR¹¹, —C(O)C(O)N(R¹¹)(CH₂)₂NHR¹¹ or —C(O)C(O)R¹⁰ wherein R¹⁰ is alkyl, aryl, aralkyl, heteroaryl, heteroaralkyl, cycloalkylalkyl or heterocycloalkylalkyl wherein the aromatic ring in R¹⁰ is optionally substituted with R^{d} selected from heteroaryl, aryl, alkyl, or alkoxyalkyl R¹¹ is hydrogen or alkyl and R⁹ is halo. More preferably, X¹ is —C(O)C(O)NHR¹¹ where R¹¹ is cycloalkyl, preferably cyclopropyl.

More preferably, E is —CHR⁶C(O)R¹⁰ where R⁶ is alkyl, preferably ethyl, propyl, or butyl, more preferably ethyl, and R¹⁰ is heteroaryl optionally substituted with one or two R^{d} independently selected from alkyl, haloalkyl, alkoxy, alkoxyalkyl, cycloalkyl, hydroxy, haloalkoxy, halo, carboxy, alkoxycarbonyl, aryl, heteroaryl, amino, monsubstituted amino, disubstituted amino, or acyl wherein the aromatic or alicyclic ring in R^{d} is optionally substituted with one, two, or three substitutents independently selected from alkyl, haloalkyl, alkoxy, haloalkoxy, halo, hydroxy, carboxy, alkoxycarbonyl, amino, alkylamino, or dialkylamino. More preferably, R¹⁰ is benzoxazol-2-yl, 4-azabenzoxazol-2-yl, 2-pyridin-3-yl-[1,3,4]-oxadiazol-5-yl, 2-pyridin-4-yl-[1,3,4]-oxadiazol-5-yl, 2-ethyl-[1,3,4]-oxadiazol-5-yl, 2-isopropyl-[1,3,4]-oxadiazol-5-yl, 2-tert-butyl-[1,3,4]-oxadiazol-5-yl, 2-phenyl-[1,3,4]-oxadiazol-5-yl, 2-methoxymethyl-[1,3,4]-oxadiazol-5-yl, 2-furan-2-yl-[1,3,4]-oxadiazol-5-yl, 2-thien-2-yl-[1,3,4]-oxadiazol-5-yl, 2-(4-methoxyphenyl)-[1,3,4]-oxadiazol-5-yl, 2-(2-methoxyphenyl)-[1,3,4]-oxadiazol-5-yl, 2-(3-methoxyphenyl)-[1,3,4]-oxadiazol-5-yl, 2-(2-trifluoromethoxyphenyl)-[1,3,4]-oxadiazol-5-yl, 2-(3-trifluoromethoxy-phenyl)-[1,3,4]-oxadiazol-5-yl, 2-(4-trifluoromethoxyphenyl)-[1,3,4]-oxadiazol-5-yl, 2-(4-dimethylaminophenyl)-[1,3,4]-oxadiazol-5-yl, pyradizin-3-yl, pyrimidin-2-yl, 3-phenyl-[1,2,4]-oxadiazol-5-yl, 3-ethyl-[1,2,4]-oxadiazol-5-yl, 3-cyclopropyl-[1,2,4]-oxadiazol-5-yl, 3-thien-3-yl-[1,2,4]-oxadiazol-5-yl, 3-pyridin-4-yl-[1,2,4]-oxadiazol-5-yl, 3-pyridin-2-yl-[1,2,4]-oxadiazol-5-yl, 5-ethyl-[1,2,4]-oxadiazol-3-yl, 5-phenyl-[1,2,4]-oxadiazol-3-yl, 5-thien-3-yl-[1,2,4]-oxadiazol-3-yl, 5-trifluoromethyl-[1,2,4]-oxadiazol-3-yl, 5-pyridin-4-yl-[1,2,4]-oxadiazol-3-yl, or 5-phenyloxazol-2-yl. Even more preferably, R¹⁰ is benzoxazol-2-yl, oxazolo[4,5-b]pyridin-2-yl, 2-ethyl-[1,3,4]-oxadiazol-5-yl, 2-phenyl-[1,3,4]-oxadiazol-5-yl, 3-phenyl-[1,2,4]-oxadiazol-5-yl, 3-thien-3-yl-[1,2,4]-oxadiazol-5-yl, 3-pyridin-3-yl-[1,2,4]-oxadiazol-5-yl, 3-ethyl-[1,2,4]-oxadiazol-5-yl, 5-ethyl-[1,2,4]-oxadiazol-3-yl, or 2-methoxymethyl-[1,3,4]-oxadiazol-5-yl. Most preferably R¹⁰ is benzoxazol-2-yl.

B. Another preferred group of compounds is that wherein E is —C(R⁵)(R⁶)X¹ in which R⁵ and R⁶ taken together with the carbon atom to which both R⁵ and R⁶ are attached form cycloalkylene or heterocycloalkylene, preferably cyclopropylene, cyclopentylene, cyclohexylene, tetrahydropyran-4-yl, tetrahydrothiopyran-4-yl, tetrahydrothiopyran-4-yl-1-oxide, tetrahydrothiopyran-4-yl-1,1-dioxide, or piperidin-4-yl wherein the nitrogen atom is optionally substituted with alkyl, alkoxy, or hydroxy, preferably tetrahydrothiopyran-4-yl-1,1-dioxide, and X¹ is —CHO, —C(O)R¹⁰, —C(O)CF₃, —C(O)CF₂CF₂R⁹, —CH═CHS(O)₂R¹⁰, —C(O)CF₂C(O)NR¹⁰R¹¹, —C(O)C(O)NR¹⁰R¹¹, —C(O)CH₂OR¹⁰, —C(O)CH₂N(R¹)SO₂R¹⁰, —C(O)C(O)N(R¹¹)(CH₂)₂OR¹¹, —C(O)C(O)N(R¹¹)(CH₂)₂NR¹¹ or —C(O)C(O)R¹⁰. More preferably, X¹ is —C(O)C(O)NR¹⁰R¹¹ where R¹¹ is hydrogen and R¹⁰ is cycloalkyl or benzyl. Preferably, R¹⁰ is cyclopropyl and R¹¹ is hydrogen.
C. Yet another preferred group of compounds is that wherein E is a group of formula (a): 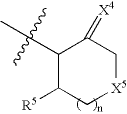
in which:

n is 0, 1, or 2, X⁴ is —NR²²—, —O— or —S— where R²² is hydrogen, alkyl, or alkoxy; X⁵ is —O—, —S(O)₂—, —S— or —NR²³ where R²³ is selected from hydrogen, alkyl, —S(O)₂R²⁴, —C(O)OR²⁶, or acyl,—where R²⁴ is alkyl, haloalkyl, cycloalkyl, cycloalkylalkyl, heterocycloalkyl, heterocycloalkylalkyl, aryl, aralkyl, heteroaryl, or heteroaralkyl and R²⁶ is hydrogen or alkyl. Preferably, X⁴ is —O—, n is 0 or 1, and X⁵ is —O—.

D. Yet another preferred group of compounds is that wherein E is —CR^{5a}R^{6a}CN wherein R^{5a} and R^{6a} are hydrogen.

E. Yet another preferred group of compounds is that wherein E is —CR^{5a}R^{6a}CN wherein R^{5a} and R^{6a} together with the carbon atom to which they are attached form cycloalkylene optionally substituted with one or two R^{b} independently selected from alkyl, halo, dialkylamino, aryl, aralkyl, cycloalkyl, cycloalkylalkyl, heteroaryl, heteroaralkyl, alkoxycarbonyl, or aryloxycarbonyl. Preferably, R^{5a} and R^{6a} together with the carbon atom to which they are attached form cyclopropylene, cyclobutylene, cyclopentylene, or cyclohexylene optionally substituted with groups described immediately above. More preferably, R^{5a} and R^{6a} together with the carbon atom to which they are attached form cyclopropylene, cyclobutylene, cyclopentylene, cyclohexylene, cycloheptylene, 2-methylcyclopropylene, 3-benzylcyclo-pentylene, 3-cyclohexylmethylcyclopentylene, 3-cyclopentylmethylcyclopentylene, 3-phenylcyclopentylene, 3-cyclohexylcyclopentylene, 3-cyclopentylcyclopentylene, 3-pyridin-2-ylmethylcyclopentylene, 3-pyridin-3-ylmethylcyclopentylene, 3-pyridin-4-ylmethyl-cyclopentylene, 2-methylcyclopropylene, 2,3-dimethylcyclopropylene, 3-benzylcyclobutylene, 3-methylcyclopentylene, 3,4-dimethylcyclopentylene, 3-ethylcyclopentylene, 3-(1,1-dimethylpropyl)-cyclopentylene, 3-n-butylcyclopentylene, 3-ethoxycarbonylcyclopentylene, 3,4-diethoxycarbonyl-cyclopentylene, or 3-benzyl-4-dimethylaminocyclopentylene. Most preferably, R^{5a} and R^{6a} together with the carbon atom to which they are attached form cyclopropylene.

F. Yet another preferred group of compounds is that wherein E is —CR^{5a}R^{6a}CN wherein R^{5a} and R^{6a} together with the carbon atom to which they are attached form heterocycloalkylene optionally substituted with one to four alkyl or one or two R^{c} which are independently selected from alkyl, haloalkyl, hydroxyalkyl, alkoxyalkyl, alkoxyalkyloxyalkyl, aryloxyalkyl, heteroaryloxyalkyl, aminoalkyl, acyl, aryl, aralkyl, heteroaryl, heteroaralkyl, heterocycloalkyl, heterocycloalkylalkyl, cycloalkyl, cycloalkylalkyl, —S(O)ₙ₂R¹⁴, -alkylene-S(O)ₙ₂—R¹⁵, —COOR¹⁶, -alkylene-COOR¹⁷, —CONR¹⁸R¹⁹, or -alkylene-CONR²⁰R²¹ (where n2 is 0-2 and R¹⁴-R¹⁷, R¹⁸ and R²⁰ are independently hydrogen, alkyl, haloalkyl, aryl, aralkyl, heteroaryl, heteroaralkyl, cycloalkyl, cycloalkylalkyl, or heterocycloalkyl and R¹⁹ and R²¹ are independently hydrogen or alkyl) wherein the aromatic or alicyclic ring in the groups attached to heterocycloalkylene is optionally substituted with one, two, or three substituents independently selected from alkyl, haloalkyl, cycloalkyl, cycloalkylalkyl, benzyl, alkoxy, hydroxy, haloalkoxy, halo, carboxy, alkoxycarbonyl, amino, monsubstituted amino, disubstituted amino, or acyl. Preferably, R^{5a} and R^{6a} together with the carbon atom to which they are attached form pyrrolidinyl, piperidinyl, tetrahydropyranyl, tetrahydrothiopyranyl, tetrahydrofuranyl, tetrahydrothiopyran-4-yl-1-oxide, tetrahydrothiopyran-4-yl-1,1-dioxide, hexahydropyridmidinyl, or hexahydropyridazinyl optionally substituted as described above. More preferably, R^{5a} and R^{6a} together with the carbon atom to which they are attached form piperidin-4-yl substituted with one to three alkyl and one R^{c} selected from haloalkyl, aminoalkyl, alkoxycarbonyl, alkoxyalkyl, alkoxyalkyloxyalkyl, heterocycloalkyl, heterocycloalkylalkyl, -alkylene-CONR²⁰R²¹, or cycloalkyl wherein the alicyclic ring is optionally substituted with substitutents listed above. Most preferably, R^{5a} and R^{6a} together with the carbon atom to which they are attached form piperidin-4-yl optionally substituted at the 1-position with methyl, ethyl, propyl, n-butyl, n-pentyl, 3-dimethylaminopropyl, 4-dimethylaminobutyl, 3-morpholin-4-ylpropyl, 3-piperidin-1-yl-propyl, 3-(4-methylpiperazin-1-yl)propyl, 3-(1-methylpiperidin-4-yl)propyl, 4-morpholin-4-ylbutyl, 2-(2-methoxyethyloxy)ethyl, 4-methoxybutyl, 4-aminocarbonylbutyl, 3-aminocarbonylpropyl, morpholin-4-yl, 4-methylpiperazin-1-yl, 1-ethoxycarbonylpiperidin-4-yl, 1,1-dioxotetrahydrothiopyran-4-yl, hydroxy, 2,2,2-trifluoroethyl, or tert-butyl, 1,2-dimethylpiperidin-4-yl, 1,2,6-trimethylpiperidin-4-yl, 1,2,2-trimethylpiperidin-4-yl, 1-methyl-2-oxopiperidin-4-yl, 1-methylpiperidin-3-yl, 1-tert-butoxycarbonylpiperidin-4-yl, 1-cyclohexylpiperidin-4-yl, 1-cyclopropylmethylpyrrolidin-3-yl, 1-benzylpyrrolidin-3-yl, 1-benzyloxycarbonylpyrrolidin-3-yl, pyrrolidin-3-yl, 1-hydroxypyrrolidin-3-yl, 1-methylpyrrolidin-3-yl, 1-ethypyrrolidin-3-yl, 1-n-propyl or n-butylpyrrolidin-3-yl, 1-cyclohexylpyrrolidin-3-yl, 1-ethyl-2,2-dimethylpyrrolidin-4-yl, 1-propyl-2-methoxycarbonylpiperidin-4-yl, 2-oxopyrrolidin-3-yl, 1-ethyl-2-oxopyrrolidin-3-yl, morpholin-4-yl, 1-(1-methylpiperidin-4-ylcarbonyl)piperidin-4-yl, 1-ethoxycarbonylpiperidin-4-yl, 1-benzylazetidin-3-yl, tetrahydrothiopyran-4-yl-1-oxide, or tetrahydrothiopyran-4-yl-1,1-dioxide. Particularly preferably, R^{5a} and R^{6a} together with the carbon atom to which they are attached form piperidin-4-yl substituted at the 1-position with ethyl, n- or 2-propyl, tetrahydrothiopyran-4-yl tetrahydrothiopyran-4-yl-1-oxide, or tetrahydrothiopyran-4-yl-1,1-dioxide. Even more particularly preferably, R^{5a} and R^{6a} together with the carbon atom to which they are attached form piperidin-4-yl substituted at the 1-position with ethyl, n- or 2-propyl or tetrahydrothiopyran-4-yl-1,1-dioxide.

I. Within the above preferred and more preferred groups (A-F), an even more preferred group of compounds is that wherein R¹ and R² are hydrogen.

(i) Within these preferred, more preferred, and even more preferred groups, a more preferred group of compounds is that wherein Q is —CO—.

(ii). Within these preferred, more preferred, and even more preferred groups, another more preferred group of compounds is that wherein Q is —OCO—.

(iii). Within these preferred, more preferred, and even more preferred groups, yet another more preferred group of compounds is that wherein Q is —NHCO—.

(iv). Within these preferred, more preferred, and even more preferred groups, yet another more preferred group of compounds is that wherein Q is —CH(CF₃)—.

Within the above preferred, more preferred, and even more preferred groups above, a particularly preferred group of compounds is that wherein:

(a) R^{1a} is -(alkylene)-SiR³²R³³R³⁴ where R³² is alkyl, R³³ is alkyl, and is alkyl. Preferably, R³², R³³, and R³⁴ are independently methyl, ethyl, n-propyl, isopropyl, butyl, sec-butyl, or tert-butyl. More preferably, R^{1a} is —CH₂—Si(CH₃)₃, —CH₂—Si(2-methylpropyl)(CH₃)₂, —CH₂—Si(2-tert-butyl)(CH₃)₂, or —(CH₂)₂—Si(ethyl)(CH₃)₂. Even more preferably, R^{1a} is —CH₂—Si(CH₃)₃.

(b) Within the above preferred, more preferred, and even more preferred groups above, another particularly preferred group of compounds is that wherein:

R^{1a} is a group having the structure: 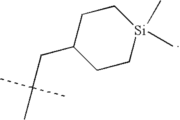
(c) Within the above preferred, more preferred, and even more preferred groups above, another particularly preferred group of compounds is that wherein:

R^{1a} is -(alkylene)-SiR³²R³³R³⁴ where R³² is alkyl and R³³ and R³⁴ together with Si form a heterocycloalkylene ring containing a Si atom and 4 or 5 carbon ring atoms wherein one or two carbon ring atoms are optionally independently replaced with —NH—, —O—, —S—, —SO—, —SO₂—, —CO—, —CONH—, or —SO₂NH—. Preferably, R^{1a} is a group having the structure:

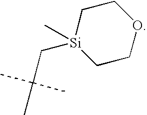

Preferably, R^{1a} is a group having the structure: 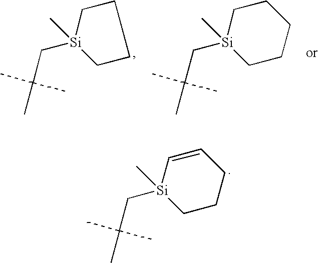
(d) Within the above preferred, more preferred, and even more preferred groups above, another particularly preferred group of compounds is that wherein:

R^{1a} is -(alkylene)-SiR³²R³³R³⁴ where R³² and R³³ are alkyl and R³⁴ is cycloalkylalkyl. Preferably, R^{1a} is a group having the structure: 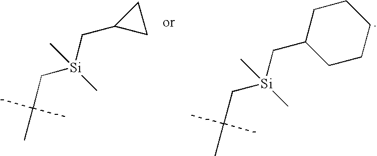
(e) Within the above preferred, more preferred, and even more preferred groups above, another particularly preferred group of compounds is that wherein:

R^{1a} is -(alkylene)-SiR³²R³³R³⁴ where R³² and R³³ are alkyl and R³⁴ is aralkyl. Preferably, R^{1a} is a group having the structure: 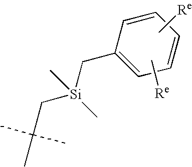
where each R^{e} is independently selected from hydrogen, alkyl, haloalkyl, haloalkoxy, or alkoxy.
(f) Within the above preferred, more preferred, and even more preferred groups above, yet another particularly preferred group of compounds is that wherein:

R^{1a} is -(alkylene)-SiR³²R³³R³⁴ where R³² and R³³ are alkyl and R³⁴ is heteroaralkyl optionally substituted with R^{e}. Preferably, R^{1a} is a group having the structure: 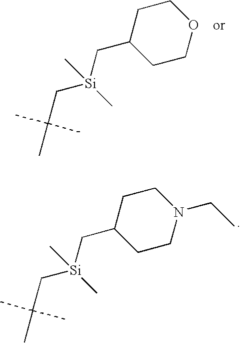
(g) Within the above preferred, more preferred, and even more preferred groups above, yet another particularly preferred group of compounds is that wherein:

R^{1a} is -(alkylene)-SiR³²R³³R³⁴ where R³² and R³³ are alkyl and R³⁴ is aryl. Preferably, R^{1a} is a group having the structure: 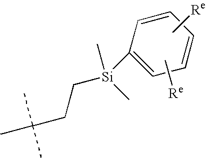
where each R^{e} is independently selected from hydrogen, alkyl, haloalkyl, haloalkoxy, or alkoxy.

Within the above preferred, more preferred, even more preferred, and particularly preferred group of compounds, a more particularly preferred group is that wherein R³ is alkyl, cycloalkyl, cycloalkylalkyl, heterocycloalkyl, heterocycloalkylalkyl, aryl, aralkyl, heteroaryl or heteroaralkyl, preferably, aryl, heteroaryl, or heterocycloalkyl wherein said cycloalkyl, heterocycloalkyl, aryl or heteroaryl ring is optionally substituted with one or two R^{f}.

Within the above preferred, more preferred, even more preferred, and particularly preferred group of compounds, another more particularly preferred group is that wherein R³ is is a group selected from methyl, cyclohexylmethyl, 3-cyclohexylpropyl, 2-cyclohexylethyl, 2-cyclopentylethyl, 6-hydroxypyrid-3-yl, 1H-imidazol-4-yl, morpholin-4-yl, naphth-1-ylmethyl, 2-phenylethyl, piperazin-1-yl, piperidin-4-yl, pyrazin-2-yl, pyridin-3-yl, pyridin-4-yl, and tetrahydropyran-4-yl.

Within the above preferred, more preferred, even more preferred, and particularly preferred group of compounds, yet another more particularly preferred group is that wherein Q is —CO— and R is morpholin-4-yl, piperidin-4-yl, pyrazin-2-yl, pyridin-3-yl, pyridin-4-yl, or tetrahydropyran-4-yl.

Within the above preferred, more preferred, even more preferred, and particularly preferred group of compounds, yet another more particularly preferred group is that wherein Q is —CHCF₃— and R³ is aryl optionally substituted with one, two, or three R^{f} independently selected from alkyl, halo, hydroxyl, alkoxy, haloalkyl, haloalkoxy, or carboxy. Preferably, R³ is phenyl, 4-fluorophenyl, 2,3-difluorophenyl, 2,4-difluorophenyl, or 2,6-difluorophenyl. More preferably, R³ is phenyl, 4-fluorophenyl, or 2,6-difluorophenyl.

G. Another preferred group of compounds of Formula (I) is that wherein:

R^{1a} is -(alkylene)-SiR³²R³³R³⁴ where R³² is alkyl, R³³ is alkyl, and R³⁴ is alkyl. Preferably,

R³², R³³, and R³⁴ are independently methyl, ethyl, n-propyl, isopropyl, butyl, sec-butyl, or tert-butyl. More preferably, R^{1a} is —CH₂—Si(CH₃)₃ or —CH₂—Si(2-methylpropyl)(CH₃)₂. Even more preferably, R^{1a} is —CH₂—Si(CH₃)₃.

Within this group, a more preferred group of compounds is that wherein:

Q is —CO—; and

R¹ and R² are hydrogen.

H. Another preferred group of compounds of Formula (I) is that wherein:

R^{1a} is a group having the structure:

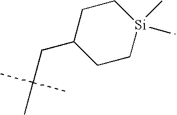

Within this group, a more preferred group of compounds is that wherein:

Q is —CO—; and

R¹ and R² are hydrogen.

I. Another preferred group of compounds of Formula (I) is that wherein:

R^{1a} is -(alkylene)-SiR³²R³³R³⁴ where R³² is alkyl and R³³ and R³⁴ together with Si form a heterocycloalkylene ring containing a Si atom and 4 or 5 carbon ring atoms wherein one or two carbon ring atoms are optionally independently replaced with —NH—, —O—, —S—, —SO—, —SO₂—, —CO—, —CONH—, or —SO₂NH—. Preferably, R^{1a} is a group having the structure:

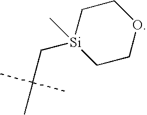

Within this group, a more preferred group of compounds is that wherein:

Q is —CO—; and

R¹ and R² are hydrogen.

J. Another preferred group of compounds of Formula (I) is that wherein:

R^{1a} is -(alkylene)-SiR³²R³³R³⁴ where R³² is alkyl and R³³ and R³⁴ together with Si form a heterocycloalkylene ring. Preferably, R^{1a} is a group having the structure:

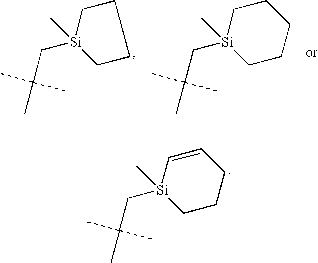

Within this group, a more preferred group of compounds is that wherein:

Q is —O—; and

R¹ and R² are hydrogen.

K. Another preferred group of compounds of Formula (I) is that wherein:

R^{1a} is -(alkylene)-SiR³²R³³R³⁴ where R³² and R³³ are alkyl and R³⁴ is cycloalkylalkyl. Preferably, R^{1a} is a group having the structure:

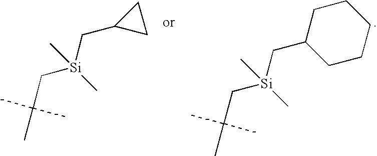

Within this group, a more preferred group of compounds is that wherein:

Q is —CO—; and

R¹ and R² are hydrogen.

L. Another preferred group of compounds of Formula (I) is that wherein:

R^{1a} is -(alkylene)-SiR³²R³³R³⁴ where R³² and R³³ are alkyl and R³⁴ is aralkyl. Preferably, R^{1a} is a group having the structure: 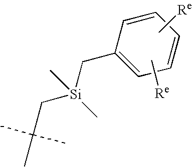
where each R^{e} is independently selected from hydrogen, alkyl, haloalkyl, haloalkoxy, or alkoxy.

Within this group, a more preferred group of compounds is that wherein:

Q is CO—; and

R¹ and R² are hydrogen.

M. Another preferred group of compounds of Formula (I) is that wherein:

R^{1a} is -(alkylene)-SiR³²R³³R³⁴ where R³² and R³³ are alkyl and R³⁴ is heteroaralkyl optionally substituted with R^{e}. Preferably, R^{1a} is a group having the structure: 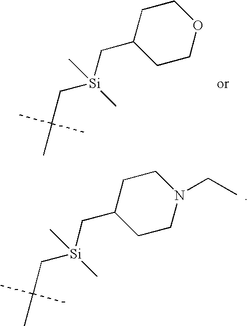
Within this group, a more preferred group of compounds is that wherein:

Q is —CO—; and

R¹ and R² are hydrogen.

N. Another preferred group of compounds of Formula (I) is that wherein:

R^{1a} is -(alkylene)-SiR³²R³³R³⁴ where R³² and R³³ are alkyl and R³⁴ is aryl. Preferably, R^{1a} is a group having the structure: 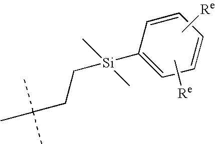
where each R^{e} is independently selected from hydrogen, alkyl, haloalkyl, haloalkoxy, or alkoxy.

Within this group, a more preferred group of compounds is that wherein:

Q is —CO—; and

R¹ and R² are hydrogen.

Within the above preferred and more preferred groups in (G-N), an even more preferred group of compounds is that wherein E is —CHR⁶C(O)R¹⁰ where R⁶ is alkyl, preferably ethyl, propyl, or butyl, more preferably ethyl, and R¹⁰ is heteroaryl optionally substituted with one or two R^{d} independently selected from alkyl, haloalkyl, alkoxy, cycloalkyl, hydroxy, haloalkoxy, halo, carboxy, alkoxycarbonyl, aryl, heteroaryl, amino, monsubstituted amino, disubstituted amino, or acyl wherein the aromatic or alicyclic ring in R^{d} is optionally substituted with one, two, or three substitutents independently selected from alkyl, haloalkyl, alkoxy, haloalkoxy, halo, hydroxy, carboxy, alkoxycarbonyl, amino, alkylamino, or dialkylamino, more preferably R¹⁰ is benzoxazol-2-yl, 4-azabenzoxazol-2-yl, 2-pyridin-3-yl-[1,3,4]-oxadiazol-5-yl, 2-pyridin-4-yl-[1,3,4]-oxadiazol-5-yl, 2-ethyl-[1,3,4]-oxadiazol-5-yl, 2-isopropyl-[1,3,4]-oxadiazol-5-yl, 2-tert-butyl-[1,3,4]-oxadiazol-5-yl, 2-phenyl-[1,3,4]-oxadiazol-5-yl, 2-methoxymethyl-[1,3,4]-oxadiazol-5-yl, 2-furan-2-yl-[1,3,4]-oxadiazol-5-yl, 2-thien-2-yl-[1,3,4]-oxadiazol-5-yl, 2-(4-methoxy-phenyl)-[1,3,4]-oxadiazol-5-yl, 2-(2-methoxyphenyl)-[1,3,4]-oxadiazol-5-yl, 2-(3-methoxy-phenyl)-[1,3,4]-oxadiazol-5-yl, 2-(2-trifluoromethoxyphenyl)-[1,3,4]-oxadiazol-5-yl, 2-(3-trifluoromethoxyphenyl)-[1,3,4]-oxadiazol-5-yl, 2-(4-trifluoromethoxyphenyl)-[1,3,4]-oxadiazol-5-yl, 2-(4-dimethylaminophenyl)-[1,3,4]-oxadiazol-5-yl, pyradizin-3-yl, pyrimidin-2-yl, 3-phenyl-[1,2,4]-oxadiazol-5-yl, 3-ethyl-[1,2,4]-oxadiazol-5-yl, 3-cyclopropyl-[1,2,4]-oxadiazol-5-yl, 3-thien-3-yl-[1,2,4]-oxadiazol-5-yl, 3-pyridin-4-yl-[1,2,4]-oxadiazol-5-yl, 3-pyridin-2-yl-[1,2,4]-oxadiazol-5-yl, 5-ethyl-[1,2,4]-oxadiazol-3-yl, 5-phenyl-[1,2,4]-oxadiazol-3-yl, 5-thien-3-yl-[1,2,4]-oxadiazol-3-yl, 5-trifluoromethyl-[1,2,4]-oxadiazol-3-yl, 5-pyridin-4-yl-[1,2,4]-oxadiazol-3-yl, or 5-phenyloxazol-2-yl. Even more preferably, R¹⁰ is benzoxazol-2-yl, oxazolo[4,5-b]pyridin-2-yl, 2-ethyl-[1,3,4]-oxadiazol-5-yl, 2-phenyl-[1,3,4]-oxadiazol-5-yl, 3-phenyl-[1,2,4]-oxadiazol-5-yl, 3-thien-3-yl-[1,2,4]-oxadiazol-5-yl, 3-pyridin-3-yl-[1,2,4]-oxadiazol-5-yl, 3-ethyl-[1,2,4]-oxadiazol-5-yl, 5-ethyl-[1,2,4]-oxadiazol-3-yl, or 2-methoxymethyl-[1,3,4]-oxadiazol-5-yl.

Within the above preferred and more preferred groups in (G-N), another even more preferred group of compounds is that wherein E is —CR^{5a}R^{6a}CN wherein R^{5a} and R^{6a} together with the carbon atom to which they are attached form cycloalkylene, preferably cyclopropylene.

Within the above preferred and more preferred groups in (G-N), another even more preferred group of compounds is that wherein E is —CR^{5a}R^{6a}CN wherein R^{5a} and R^{6a} together with the carbon atom to which they are attached form heterocycloalkylene, preferably R^{5a} and R^{6a} together with the carbon atom to which they are attached form piperidin-4-yl substituted at the 1-position with ethyl, n- or 2-propyl, tetrahydrothiopyran-4-yl tetrahydrothiopyran-4-yl-1-oxide, or tetrahydrothiopyran-4-yl-1,1-dioxide.

Within the above preferred and more preferred groups in (G-N), another even more preferred group of compounds is that wherein E is —CR⁶COCOR¹⁰ where R¹⁰ is cycloalkyl, preferably R⁶ is ethyl, propyl, or butyl and R¹⁰ is cyclopropyl.

Within the above preferred, more preferred group, and even more preferred groups, a particularly preferred group of compounds is that wherein R³ is aryl, heteroaryl, or heterocycloalkyl. Preferably, R³ is morpholin-4-yl, 1-ethylpiperazin-4-yl, phenyl optionally substituted with one or two substitutents independently selected from halo, alkoxy, alkyl, haloalkoxy, phenyl, alkylsulfonyl, haloalkyl, heteroaryl, cyano, acyl, hydroxyalkyl, or alkoxycarbonyl. Preferably, R³ is morpholin-4-yl, 1-ethylpiperazin-4-yl, 3′-methoxybiphen-3-yl, 3′-iodophenyl, 3′-trifluoromethoxybiphen-3-yl, biphen-3-yl, 2′,6′-dimethoxybiphen-3-yl, 4′-methylsulfonyl-biphen-3-yl, 2′-chlorobiphen-3-yl, 2′-trifluoromethylbiphen-3-yl, 3′-methylbiphen-3-yl, 3-pyridin-3-yl-phenyl, 3′-cyanobiphen-3-yl, 3′-hydroxymethylbiphen-3-yl, 4′-hydroxymethyl-biphen-3-yl, 2′-methylbiphen-3-yl, 3′-methoxycarbonylbiphen-3-yl, or 4′-acetylbiphen-3-yl.

Additionally, in the preferred embodiments above, a number of different preferences have been given above, and following any one of these preferences results in a compound of this invention that is more presently preferred than a compound in which that particular preference is not followed. However, these preferences are generally independent; and following more than one of these preferences may result in a more presently preferred compound than one in which fewer of the preferences are followed.

I. Compounds of Formula (I) where Q is —CO—, R¹, R² are hydrogen, E is CR⁵R⁶CR⁷R⁸R¹⁰ where R⁵ is hydrogen, R⁷ and R⁸ together form oxo and R³, R^{1a}, R⁶ and R¹⁰ are as defined below are:

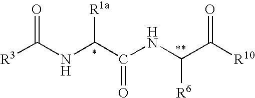

| | Stereochem | | | | |
|---|---|---|---|---|---|
| Cpd. # | (*C, **C) | R³ | R^{1a} | R⁶ | R¹⁰ |
| 1 | R, S | morpholin-4-yl | —CH₂Si(CH₃)₃ | n-propyl | benzoxazol-2-yl |
| 2 | R, S | morpholin-4-yl | —CH₂Si(CH₃)₃ | ethyl | benzoxazol-2-yl |
| 3 | R, R | morpholin-4-yl | —CH₂Si(CH₃)₃ | ethyl | benzoxazol-2-yl |
| 4 | R, S | morpholin-4-yl | —CH₂Si(CH₃)₃ | n-butyl | benzoxazol-2-yl |
| 5 | R, S | morpholin-4-yl | —CH₂Si(CH₃)₃ | ethyl | 5-Cl-benzoxazol-2-yl |
| 6 | S, S | morpholin-4-yl | —CH₂Si(CH₃)₃ | ethyl | benzoxazol-2-yl |
| 7 | S, S | morpholin-4-yl | —CH₂Si(CH₃)₃ | n-propyl | benzoxazol-2-yl |

and are named as:
morpholine-4-carboxylic acid {1(R)-[1(S)-(benzoxazol-2-ylcarbonyl)-butylcarbamoyl]-2-trimethylsilanylethyl}amide;
morpholine-4-carboxylic acid {1(R)-[(S)-(benzoxazol-2-ylcarbonyl)-propylcarbamoyl]-2-trimethylsilanylethyl}amide; 27883 ¹H-NMR(CDCl₃): 7.86(d, J=8 Hz, 1H), 7.62(d, J=8 Hz, 1H), 7.51(dt, J=7.2 Hz, J=1.2 Hz, 1H), 7.43(dt, J=8 Hz, J=1.2 Hz, 1H), 6.94 (d, J=7.2 Hz, 1H), 5.55 (m, 1H), 4.83(d, J=8.0 Hz, 1H), 4.42(m, 1H), 3.65(m, 4H), 3.31(m, 4H), 2.13(m, 1H), 1.87(m, 1H), 1.14(m, 2H), 0.98(t, J=12 Hz, 2H), 0.0(s, 9H). LC-MS: 459.2(M−1), 461.3(M+1). Exact mass: 460.21
morpholine-4-carboxylic acid {1(R)-[1(R)-(benzoxazol-2-ylcarbonyl)-propylcarbamoyl]-2-trimethylsilanylethyl}amide;
morpholine-4-carboxylic acid {1(R)-[1(S)-(benzoxazol-2-ylcarbonyl)-pentylcarbamoyl]-2-trimethylsilanylethyl}amide;
morpholine-4-carboxylic acid {1(R)-[1(S)-(5-chlorobenzoxazol-2-ylcarbonyl)-propylcarbamoyl]-2-trimethylsilanylethyl}amide; ¹H-NMR(CDCl₃): 7.83(d, J=2 Hz, 1H), 7.54(d, J=8.8 Hz, 1H), 7.46(dd, J=8.8 Hz, J=2 Hz, 1H), 6.93(d, J=6.4 Hz, 1H), 5.49(m, 1H), 4.77(d, J=8 Hz, 1H), 4.41 (m, 1H), 3.60(m, 4H), 3.32(m, 4H), 2.13(m, 1H), 1.84(m, 1H), 1.16(m, 2H), 0.97(t, J=11.6 Hz, 2H), 0.98(m, 1H), 0.007(s, 9H). LC-MS: 493.2 (M−1), 495.4(M+1). Exact mass: 494.18
morpholine-4-carboxylic acid {1(S)-[1(S)-(benzoxazol-2-ylcarbonyl)-propylcarbamoyl]-2-trimethylsilanylethyl}amide; LCMS: 461.1 (M+1)⁺¹, 483.0(M+Na)⁺, 459.1(M−1)⁻¹; and
morpholine-4-carboxylic acid {1(S)-[1(S)-(benzoxazol-2-ylcarbonyl)-butylcarbamoyl]-2-trimethylsilanylethyl}amide LCMS: 475.1(M+1)⁺¹, 497.0 (M+Na)⁺, 473.2(M−1)⁻¹.

II. Compounds of Formula (I) where Q is —CO—, R¹, R² are hydrogen, E is CR^{5a}R^{6a}CN where R^{5a} and R^{6a} are as defined below and R³ and R^{1a} are as defined below are:

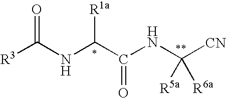

| | Stereochem | | | | | |
|---|---|---|---|---|---|---|
| Cpd. # | (*C) | R³ | R^{1a} | R^{5a} | R^{6a} | R^{5a} + R^{6a} |
| 1 | R | morpholin-4-yl | —CH₂Si(CH₃)₃ | — | — | cyclopropyl |
| 2 | R | morpholin-4-yl | —CH₂Si(CH₃)₃ | — | — | 1-ethylpiperidin-4-yl |
| 3 | R | morpholin-4-yl | —CH₂Si(CH₃)₃ | — | — | 1,1-dioxohexahydro-1λ⁶- |
| | | | | | | thiopyran-4-yl |
| 4 | RS | morpholin-4-yl | —CH₂Si(CH₃)₃ | ethyl | benzyloxymethyl | — |
| 5 | RS | morpholin-4-yl | —CH₂Si(CH₃)₃ | methyl | benzyloxymethyl | — |
| 6 | R | 4-ethylpiperazin-1-yl | —CH₂Si(CH₃)₃ | — | — | cyclopropyl |
| 7 | R | 3′-methoxybiphen-3-yl | —CH₂Si(CH₃)₃ | — | — | cyclopropyl |
| 8 | RS | 3′-iodophenyl | —CH₂Si(CH₃)₃ | — | — | cyclopropyl |
| 9 | RS | 3′-trifluoromethoxy- | —CH₂Si(CH₃)₃ | — | — | cyclopropyl |
| | | biphen-3-yl | | | | |
| 10 | RS | biphen-3-yl | —CH₂Si(CH₃)₃ | — | — | cyclopropyl |
| 11 | RS | 2′,6′-dimethoxy- | —CH₂Si(CH₃)₃ | — | — | cyclopropyl |
| | | biphen-3-yl | | | | |
| 12 | RS | 4′-methylsulfonyl- | —CH₂Si(CH₃)₃ | — | — | cyclopropyl |
| | | biphen-3-yl | | | | |
| 13 | RS | 2′-chlorobiphen-3-yl | —CH₂Si(CH₃)₃ | — | — | cyclopropyl |
| 14 | RS | 2′-trifluoromethyl- | —CH₂Si(CH₃)₃ | — | — | cyclopropyl |
| | | biphen-3-yl | | | | |
| 15 | RS | 3′-methylbiphen-3-yl | —CH₂Si(CH₃)₃ | — | — | cyclopropyl |
| 16 | RS | 3-pyridin-3-yl-phenyl | —CH₂Si(CH₃)₃ | — | — | cyclopropyl |
| 17 | RS | 3′-cyanobiphen-3-yl | —CH₂Si(CH₃)₃ | — | — | cyclopropyl |
| 18 | RS | 3′-hydroxymethyl- | —CH₂Si(CH₃)₃ | — | — | cyclopropyl |
| | | biphen-3-yl | | | | |
| 18 | RS | 4′-hydroxymethyl- | —CH₂Si(CH₃)₃ | — | — | cyclopropyl |
| | | biphen-3-yl | | | | |
| 20 | RS | 2′-methylbiphen-3-yl | —CH₂Si(CH₃)₃ | — | — | cyclopropyl |
| 21 | RS | 3′-methoxycarbonyl- | —CH₂Si(CH₃)₃ | — | — | cyclopropyl |
| | | biphen-3-yl | | | | |
| 22 | RS | 4′-acetylbiphen-3-yl | —CH₂Si(CH₃)₃ | — | — | cyclopropyl |
| 23 | RS | 3′-methoxybiphen-3-yl | —CH₂Si(CH₃)₃ | — | — | tetrahydrothiopyran-4-yl |
| 24 | RS | 3′-methoxybiphen-3-yl | —CH₂Si(CH₃)₃ | — | — | 1,1-dioxohexahydro-1λ⁶- |
| | | | | | | thiopyran-4-yl |

and are named as:
1-(R)-morpholine-4-carboxylic acid [1-(1-cyanocyclopropylcarbamoyl)-2-(trimethyl-silanyl)ethyl]amide;
1-(R)-morpholine-4-carboxylic acid [1-(4-cyano-1-ethylpiperidin-4-ylcarbamoyl)-2-(trimethyl-silanyl)ethyl]amide;
1-(R)-morpholine-4-carboxylic acid [1-(4-cyano-1,1-dioxohexahydro-1λ⁶-thiopyran-4-yl-carbamoyl)-2-(trimethylsilanyl)ethyl]amide;
morpholine-4-carboxylic acid [1-(RS)-(1-benzyloxymethyl-1-cyanopropylcarbamoyl)-2-trimethyl-silanylethyl]-amide;
morpholine-4-carboxylic acid [1-(RS)-(2-benzyloxy-1-cyano-1-methyl-ethylcarbamoyl)-2-trimethylsilanylethyl]amide;
4-ethylpiperazine-1-carboxylic acid [1-(R)-(1-cyanocyclopropylcarbamoyl)-2-trimethylsilanyl-ethyl]amide;
3′-methoxybiphenyl-3-carboxylic acid [1-(R)-(1-cyano-cyclopropylcarbamoyl)-2-trimethyl-silanylethyl]amide ¹H-NMR(CDCl₃): 7.89(m, 1H), 7.66(m, 2H), 7.44(t, J=4.4 Hz, 1H), 7.30(t, J=3.6 Hz, 1H), 7.11(d, J=5.6 Hz, 1H), 7.04(t, J=2 Hz, 1H), 6.85(dd, J=8.8 Hz, J=2 Hz, 1H), 4.55(q, J=8 Hz, 1H), 3.80(s, 3H), 1.43(m, 3H), 1.31(m, 1H), 1.15(m, 3H), 1.00(dd, 1H), 0.00(s, 9H). LC-MS: 434.2(M−1), 436.3(M+1). Exact mass: 435.2;
N-[1-(RS)-(1-cyanocyclopropylcarbamoyl)-2-trimethylsilanylethyl]-3-iodobenzamide M+H=456.1; M−H=454.0; exact mass=455.06;
3′-trifluoromethoxybiphenyl-3-carboxylic acid [1-(RS)-(1-cyanocyclopropylcarbamoyl)-2-trimethylsilanylethyl]amide M+H=490.2; M−H=488.2; exact mass=489.17;
biphenyl-3-carboxylic acid[1-(RS)-(1-cyanocyclopropylcarbamoyl)-2-trimethylsilanylethyl]amide M+H=406.3; M−H=404.2; Exact mass=405.19;
2′,6′-dimethoxybiphenyl-3-carboxylic acid [1-(RS)-(1-cyanocyclopropylcarbamoyl)-2-trimethylsilanylethyl]amide M+H=466.2; M−H=464.3; Exact mass=465.21
4′-methylsulfonylbiphenyl-3-carboxylic acid [1-(RS)-(1-cyanocyclopropylcarbamoyl)-2-trimethylsilanylethyl]amide ¹H-NMR(DMSO-d₆): 8.91(s, 1H), 8.71(d, J=8 Hz, 1H), 8.02(s, 5H), 7.84(m, 2H), 7.61(t, J=8 Hz, 1H), 4.5(m, 1H), 3.28(s, 3H), 1.45(m, 1H), 1.1(m, 4H), 0.01(s, 9H). LC-MS: 482.2(M−1), 484.1(M+1). Exact mass: 483.16;
2′-chlorobiphenyl-3-carboxylic acid [1-(RS)-(1-cyanocyclopropylcarbamoyl)-2-trimethyl-silanylethyl]amide M+H=440.3; M−H=438.2; Exact mass=439.16;
2′-trifluoromethylbiphenyl-3-carboxylic acid [1-(RS)-(1-cyanocyclopropylcarbamoyl)-2-trimethylsilanylethyl]amide M+H=474.3; M−H=472.3; exact mass=473.17;
3′-methylbiphenyl-3-carboxylic acid [1-(RS)-(1-cyanocyclopropylcarbamoyl)-2-trimethylsilanylethyl]amide M+H=420.5; M−H=418.3; Exact mass=419.20;
N-[1-(RS)-(1-cyanocyclopropylcarbamoyl)-2-trimethylsilanylethyl]-3-pyridin-3-ylbenzamide M+H=406.9; M−H=405.3; Exact mass=406.18;
3′-cyanobiphenyl-3-carboxylic acid [1-(RS)-(1-cyanocyclopropylcarbamoyl)-2-trimethylsilanylethyl]amide¹H-NMR(DMSO-d₆): 8.91(s, 1H), 8.69(d, J=8.4 Hz, 1H), 8.23(s, 2H), 8.11 (d, J=7.6 Hz, 1H), 7.92(m, 2H), 7.85(d, J=7.2 Hz, 1H), 7.70(t, J=8.0 Hz, 1H), 7.59(t, J=7.6 Hz, 1H), 4.50(m, 1H), 1.45(m, 2H), 1.19-1.00(m, 4H), 0.00 (s, 9H). LC-MS: 429.2(M−1), 431.3(M+1). Exact mass: 430.18;
3′-hydroxymethylbiphenyl-3-carboxylic acid [1-(RS)-(1-cyanocyclopropylcarbamoyl)-2-trimethylsilanylethyl]amide;
4′-hydroxymethylbiphenyl-3-carboxylic acid [1-(RS)-(1-cyanocyclopropylcarbamoyl)-2-trimethylsilanylethyl]amide M+Na=458.1; M−H=434.0; Exact mass=435.20;
2′-methylbiphenyl-3-carboxylic acid [1-(RS)-(1-cyanocyclopropylcarbamoyl)-2-trimethylsilanylethyl]amide M+H=420.2; Exact mass=419.20;
3′-methoxycarbonylbiphenyl-3-carboxylic acid [1-(RS)-(1-cyanocyclopropylcarbamoyl)-2-trimethylsilanylethyl]amide M+H=464.3; M−H=462.2; Exact mass=463.18;
4′-acetylbiphenyl-3-carboxylic acid [1-(RS)-(1-cyanocyclopropylcarbamoyl)-2-trimethylsilanylethyl]amide ¹H-NMR(DMSO-d₆): 8.01 (s, 1H), 7.96(d, J=7.6 Hz, 2H), 7.72(dd, J=7.6 Hz, J=1.6 Hz, 2H), 7.62(d, J=8.4 Hz, 2H), 7.5(m, 2H), 6.87(d, J=7.6 Hz, 1H), 4.6(m, 1H), 2.58(s, 3H), 1.47(m, 2H), 1.27(m, 1H), 1.18(m, 2H), 1.03(m, 1H), 0.00(s, 9H). LC-MS: 446.5 (M−1), 448.5 (M+1). Exact mass: 447;
3′-methoxybiphenyl-3-carboxylic acid [1-(RS)-(4-cyano-4-tetahydrothiopyran-4-ylcarbamoyl)-2-trimethylsilanylethyl]amide M+Na=518.5; M−H=494.5; Exact mass=495.20; and
3′-methoxybiphenyl-3-carboxylic acid [1-(RS)-(4-cyano-1,1-dioxohexahydro-1λ⁶-thiopyran-4-ylcarbamoyl)-2-(trimethylsilanyl)ethyl]-amide ¹H-NMR(CDCl₃): 7.88(m, 1H), 7.85(s, 1H), 7.70(d, J=6.8 Hz, 1H), 7.62(d, J=6.0 Hz, 1H), 7.45(t, J=6.0 Hz, 1H), 7.30(t, J=6 Hz, 1H), 7.10(d, J=7.2 Hz, 1H), 7.04(t, J=2 Hz, 1H), 6.85(d, J=6.8 Hz, 1H), 6.53(d, J=5.6 Hz, 1H), 4.55(m, 1H), 3.8(s, 2H), 3.2(m, 2H), 3.1(m, 1H), 3.0(m, 1H), 2.8(m, 1H), 2.65(m, 1H), 2.5(m, 2H), 1.33(dd, J=6.0 Hz, J=11.6 Hz, 1H), 0.96(dd, J=6.4 Hz, J=11.6 Hz, 1H), 0.80(m, 1H), 0.00(s, 9H). LC-MS: 526.4(M−1), 528.6(M+1). Exact mass: 527.20.
   III. 1-[3-(Benzyldimethylsilanyl)-2R-(2,2,2-trifluoro-1-phenylethylamino)propionyl]-cyclopropanecarbonitrile.

### General Synthetic Scheme

Compounds of this invention can be made by the methods depicted in the reaction schemes shown below.

The starting materials and reagents used in preparing these compounds are either available from commercial suppliers such as Aldrich Chemical Co., (Milwaukee, Wis.), Bachem (Torrance, Calif.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), March's Advanced Organic Chemistry, (John Wiley and Sons, 4th Edition) and Larock's Comprehensive Organic Transformations (VCH Publishers Inc., 1989). These schemes are merely illustrative of some methods by which the compounds of this invention can be synthesized, and various modifications to these schemes can be made and will be suggested to one skilled in the art having referred to this disclosure.

The starting materials and the intermediates of the reaction may be isolated and purified if desired using conventional techniques, including but not limited to filtration, distillation, crystallization, chromatography and the like. Such materials may be characterized using conventional means, including physical constants and spectral data.

Unless specified to the contrary, the reactions described herein take place at atmospheric pressure over a temperature range from about −78° C. to about 150° C., more preferably from about 0° C. to about 125° C. and most preferably at about room (or ambient) temperature, e.g., about 20° C.

In the reactions described hereinafter it may be necessary to protect reactive functional groups, for example hydroxy, amino, imino, thio or carboxy groups, where these are desired in the final product, to avoid their unwanted participation in the reactions. Conventional protecting groups may be used in accordance with standard practice, for examples see T. W. Greene and P. G. M. Wuts in “Protective Groups in Organic Chemistry” John Wiley and Sons, 1991. Compound of Formula (I) can be prepared by the procedures described in Schemes 1-4 below.

Compounds of Formula (I) where E is —C(R⁵)(R⁶)C(R⁷)(R⁸)R¹⁰ where R⁵, R⁶, R⁷, R⁸, R¹⁰ and other groups are as defined in the Summary of the Invention can be prepared by proceeding as illustrated and described in Scheme 1 below:

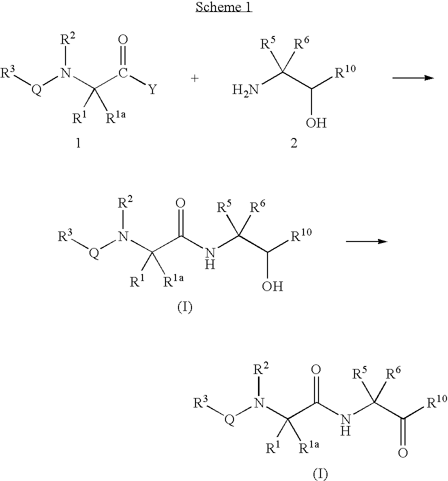

Reaction of a compound of formula 1 [where Y is hydroxy or an activating group (e.g. 2,5-dioxopyrrolidin-1-yl, succinimide, or the like), preferably hydroxy] with an aminoalcohol compound of formula 2 where R⁷ is hydrogen and R⁸ is hydroxy provides a compound of Formula (I) where R⁷ is hydrogen and R⁸ is hydroxy. The reaction conditions vary based on the nature of the Y group. When Y is an activating group, the reaction is carried out in the presence of a suitable base (e.g. triethylamine, diisopropylethylamine, or the like) and in a suitable solvent (e.g. acetonitrile, N,N-dimethylformamide (DMF), dichloromethane, or any suitable combination thereof, or the like) at 10 to 30° C., preferably at about 25° C., and requires 24 to 30 hours to complete. When Y is hydroxy, the reaction is carried out in the presence of a suitable coupling agent (e.g. benzotriazole-1-yloxytrispyrrolidinophosphonium hexafluoro-phosphate (PyBOP®), 1-(3-dimethylaminopropyl)-3-ethylcarbodiimide hydrochloride (EDC), O-benzotriazol-1-yl-N,N,N′,N′-tetramethyluronium hexafluorophosphate (HBTU), O-(7-azabenzotriazol-1-yl)-1,1,3,3-tetramethyluronium hexafluorophosphate (HATU), 1,3-dicyclohexylcarbodiimide (DCC), or the like) and a base (e.g. N,N-diisopropylethylamine, triethylamine, or the like) is required and the reaction takes about 2 to 3 hours to complete. Compounds of formula 1 and 2 are either commercially available or they can be prepared by methods well known in the art. For example, compound 1 where Q is —CO— and Y is hydroxy can be readily prepared by reacting an amino acid of formula CR¹R^{1a}(COOR′)NHR² (where R′ is hydrogen or alkyl and R¹, R² and R^{1a} are as defined in the Summary of the Invention) with an acylating agent of formula R³COL where L is a leaving group such as a halo (particularly Cl or Br) or imidazolide. Suitable solvents for the reaction include aprotic polar solvents (e.g., dichloromethane, THF, dioxane and the like.). When L is halo, the reaction is carried out in the presence of a non-nucleophilic organic base e.g., triethylamine, pyridine, and the like. Acylating agents of formula R³COL are either commercially available or they can be prepared by treating the corresponding acid with a halogenating agent such as oxalyl chloride, sulfonyl chloride, carbon tetrabromide, and the like. When R′ is alkyl, removal of the alkyl group under basic hydrolysis reaction conditions provides a corresponding compound of formula 1 where Y is hydroxy.

Compound 1 where Q is —SO₂— and Y is hydroxy can be readily prepared by reacting an amino acid of formula CR¹R^{1a}(COOR′)NHR² where R′, R¹, R² and R^{1a} are as defined above with a sulfonyl halide of the formula R³SO₂L where L is halo, utilizing the reaction conditions described in method immediately above. Sulfonyl halides are commercially available or may be prepared by methods such as those described in (1) Langer, R. F.; Can. J. Chem.; 1983, 61, 1583-1592; (2) Aveta, R.; et. al.; Gazetta Chimica Italiana, 1986, 116, 649-652; (3) King, J. F. and Hillhouse, J. H.; Can. J. Chem.; 1976, 54, 498; and (4) Szymonifka, M. J. and Heck, J. V.; Tet. Lett.; 1989, 30, 2869-2872.

Compound 1 where Q is —NHCO— and Y is hydroxy can be readily prepared by reacting an amino acid of formula CR¹R^{1a}(COOR′)NHR² where R′, R¹, R² and R^{1a} are as defined above with an activating agent such as carbonyl diimidazole/thiocarbonyl diimidazole, followed by nucleophilic displacement of the imidazole group with a primary or secondary amine of formula R³NH₂ where R³ is as defined in the Summary of the Invention. The reaction occurs at ambient temperature. Suitable solvents include polar organic solvents (e.g., THF, dioxane and the like). Alternatively, these compounds can be prepared by reacting CR¹R^{1a}(COOR′)NHR² with a carbamoyl halide of the formula R³NHCOL where L is halo. The reaction is carried out in the presence of a non-nucleophilic organic base. Suitable solvents for the reaction are dichloromethane, 1,2-dichloroethane, THF, or pyridine. These compounds can also be prepared by reacting CR¹R^{1a}(COOR′)NHR² with an isocyanate of formula R³N═C═O in an aprotic organic solvent (e.g., benzene, THF, DMF and the like).

Compound 1 where Q is —NHSO₂— and Y is hydroxy can be readily prepared by reacting an amino acid of formula CR¹R^{1a}(COOR′)NHR² where R′, R¹, R² and R^{1a} are as defined above with a sulfamoyl halide of the formula R³NHSO₂L where L is halo, utilizing the reaction conditions described in paragraph immediately above. Sulfamoyl halides are commercially available or may be prepared by methods such as those described in Graf, R; German Patent, 931225 (1952) and Catt, J. D. and Matler, W. L; J. Org. Chem., 1974, 39, 566-568.

Compound 1 where Q is —CHR— where R is haloalkyl and Y is hydroxy can be readily prepared by reacting an amino acid of formula CR¹R^{1a}(COOR′)NHR² where R′ is alkyl by the methods disclosed in PCT application Publication No. WO 03/075836, which is incorporated herein by reference in its entirety.

Amino acids of formula CR¹R^{1a}(COOR′)NHR² where R′ is hydrogen or alkyl and R¹, R^{1a} and R² are defined in the Summary of the Invention can be prepared by methods well known in the art. Detailed syntheses of an amino acid where R¹ and R² are hydrogen and R^{1a} is 2-trimethylsilylmethyl are provided in working examples below.

Compounds of formula 2 where R¹⁰ is benzoxazol-2-yl, oxazolo[4,5-b]pyridin-2-yl, and the like, can be prepared under deprotonation reaction conditions by treating benzoxazole, oxazolo[4,5-b]pyridine, 2-pyridin-3-yloxadiazole, 2-pyridin-4-yl-oxadiazole, 2-phenyloxadiazole, and the like, with a Grignard reagent such as isopropylmagnesium chloride and then reacting the resulting organomagnesium reagent with an alpha-(N-protected amino)aldehyde of formula CR⁵R⁶(NHPG)CHO, where PG is a suitable amino protecting group (such as tert-butyoxycarbonyl, benzyloxycarbonyl, or benzyl) to provide a compound of formula CR⁵R⁶(NHPG)CH(R¹⁰)OH where R¹⁰ is benzoxazol-2-yl, oxazolo[4,5-b]pyridin-2-yl, 2-pyridin-3-yloxadiazolyl, 2-pyridin-4-yl-oxadiazolyl, 2-phenyloxadiazolyl, and the like, after treatment with an aqueous acid or buffer. Removal of the amino protecting group then provides a compound of formula 2 where R¹⁰ is benzoxazol-2-yl, oxazolo[4,5-b]pyridin-2-yl, 2-pyridin-3-yloxadiazolyl, 2-pyridin-4-yl-oxadiazolyl, 2-phenyloxadiazolyl, and the like.

The addition reaction is typically carried out in an ethereal organic solvent such as tetrahydrofuran, diethyl ether, dioxane, and the like, preferably tetrahydrofuran, at a temperature from about −78° C. to about 40° C. Preferably, the reaction is carried out from about −10° C. to about 40° C., more preferably from about −10° C. to about 10° C. The reaction typically requires an hour to complete. The nucleophilic addition reaction is typically carried out from about −10° C. to about room temperature. Compounds of formula CR⁵R⁶(NHPG)CHO are prepared from commercially available amino acids by methods well known in the art. Some such methods are disclosed in working examples below.

The reaction conditions employed for removal of the amino protecting group depends on the nature of the protecting group. For example, if the protecting group is tert-butoxycarbonyl, it is removed under acid reaction conditions. Suitable acids are trifluoroacetic acid (TFA), hydrochloric acid, and the like. If the protecting group is benzyl or benzyloxycarbonyl, it is removed under catalytic hydrogenation reaction conditions. Suitable catalyst are palladium, platinum, rodium based catalysts and others known in the art. Other suitable reaction conditions for their removal can be found in Greene, T. W.; and Wuts, P. G. M.; Protecting Groups in Organic Synthesis; John Wiley & Sons, Inc. 1999. The reaction is carried out in an inert organic solvent methylene chloride, tetrahydrofuran, dioxane, dimethylformamide, and the like.

Oxidation of hydroxy group in (I) where R⁷ is hydroxy and R⁸ is hydrogen with a suitable oxidizing agent such as Dess-Martin Periodinane in a halogenated organic solvent such as methylene chloride, chloroform, carbon tetrachloride, and the like, or a mixture of TEMPO/bleach then provides a corresponding compound of Formula (I) where R⁷ and R⁸ together form oxo.

Alternatively, compounds of Formula (I) where E is C(R⁵)(R⁶)C(R⁷)(R⁸)R¹⁰ where R⁷ and R⁸ together form oxo, R⁵-R⁸, R¹⁰ and other groups are as defined in the Summary of the Invention can be prepared by proceeding as illustrated and described in Scheme 2 below:

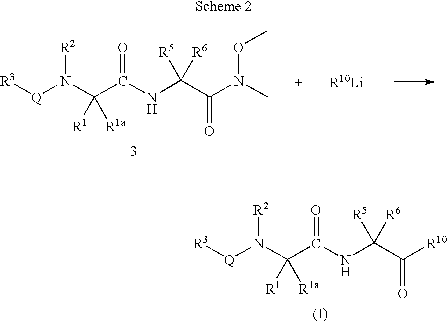

Compounds of Formula (I) where E is —C(R⁵)(R⁶)C(R⁷)(R⁸)R¹⁰ where R⁷ and R⁸ together form oxo can be prepared by reacting a compound of formula 3 with an organometallic compound of formula R¹⁰Li. The reaction is carried out in a suitable solvent (e.g. tetrahydrofuran (THF), ether, or the like) at −80 to −70° C., preferably at about −78° C., and requires 30 minutes to an hour to complete. The organometallic compound of formula R¹⁰Li is generated by treating a corresponding organo compound or a brominated derivative thereof, with n-butyllithium or tert-butyllithium in a suitable solvent (e.g. THF, ether, or the like) at −80 to −70° C., preferably at about −78° C., for approximately 30 minutes to an hour.

Compounds of formula 3 can be prepared by reacting an amino acid of formula 4 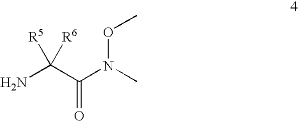
with a compound of the formula R³QN(R²)C(R¹)(R^{1a})C(O)Y where Q and R³ are as defined in the Summary of the invention and Y is hydroxy or an activating group (succinimide, or the like) under conditions described in Scheme 1 above.

Compounds formula 4 can be prepared by reacting a corresponding N-protected alpha amino acid with N,O-dimethylhydroxylamine hydrochloride followed by deprotection of the amino group. The reaction with the N,O-dimethylhydroxylamine is carried out in the presence of a suitable coupling agent (PyBOP®, EDC, HBTU, DCC, and the like) and a base (e.g. N,N-diisopropylethylamine, triethylamine, or the like) in a suitable solvent (e.g. dichloromethane, DMF, and the like) at 20 to 30° C., preferably at about 25° C., and takes about 2 to 4 hours to complete. Deprotection of the amino group provides the desired compound 4.

Compounds of Formula (I) where E is —C(R^{5a})(R^{6a})CN where R^{5a}, R^{6a} and other groups are as defined in the Summary of the Invention can be prepared by proceeding as illustrated and described in Scheme 3 below:

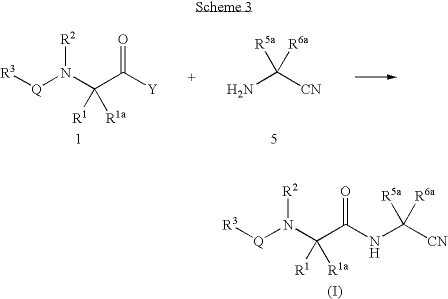

Reaction of a compound of formula 1 where Y is hydroxy or succinimide ester with an aminonitrile compound of formula 5 under the reaction conditions described in Scheme 1 above provides a compound of Formula (I). Compounds of formula 5 are either commercially available or they can be prepared by methods well known in the art.

Compounds of Formula (I) where E is —C(R⁵)(R⁶)CH═CHS(O)₂R¹⁰ where R⁵, R⁶, R¹⁰ and other groups are as defined in the Summary of the Invention can be prepared by proceeding as illustrated and described in Scheme 4 below:

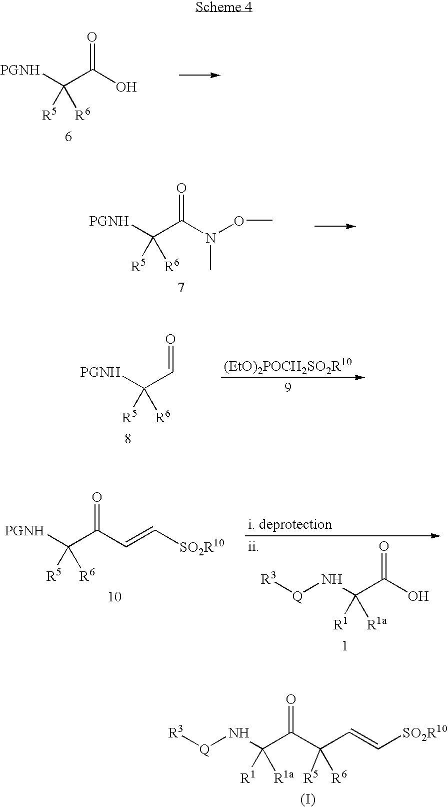

Reaction of an N-protected amino acid of formula 6 with N,O-dimethylhydroxylamine hydrochloride in the presence of 1 equivalent of triethylamine and N,N-dicyclohexylcarbodiimide forms the N,O-dimethylhydroxamate (Weinreb amide) 7, which is then reduced to the corresponding aldehyde 8 with a suitable reducing agent such as 0.5 equivalents of lithium aluminum hydride.

Condensation of 8 with a Wadsworth-Emmons reagent (EtO)₂POCH₂SO₂R¹ wherein R¹⁰ is as defined in the Summary of the Invention, affords the vinyl sulfone 10. Removal of the N-protecting group, followed by reaction of the resulting free amine with a compound of formula 1 under the reactions conditions described above then provides a compound of Formula (I).

Compounds of Formula (I) where Q is —HR— where R is haloalkyl, E and other groups are as defined in the Summary of the Invention can be prepared by proceeding as illustrated and described in Scheme 5 below:

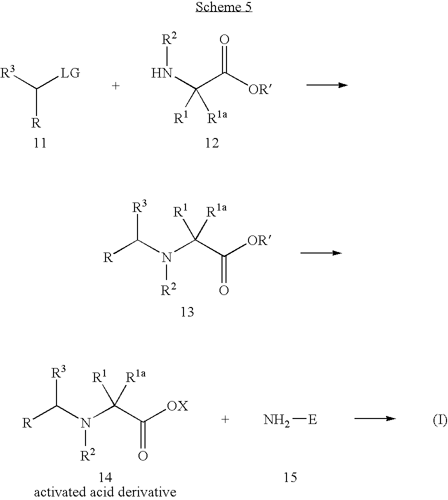

Reaction of a compound of formula 11 where LG is a suitable leaving group such as trifluoromethansulfonate, and the like, and R and R³ are as defined in Summary of the Invention with a compound of formula 12 where R¹, R^{1a}, and R² are as defined in the Summary of the Invention and R′ is hydrogen or a suitable carboxy protecting group such as alkyl, and the like, provides a compound of formula 13. The reaction is carried out in a suitable organic solvent, including but not limited to, diethyl ether, tetrahydrofuran, acetonitrile, benzene, toluene, xylene, and the like, or mixtures thereof and optionally in the presence of an organic or inorganic base. Preferably, the organic base is triethylamine, pyridine, N-methylmorpholine, collidine, diisopropylethylamine, and the like. Preferably, the inorganic base is cesium carbonate, sodium carbonate, sodium bicarbonate, and the like. The reaction is optionally carried out in the presence of a drying agent such as molecular sieves. Preferably, the reaction is carried out at room temperature.

Compounds of formula 11 can be prepared by methods well known in the art. For example, a compound of formula 11 where R⁶ is phenyl or 4-fluorophenyl, R is trifluoromethyl, and LG is trifluoromethylsulfonate can be readily prepared from commercially available 2,2,2-trifluoroacetophenone or 2,2,2,4′-tetrafluoroacetophenone respectively, by reducing the keto group to an alcoholic group with a suitable reducing agent such as sodium borohydride, lithium aluminum hydride, and the like. The solvent used depends on the type of reducing agent. For example, when sodium borohydride is used the reaction is carried out in an alcoholic organic solvent such as methanol, ethanol, and the like. When lithium aluminum hydride is used the reaction is carried out in an ethereal solvent such as tetrahydrofuran, and the like. Reaction of 2,2,2-trifluoro-1-phenylethanol or 2,2,2-trifluoro-1-(4-fluorophenyl)ethanol with triflic anhydride provides the desired compound. Optically enriched compound of formula 11 can be obtained by reduction of the corresponding halogenated acetophenone with a suitable reducing agent such as catecholborane or BH₃-DMS complex in the presence of a suitable catalyst such as (S) or (R)-CBS catalyst or (S) or (R)-α,α-diphenyl-2-pyrrolidine-methanol in the presence of BBN to provide chiral alcohol which is then converted to compound 11 as described above.

Compounds of formula 12 can be prepared by methods well known in the art. For example, compounds of formula 12 where R¹ is hydrogen and R^{1a} is -(alkylene)-SiR³²R³³R³⁴ where R³² is alkyl and R³³ and R³⁴ together with Si form a heterocycloalkyene ring containing 3 to 7 carbon atoms or R³² and R³³ are alkyl and R³⁴ is aryl can be prepared by following the procedure described in Smith, R. J. et al., Tetrahedron, 1997, Vol. 53, No. 40, pp 13695, the disclosure of which is incorporated herein by reference in its entirety. A compound of formula 12 where R¹ is hydrogen and R^{1a} is -(alkylene)-SiR³²R³³R³⁴ where R³² and R³³ are alkyl and R³⁴ is heterocycloalkylalkyl e.g., [(dimethyl)tetrahydropyan-4-ylmethylsilyl]alanine can be prepared by reacting dichloromethylsilane with buten-3-ylmagnesium bromide followed by tetrahydropyran-4-ylmethylmagnesium bromide to give 4-[(dimethyl)tetrahydropyan-4-ylmethylsilyl]buten-1-ene. Oxidation of 4-[(dimethyl)tetrahydropyan-4-ylmethylsilyl]buten-1-ene would provide 3-[(dimethyl)tetrahydropyan-4-ylmethylsilyl]propionic acid which can then be converted to [(dimethyl)tetrahydropyan-4-ylmethylsilyl]alanine under the conditions described in Smith, R. J. et. Al., Tetrahedron: Asymmetry, 2001, 157. A compound of formula 12 where R¹ is hydrogen and R^{1a} is 1,1-dialkylsilan-4-ylalkylene e.g., 1,1-dimethylsilinan-4-ylalanine can be prepared by reacting commercially available 1,1-dimethylsilinan-4-one with a Wittig reagent PH₃P═CH(CH₂)₂OH to provide 3-(1,1-dimethylsilinan-4-ylidene)propan-1-ol which upon reduction of the double bond under hydrogenation reaction conditions followed by oxidation would provide 3-(1,1-dimethylsilinan-4-ylidene)propionic acid which can be converted to 1,1-dimethylsilinan-4-ylalanine as described above. A compound of formula 12 where R³² is alkyl and R³³ and R³⁴ together with Si form a unsaturated heterocycloalkyene ring containing 3 to 7 carbon atoms e.g., (1-methyl-1,2,3,4-tetrahydrosilin-1-yl)alanine can be prepared by reacting 1,1-dichloro-1,2,3,4-tetrahydrosiline (Brook et. al., Can. J. Chem, 1970, 818) with methylmagnesium chloride followed by O-protected 3-propylmagnesium bromide to form O-protected 3-(1-methyl-1,2,3,4-tetrahydrosilin-1-yl)propanol. Removal of the oxygen protecting group followed by oxidation of the hydroxyl group would give 3-(1-methyl-1,2,3,4-tetrahydro-silin-1-yl)propionic acid which is converted to the desired compound as described above.

A compound of formula 12 where R³² is alkyl and R³³ and R³⁴ together with Si form a unsaturated heterocycloalkyene ring containing 3 to 7 carbon atoms where one of the carbon atoms is replaced by a heteroatom such as oxygen e.g., (4-methyl-[1,4]oxasilinan-4-yl)alanine can be prepared by treatment of (3-PGO-propyl)-ethoxy-methyl-(2-vinyloxyethyl)silane (via a procedure analogous to one described in Voronkov et al., J. Organomet. Chem., 1992, 289) with a suitable reducing agent such as lithium aluminum hydride to give (3-PGO-propyl)-methyl-(2-vinyloxyethyl)silane which upon treatment with chloroplatinic acid (see Voronkov et al., J. Organonmet. Chem., 1992, 289) would provide O-protected 3-(4-methyl-[1,4]oxasilinan-4-yl)propanol which can be converted to the desired compound as described above.

Removal of the carboxy protecting group from a compound of formula 13 where R′ is a protecting group provides a corresponding compound of formula 13 where R is hydrogen. The conditions used to remove the carboxy protecting group depend on the nature of the carboxy protecting group. For example, if R′ is alkyl, it is removed under basic hydrolysis reaction conditions utilizing aqueous base such as aqueous lithium hydroxide, sodium hydroxide, and the like in an alcoholic solvent such as methanol, ethanol, and the like.

Compound 13 (where R′ is H) is then converted to an activated acid derivative 14 (X is a leaving group) which upon reaction with an amine compound of formula 15 provides a compound of Formula (I). The activated acid derivative 14 can be prepared and then reacted with compound 15 in a stepwise manner or it can be generated in situ in the presence of compound 15. For example, if the activated acid 14 is an acid halide it is first prepared by reacting 13 (where R′ is H) with a halogenating agent such as thionyl chloride, oxalyl, chloride and the like and then reacted with compound 15. Alternatively, the activated acid derivative 14 is generated in situ by reacting compound 13 (where R′ is H) with 15 in the presence of a suitable coupling agent e.g., benzotriazole-1-yloxytrispyrrolidinophosphonium hexafluorophosphate (PyBOP®), O-benzotriazol-1-yl-N,N,N′,N′-tetramethyl-uronium hexafluorophosphate (HBTU), O-(7-azabenzotriazol-1-yl)-1,1,3,3-tetramethyl-uronium hexafluorophosphate (HATU), 1-(3-dimethylaminopropyl)-3-ethylcarbodiimide hydrochloride (EDC), 1,3-dicyclohexyl-carbodiimide (DCC), an the like, optionally in the presence of 1-hydroxybenzotriazole (HOBT), and in the presence of a base such as N,N-diisopropyl-ethylamine, triethylamine, N-methylmorpholine, and the like. Suitable reaction solvents are inert organic solvents such as halogenated organic solvents (e.g., methylene chloride, chloroform, and the like), acetonitrile, N,N-dimethylformamide, ethereal solvents such as tetrahydrofuran, dioxane, and the like.

Compounds of Formula (I) can also be prepared by methods disclosed in US and PCT applications publication Nos. US 2003/0092634A1, US 2003/0232863A1, US 2003/0134889, WO 02/098850, WO 03/024924, WO 00/55126, WO 03/037892, and WO 95/09838, and U.S. Pat. Nos. 6,506,733, 6,576,630, and 6,506,733 which are incorporated herein by reference in their entirety.

Additional Processes for Preparing Compounds of Formula (I):

A compound of the present invention can be prepared as a pharmaceutically acceptable acid addition salt by reacting the free base form of the compound with a pharmaceutically acceptable inorganic or organic acid. Alternatively, a pharmaceutically acceptable base addition salt of a compound of the present invention can be prepared by reacting the free acid form of the compound with a pharmaceutically acceptable inorganic or organic base; Inorganic and organic acids and bases suitable for the preparation of the pharmaceutically acceptable salts of compounds of the present invention are set forth in the definitions section of this application. Alternatively, the salt forms of the compounds of the present invention can be prepared using salts of the starting materials or intermediates.

The free acid or free base forms of the compounds of the present invention can be prepared from the corresponding base addition salt or acid addition salt form. For example, a compound of the present invention in an acid addition salt form can be converted to the corresponding free base by treating with a suitable base (e.g., ammonium hydroxide solution, sodium hydroxide, and the like). A compound of the present invention in a base addition salt form can be converted to the corresponding free acid by treating with a suitable acid (e.g., hydrochloric acid, etc).

The N-oxides of the compounds of the present invention can be prepared by methods known to those of ordinary skill in the art. For example, N-oxides can be prepared by treating an unoxidized form of the compound of the present invention with an oxidizing agent (e.g., trifluoroperacetic acid, permaleic acid, perbenzoic acid, peracetic acid, meta-chloroperoxy-benzoic acid, or the like) in a suitable inert organic solvent (e.g., a halogenated hydrocarbon such as dichloromethane) at approximately 0° C. Alternatively, the N-oxides of the compounds of the present invention can be prepared from the N-oxide of an appropriate starting material.

Compounds of the present invention in unoxidized form can be prepared from N-oxides of compounds of the present invention by treating with a reducing agent (e.g., sulfur, sulfur dioxide, triphenyl phosphine, lithium borohydride, sodium borohydride, phosphorus trichloride, tribromide, or the like) in an suitable inert organic solvent (e.g., acetonitrile, ethanol, aqueous dioxane, or the like) at 0 to 80° C.

Prodrug derivatives of the compounds of the present invention can be prepared by methods known to those of ordinary skill in the art (e.g., for further details see Saulnier et al. (1994), Bioorganic and Medicinal Chemistry Letters, Vol. 4, p. 1985). For example, appropriate prodrugs can be prepared by reacting a non-derivatized compound of the present invention with a suitable carbamylating agent (e.g., 1,1-acyloxyalkylcarbonochloridate, para-nitrophenyl carbonate, or the like).

Protected derivatives of the compounds of the present invention can be made by means known to those of ordinary skill in the art. A detailed description of the techniques applicable to the creation of protecting groups and their removal can be found in T. W. Greene, Protecting Groups in Organic Synthesis, 3^{rd} edition, John Wiley & Sons, Inc. 1999.

Compounds of the present invention may be conveniently prepared, or formed during the process of the invention, as solvates (e.g. hydrates). Hydrates of compounds of the present invention may be conveniently prepared by recrystallisation from an aqueous/organic solvent mixture, using organic solvents such as dioxin, tetrahydrofuran or methanol.

Compounds of the present invention can be prepared as their individual stereoisomers by reacting a racemic mixture of the compound with an optically active resolving agent to form a pair of diastereoisomeric compounds, separating the diastereomers and recovering the optically pure enantiomer. While resolution of enantiomers can be carried out using covalent diasteromeric derivatives of compounds of the present invention, dissociable complexes are preferred (e.g., crystalline diastereoisomeric salts). Diastereomers have distinct physical properties (e.g., melting points, boiling points, solubilities, reactivity, etc.) and can be readily separated by taking advantage of these dissimilarities. The diastereomers can be separated by chromatography or, preferably, by separation/resolution techniques based upon differences in solubility. The optically pure enantiomer is then recovered, along with the resolving agent, by any practical means that would not result in racemization. A more detailed description of the techniques applicable to the resolution of stereoisomers of compounds from their racemic mixture can be found in Sean Jacques Andre Collet, Samuel H. Wilen, Enantiomers, Racemates and Resolutions, John Wiley & Sons, Inc. (1981).

Preparation of Biological Agents

In practicing this invention several processes for the generation or purification of biological agents are used. Methods for preparing the biologics are well known in the art as discussed below.

Monoclonal antibodies are prepared using standard techniques, well known in the art, such as by the method of Kohler and Milstein, Nature 1975, 256:495, or a modification thereof, such as described by Buck et al. 1982, In Vitro 18:377. Typically, a mouse or rat is immunized with the MenB PS derivative conjugated to a protein carrier, boosted and the spleen (and optionally several large lymph nodes) removed and dissociated into single cells. If desired, the spleen cells may be screened (after removal of non-specifically adherent cells) by applying a cell suspension to a plate or well coated with the antigen. B-cells, expressing membrane-bound immunoglobulin specific for the antigen, will bind to the plate, and will not be rinsed away with the rest of the suspension. Resulting B-cells, or all dissociated spleen cells, are then induced to fuse with myeloma cells to form hybridomas. Representative murine myeloma lines for use in the hybridizations include those available from the American Type Culture Collection (ATCC).

Chimeric antibodies composed of human and non-human amino acid sequences may be formed from the mouse monoclonal antibody molecules to reduce their immunogenicity in humans (Winter et al. Nature 1991, 349:293; Lobuglio et al. Proc. Nat. Acad. Sci. USA 1989, 86:4220; Shaw et al. J. Immunol. 1987, 138:4534; and Brown et al. Cancer Res. 1987, 47:3577; Riechmann et al. Nature 1988, 332:323; Verhoeyen et al. Science 1988, 239:1534; and Jones et al. Nature 1986, 321:522; EP Publication No. 519,596, published Dec. 23, 1992; and U.K. Patent Publication No. GB 2,276,169, published Sep. 21, 1994).

Antibody molecule fragments, e.g., F(ab′).sub.2, FV, and sFv molecules, that are capable of exhibiting immunological binding properties of the parent monoclonal antibody molecule can be produced using known techniques. Inbar et al. Proc. Nat. Acad. Sci. USA 1972, 69:2659; Hochman et al. Biochem. 1976, 15:2706; Ehrlich et al. Biochem. 1980, 19:4091; Huston et al. Proc. Nat. Acad. Sci. USA 1988, 85(16):5879; and U.S. Pat. Nos. 5,091,513 and 5,132,405, to Huston et al.; and U.S. Pat. No. 4,946,778, to Ladner et al.

In the alternative, a phage-display system can be used to expand the monoclonal antibody molecule populations in vitro. Saiki, et al. Nature 1986, 324:163; Scharf et al. Science 1986, 233:1076; U.S. Pat. Nos. 4,683,195 and 4,683,202; Yang et al. J. Mol. Biol. 1995, 254:392; Barbas, III et al. Methods: Comp. Meth Enzymol. 1995, 8:94; Barbas, III et al. Proc. Natl. Acad. Sci. USA 1991, 88:7978.

The coding sequences for the heavy and light chain portions of the Fab molecules selected from the phage display library can be isolated or synthesized, and cloned into any suitable vector or replicon for expression. Any suitable expression system can be used, including, for example, bacterial, yeast, insect, amphibian and mammalian systems. Expression systems in bacteria include those described in Chang et al. Nature 1978, 275:615, Goeddel et al. Nature 1979, 281:544, Goeddel et al. Nucleic Acids Res. 1980, 8:4057, European Application No. EP 36,776, U.S. Pat. No. 4,551,433, deBoer et al. Proc. Natl. Acad. Sci. USA 1983, 80:21-25, and Siebenlist et al. Cell 1980, 20:269.

Expression systems in yeast include those described in Hinnen et al. Proc. Natl. Acad. Sci. USA 1978, 75:1929, Ito et al. J. Bacteriol. 1983, 153:163, Kurtz et al. Mol. Cell. Biol. 1986, 6:142, Kunze et al. J. Basic Microbiol. 1985, 25:141, Gleeson et al. J. Gen. Microbiol. 1986, 132:3459, Roggenkamp et al. Mol. Gen. Genet. 1986, 202:302, Das et al. J. Bacteriol. 1984, 158:1165, De Louvencourt et al. J. Bacteriol. 1983, 154:737, Van den Berg et al. Bio/Technology 1990, 8:135, Kunze et al. J. Basic Microbiol. 1985, 25:141, Cregg et al. Mol. Cell. Biol. 1985, 5:3376, U.S. Pat. Nos. 4,837,148 and 4,929,555, Beach et al. Nature 1981, 300:706, Davidow et al. Curr. Genet. 1985, 10:380, Gaillardin et al. Curr. Genet. 1985, 10:49, Ballance et al. Biochem. Biophys. Res. Commun. 1983, 112:284-289, Tilburn et al. Gene 1983, 26:205-221, Yelton et al. Proc. Natl. Acad. Sci. USA 1984, 81:1470-1474, Kelly et al. EMBO J. 1985, 4:475479; European Application No. EP 244,234, and International Publication No. WO 91/00357.

Expression of heterologous genes in insects can be accomplished as described in U.S. Pat. No. 4,745,051, European Application Nos. EP 127,839 and EP 155,476, Vlak et al. J. Gen. Virol. 1988, 69:765-776, Miller et al. Ann. Rev. Microbiol. 1988, 42:177, Carbonell et al. Gene 1988, 73:409, Maeda et al. Nature 1985, 315:592-594, Lebacq-Verheyden et al. Mol. Cell. Biol. 1988, 8:3129, Smith et al. Proc. Natl. Acad. Sci. USA 1985, 82:8404, Miyajima et al. Gene 1987, 58:273, and Martin et al. DNA 1988, 7:99. Numerous baculoviral strains and variants and corresponding permissive insect host cells from hosts are described in Luckow et al. Bio/Technology 1988, 6:47-55, Miller et al. GENERIC ENGINEERING, Setlow, J. K. et al. eds., Vol. 8, Plenum Publishing, pp. 1986, 277-279, and Maeda et al. Nature 1985, 315:592-594.

Mammalian expression can be accomplished as described in Dijkema et al. EMBO J. 1985, 4:761, Gorman et al. Proc. Natl. Acad. Sci. USA 1982, 79:6777, Boshart et al. Cell 1985, 41:521, and U.S. Pat. No. 4,399,216. Other features of mammalian expression can be facilitated as described in Ham et al. Meth. Enz. 1979, 58:44, Barnes et al. Anal. Biochem. 1980, 102:255, U.S. Pat. Nos. 4,767,704, 4,657,866, 4,927,762, 4,560,655 and Reissued U.S. Pat. No. RE 30,985, and in International Publication Nos. WO 90/103430, WO 87/00195.

The production of recombinant adenoviral vectors are described in U.S. Pat. No. 6,485,958.

Botulinum toxin type A can be obtained by establishing and growing cultures of Clostridium botulinum in a fermenter and then harvesting and purifying the fermented mixture in accordance with known procedures.

Any of the above-described protein production methods can be used to provide the biologic that would benefit from the present invention.

### Utility

The compounds of the invention are selective inhibitors of cysteine proteases, in particular, cathepsin S, K, B, and/or F, and accordingly are useful for treating diseases in which cysteine protease activity contributes to the pathology and/or symptomatology of the disease. For example, the compounds of the invention are useful in treating autoimmune disorders, including, but not limited to, juvenile onset diabetes, psoriasis, multiple sclerosis, pemphigus vulgaris, Graves' disease, myasthenia gravis, systemic lupus erythemotasus, rheumatoid arthritis and Hashimoto's thyroiditis, allergic disorders, including, but not limited to, asthma, allogenic immune responses, including, but not limited to, organ transplants or tissue grafts and endometriosis.

Cathepsin S is also implicated in disorders involving excessive elastolysis, such as chronic obstructive pulmonary disease (e.g., emphysema), bronchiolitis, excessive airway elastolysis in asthma and bronchitis, pneumonities and cardiovascular disease such as plaque rupture and atheroma. Cathepsin S is implicated in fibril formation and, therefore, of Formula (I) are useful in the treatment of systemic amyloidosis.

### Testing

The cysteine protease inhibitory activity, in particular, the Cathepsin S inhibitory activities of the compounds of the invention can be determined by methods known to those of ordinary skill in the art. Suitable in vitro assays for measuring protease activity and the inhibition thereof by test compounds are known. Typically, the assay measures protease-induced hydrolysis of a peptide-based substrate. Details of assays for measuring protease inhibitory activity are set forth in Biological Examples 1-6, infra.

### Administration and Pharmaceutical Compositions

In general, a compound of the present invention will be administered in therapeutically effective amounts via any of the usual and acceptable modes known in the art, either singly or in combination with one or more therapeutic agents. A therapeutically effective amount may vary widely depending on the severity of the disease, the age and relative health of the subject, the potency of the compound used and other factors. For example, therapeutically effective amounts of a compound of compounds of the present invention may range from about 10 micrograms per kilogram body weight (μg/kg) per day to about 20 milligram per kilogram body weight (mg/kg) per day, typically from about 100 μg/kg/day to about 10 mg/kg/day. Therefore, a therapeutically effective amount for a 80 kg human patient may range from about 1 mg/day to about 1.6 g/day, typically from about 1 mg/day to about 100 mg/day. In general, one of ordinary skill in the art, acting in reliance upon personal knowledge and the disclosure of this application, will be able to ascertain a therapeutically effective amount of a compound of the present invention for treating a given disease.

The compounds of the present invention can be administered as pharmaceutical compositions by one of the following routes: oral, systemic (e.g., transdermal, intranasal or by suppository) or parenteral (e.g., intramuscular, intravenous or subcutaneous). Compositions can take the form of tablets, pills, capsules, semisolids, powders, sustained release formulations, solutions, suspensions, elixirs, aerosols, or any other appropriate composition and are comprised of, in general, a compound of the present invention in combination with at least one pharmaceutically acceptable excipient. Acceptable excipients are non-toxic, aid administration, and do not adversely affect the therapeutic benefit of the active ingredient. Such excipient may be any solid, liquid, semisolid or, in the case of an aerosol composition, gaseous excipient that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk, and the like. Liquid and semisolid excipients may be selected from water, ethanol, glycerol, propylene glycol and various oils, including those of petroleum, animal, vegetable or synthetic origin (e.g., peanut oil, soybean oil, mineral oil, sesame oil, and the like). Preferred liquid carriers, particularly for injectable solutions, include water, saline, aqueous dextrose and glycols.

The amount of a compound of the present invention in the composition may vary widely depending upon the type of formulation, size of a unit dosage, kind of excipients and other factors known to those of skill in the art of pharmaceutical sciences. In general, a composition of a compound of the present invention for treating a given disease will comprise from 0.01% w to 10% w, preferably 0.3% w to 1% w, of active ingredient with the remainder being the excipient or excipients. Preferably the pharmaceutical composition is administered in a single unit dosage form for continuous treatment or in a single unit dosage form ad libitum when relief of symptoms is specifically required. Representative pharmaceutical formulations containing a compound of the present invention are described in working example below.

### EXAMPLES

The following preparations and examples are given to enable those skilled in the art to more clearly understand and to practice the present invention. They should not be considered as limiting the scope of the invention, but merely as being illustrative and representative thereof.

### SYNTHETIC EXAMPLES

### Reference A

### Synthesis of (R)-2-amino-3-trimethylsilanylpropionic acid

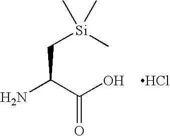 Step 1

To a stirred solution of the 3-(trimethylsilanyl)propionic acid (10 g, 68.5 mmol) in THF (100 ml) was added oxalyl chloride (8.9 ml, 102.7 mmol) and a drop of DMF at room temperature. After stirring for 2 h, the solvent and access of oxalyl chloride was removed under vacuum. The product 3-trimethylsilanylpropionyl chloride was used in the next step without further purification.

Step 2

To a stirred solution of(S)-4-benzyl-2-oxazolidinone (12.1 g, 68.5 mmol) in THF (100 ml) was added n-BuLi (1.6 M solution in hexane, 42.8 ml, 68.5 mmol) at −75° C. After stirring for 30 min, 3-trimethylsilanylpropionyl chloride was added and the reaction mixture was allowed to warm to room temperature and then quenched with saturated NH₄Cl, and extracted with ethyl acetate. The organic layer was washed with brine, dried with MgSO₄ and concentrated. The residue was purified by silica gel column chromatography to yield (S)-4-benzyl-3-[3-(trimethyl-silanyl)propionyl]oxazolidin-2-one (16.15 g).

Step 3

Sodium azide (21.45 g, 0.33 mol) was dissolved in of water-ethanol (300 ml, 1:1) and 2,4,6-triisopropylbenzenesulfonyl chloride (30.3 g, 0.1 mol) was added at room temperature. After stirred for 14 h, the reaction mixture was diluted with water and then extracted with ethyl ether. The organic layer was washed with brine, dried with MgSO₄, and the solvent was removed under vacuum. Methanol (50 ml) was added to the residue to give 2,4,6-triisopropylbenzenesulfonyl azide as a white crystalline solid (27.5 g).

Step 4

Into a solution of (S)-4-benzyl-3-[3-(trimethylsilanyl)propionyl]-oxazolidin-2-one (6.1 g, 20 mmol) in THF (50 ml) was added potassium bis(trimethylsilyl)amide (0.5 M solution in toluene, 44 ml, 22 mmol) at −65° C. After stirring for 2 h, 2,4,6-triisopropylbenzenesulfonyl azide (7.4 g, 24 mmol) in THF (50 ml) was added at −75° C. After stirring for 20 min, acetic acid (3 g) was added and the reaction mixture was allowed to warm to room temperature. 1N hydrochloric acid (11.2 ml) was added and the product was extracted with ethyl acetate. The organic layer was collected and washed with brine and dried with MgSO₄. The organics were removed to give a residue which was purified by silica gel column chromatography to yield (2R,4S)-4-benzyl-3-[3-(trimethylsilanyl)-2-azidopropionyl]oxazolidin-2-one (3.2 g).

Alternate Synthesis:

Tetrahydrofuran (120 ml) was cooled to −70° C. and then treated with potassium hexamethyldisilazide (0.5 M, 80 ml). A precooled solution of (S)-4-benzyl-3-[3-(trimethylsilanyl)propionyl]-oxazolidin-2-one (10.6 g) in THF (120 ml) was added at −66° C. over 15 min. A solution of 2,4,6-triisopropylbenzenesulfonyl azide (13.7 g) in tetrahydrofuran (120 ml) was added over 10 min. After 5 min, a solution of acetic acid (9 ml) in tetrahydrofuran (10 ml) was added and the reaction mixture warmed to 25° C. The reaction mixture was diluted with water, treated with sodium chloride and then extracted with ethyl acetate. The organic extracts were dried over magnesium sulfate and evaporated in vacuo. Chromatography of the residue on silica gel eluting with ethyl acetate—hexane mixtures gave (2R,4S)-4-benzyl-3-[3-(trimethylsilanyl)-2-azidopropionyl]oxazolidin-2-one as a colorless oil (9.06 g).

Step 5

(2R,4S)-4-Benzyl-3-[3-(trimethylsilanyl)-2-azidopropionyl]oxazolidin-2-one was dissolved in tetrahydrofuran (400 ml) and cooled to 0° C. and then treated with a solution of lithium hydroxide (1.09 g), water (140 ml), and 30% hydrogen peroxide (13.3 ml) over 35 min. After 75 min, a solution of sodium hydrogen sulfite (31 g) in water (140 ml) was added over 25 min. The tetrahydrofuran was removed by rotary evaporation and the product was isolated by extraction with ethyl acetate. Purification by silica gel chromatography eluting with ethyl acetate—hexane then gave (2R)-azido-3-trimethylsilypropionic acid (4.36 g).

Step 6

(2R)-Azido-3-trimethylsilypropionic acid (2.38 g) in methanol (120 ml) was treated with 10% Pd/C (130 mg) and hydrogenated at 48 psi for 1 h. The catalyst was removed by filtration through celite. Evaporation of the methanol then gave (R)-2-amino-3-trimethyl-silanylpropionic acid (1.50 g) as a white solid. LC-MS: 159.7(M−1); 161.7(M+1); 184(M+Na).

### Reference B

### Synthesis of (R)-2-amino-3-trimethylsilanylpropionic acid hydrobromide

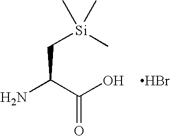 Step 1
(a) To a stirred solution of benzyloxycarbonyl-α-phosphonoglycine trimethyl ester (16.6 g, 50 mmol) in dichloromethane (50 ml) at room temperature was added DBU (8.4 g, 55 mmol). After stirring for 30 min, the reaction mixture was added to the following reaction mixture.
(b) To a stirred solution of oxalyl chloride (9.2 g, 72 mmol) in dichloromethane (150 ml) at −78° C. was added dimethyl sulfoxide (6.4 g, 82 mmol). After 15 min, a solution of trimethylsilylmethanol (5 g, 48 mmol) in dichloromethane (30 ml) was added over 10 min to the reaction mixture. After 30 min, triethylamine (17.94 g, 177.6 mmol) was added and after 30 min, the reaction mixture prepared in (a) was added at −78° C. After stirring for 15 min, the reaction mixture was allowed to warm up to room temperature and then quenched with 1N HCl. The organics were removed on roto-evaporator and the residue was extracted with ethyl ether. The organic layer was separated and washed with brine, dried with MgSO₄ and concentrated. The residue was purified by silica gel column chromatography to yield (Z)-2-benzyloxycarbonyl-amino-3-(trimethylsilanyl)acrylic acid methyl ester (5.1 g).
Step 2

To a solution of (Z)-2-benzyloxycarbonylamino-3-(trimethylsilanyl)-acrylic acid methyl ester (150 mg, 0.49 mmol) in ethyl acetate (3 ml) was added (+)-1,2-bis-(2S,5S)-2,5-diethylphospholanobenzene(cyclooctadiene) rhodium(I) trifluromethansulfonate (7 mg, 0.0098 mmol). The reaction mixture was stirred under hydrogen atomosphere at 5 psi for 2 h. Ethyl acetate was removed and the residue was purified by silica gel column chromatography to yield (R)-2-benzyloxycarbonylamino-3-(trimethylsilanyl)-propionic acid methyl ester (150 mg). e.e (>98%) was determined by analytical chiral column HPLC (Column: OD, solvent: 90% hexane, 10% isopropanol and 1 ml/min).

Step 3

To a stirred solution of (R)-2-benzyloxycarbonylamino-3-(trimethylsilanyl)propionic acid methyl ester (4.2 g, 13.6 mmol) in methanol (30 ml) was added 1N NaOH solution (20 ml) at room temperature. After stirring for 2 h, the reaction mixture was acidified with 1N HCl and extracted with ethyl acetate. The organic layer was washed with brine, dried with MgSO₄ and concentrated to give (R)-2-benzyloxycarbonylamino-3-(trimethylsilanyl)propionic acid (4 g).

Step 4

To a stirred flask contain (R)-2-benzyloxycarbonylamino-3-(trimethylsilanyl)propionic acid (4 g, 13.5 mmol) was added hydrogen bromide 33 wt % solution in acetic acid (10 ml). After stirring for 2 h, the access hydrogen bromide and acetic acid were removed under vacuum. Ethyl ether (40 ml) was added to the residue and after stirring for 30 min the solid was filtered, washed with ethyl ether, and dried to give (R)-2-amino-3-(trimethylsilanyl)propionic acid hydrogen bromide (3.2 g). H¹NMR (DMSO-d₆): δ 8.11 (3H, s), 3.82 (1H, t), 1.05 (2H, dd), 0.06 (9H, s). LC-MS: 160.1 (M−1); 161.8 (M+1).

### Reference C

### Synthesis of (S)-2-amino-1-benzoxazol-2-ylbutan-1-ol hydrochloride

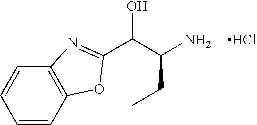 Step 1

To a solution of benzoxazole (28.6 g, 240 mmol) in toluene (150 ml), a 2 M solution of isopropyl-magnesium chloride in THF (120 ml, 240 mmol) was added during ca 20 min and at about −4° C. The red-brown mixture was stored at ca −4° C. and used as needed.

Step 2

To a solution of (S)-2-Boc-aminobutanol (50 g; 264 mmol) in dichloromethane (500 ml) and water (350 ml) were added at 20° C. TEMPO (0.01 eq), sodium bromide (1 eq) and sodium bicarbonate (3 eq). The reaction mixture was stirred at 0° C. and diluted bleach (1.3 eq, 450 ml) was added over 40 min. The reaction mixture was stirred for 30 min at 0° C. and then quenched with aq. thiosulfate. After decantation and extractions (dichloromethane), the organic phase was washed with brine, dried and concentrated in vacuo to dryness, giving (S)-2-(tert-butoxycarbonyl)-aminobutyraldehyde as a low-melting solid (38.1 g).

Step 3

A solution of (S)-2-(tert-butoxycarbonyl)amino-butyraldehyde (30 g, 160 mmol) in toluene (150 ml) was added over 30 min at −5° C. to a solution of Grignard reagent of benzoxazole (prepared as described in Step 1 above). The reaction mixture was stirred for 0.5 h at 0° C., then 2.5 h at RT. Quenching with 5% aq. acetic acid, washings with 5% aq. sodium carbonate, then brine and concentration to dryness gave crude (S)-2-(tert-butoxycarbonyl)-amino-1-benzoxazol-2-ylbutan-1-ol. The residue was diluted with toluene, and silica gel was added. The slurry was filtered. Elution by toluene removed the non-polar impurities. Then an 8/2 mixture of toluene and ethyl acetate desorbed the (S)-2-(tert-butoxycarbonyl)-amino-1-benzoxazol-2-ylbutan-1-ol.

Step 4

To a solution of (S)-2-(tert-butoxycarbonyl)amino-1-benzoxazol-2-yl-propan-1-ol (26.3 g, 86 mmol) in isopropanol (118 ml) at 20-25° C. was added trimethylchlorosilane (1.4 eq) and the solution was stirred for 5 h at 50° C. Concentration of the reaction mixture to 52 ml followed by addition of isopropyl ether (210 ml), filtration and drying under vacuum afforded (S)-2-amino-1-benzoxazol-2-ylbutan-1-ol hydrochloride salt as a grey solid (16.4 g mixture of diastereomers).

### Reference D

### Synthesis of 2(8)-(tert-butoxycarbonyl)amino-1-(oxazolo[4,5-b]pyridin-2-yl)butan-1-ol

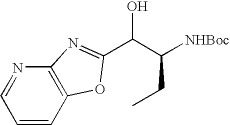 Step 1

A mixture of 2-amino-3-hydroxypyridine (11 g, 100 mmol), triethylorthoformate (80 ml) and p-toluenesulfonic acid (61 mg) was heated at 140° C. for 8 h. Excess triethylorthoformate was removed under vacuum and oxazolo[4,5-b]pyridine was crystallized from ethyl acetate (9 g).

Step 2

In a clean roundbottom flask equipped with stir bar was placed oxazolo[4,5-b]pyridine (600 mg, 5 mmol) in THF (30 ml) and the reaction mixture was cooled to 0° C. under N₂ atomosphere. Isopropylmagnesium chloride (2 M in THF, 2.5 ml, 5 mmol) was added. After stirring for 1 h at 0° C., (S)-2-(tert-butoxycarbonyl)aminobutyraldehyde (573 mg, 3 mmol) in THF (20 ml) was added. The ice bath was removed and the reaction mixture was allowed to warm to room temperature. After 2 h, the reaction mixture was quenched with saturated ammonium chloride solution and concentrated to dryness. The residue was extracted with EtOAc, then washed with brine, dried with anhyd. MgSO₄, filtered and concentrated. The crude product was purified by chromatography to yield the title compound (383 mg).

H¹NMR (DMSO-d₆): δ 8.42 (m, 1H), 8.18 (m, 1H), 7.3(m, 1H), 6.8-6.6 (dd, d, 1H, OH, diastereomer), 6.3-6.02 (d, d, 1H, NH, diastereomer), 4.82-4.5 (m,m, 1H, diastereomer), 1.8-1.3 (m, 2H), 1.2-1.05 (s,s, 9H, diastereomer), 0.89 (m, 3H). MS: 306.2 (M−1), 308.6 (M+1).

### Reference E

### Synthesis of (S)-2-amino-1-(3-phenyl-[1,2,4]oxadiazol-5-yl)butan-1-ol

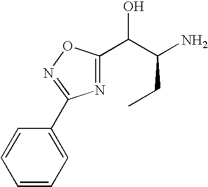

3-tert-Butoxycarbonylamino-2-hydroxypentanoic acid (500 mg, 2.14 mmol) was combined with EDC (600 mg, 3.14 mmol), HOBt (600 mg, 3.92 mmol), and N-hydroxy-benzamidine (292 mg, 2.14 mmol). Dichloromethane (10 ml) was added and then 4-methylmorpholine (1 ml) were added and the reaction mixture was stirred at ambient temperature for 16 h. After dilution with ethyl acetate (200 ml), the solution was washed with water (30 ml), saturated aqueous NaHCO₃ solution and brine, dried with MgSO₄ and evaporated under vacuum. The crude product was dissolved in pyridine (10 ml) and heated at 80° C. for 15 h. Pyridine was evaporated under vacuum and the residue was purified by flash chromatography on silica gel (eluent: ethyl acetate) to yield (S)-2-tert-butoxycarbonylamino-1-(3-phenyl-[1,2,4]oxadiazol-5-yl)-butan-1-ol (290 mg, 0.83 mmol). (S)-2-tert-butoxy-carbonylamino-1-(3-phenyl-[1,2,4]oxadiazol-5-yl)-butan-1-ol (145 mg, 0.41 mmol) was dissolved in CH₂Cl₂ (4 ml) and TFA (4 ml) was added. After stirring for 1 h, the reaction mixture was evaporated to dryness to yield (S)-2-amino-1-(3-phenyl-[1,2,4]oxadiazol-5-yl)-butan-1-ol.

Following the procedure described above but substituting N-hydroxy-benzamidine with N-hydroxypropamidine provided (S)-2-amino-1-(3-ethyl-[1,2,4]oxadiazol-5-yl)butan-1-ol.

### Reference F

### Synthesis of (S)-2-amino-1-(2-methoxymethyl-[1,3,4]oxadiazol-5-yl)butan-1-ol

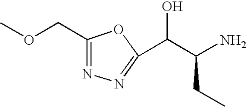 Step 1

(S)-(+)-2-amino-1-butanol (50 g, 561 mmol) in a mixture of water and dioxane (200 ml of water and 200 ml dioxane) was cooled to 0° C. and mixed with NaOH (26.9 g, 673 mmol) and di-tert-butyl-dicarbonate (146.96 g, 673 mmol). After the addition, the reaction was allowed to warm to room temperature and stirred for 2 h. After removing the dioxane, the residue was extracted with EtOAc, then washed with brine and dried with anhydrous MgSO₄, filtered and concentrated. Without further purification, the crude (S)-2-Boc-amino-1-butanol (120 g) was used for next step reaction.

Step 2

A solution of oxalyl chloride (40.39 g, 265 mmol) in CH₂Cl₂ (700 ml) was stirred and cooled to −60° C. Dimethylsulfoxide (51.7 g, 663 mmol) in CH₂Cl₂ (100 ml) was added dropwise. After 10 min, a solution of (S)-2-Boc-amino-1-butanol (50 g, 265 mmol) in CH₂Cl₂ (100 ml) was added dropwise at −70° C. The reaction mixture was allowed to warm to −40° C. for 10 min and then cooled to −70° C. again. A solution of triethylamine (74.9 g, 742 mmol) in CH₂Cl₂ (100 ml) was added and the reaction mixture was allowed to warm to room temperature over 2 h. Saturated sodium dihydrogen phosphate (100 ml) was added, and then the organic layer was washed with brine and dried over MgSO₄. The solvent was removed to yield (S)-2-Boc-amino-butyraldehyde(1-formylpropyl)carbamic acid tert-butyl ester (45 g).

Step 3

A mixture of methyl methoxyacetate (52 g, 500 mmol), hydrazine hydrate (30 ml) was heated to reflux for 8 h. Excess hydrazine and water were removed under vacuum. The residue was extracted with n-butanol, dried with Na₂SO₄. Excess n-butanol was removed to yield hydrazide (45 g).

Step 4

A mixture of above hydrazide (45 g), triethylorthoformate (146 ml) and p-toluene-sulfonic acid (61 mg) was heated at 140° C. for 8 h. Excess triethylorthoformate was removed under vacuum. The product was purified by silica gel column chromatography to yield 2-methoxymethyl-[1,3,4]-oxadiazole (4.6 g).

Step 5

To a stirred solution of 2-methoxymethyl-[1,3,4]-oxadiazole (4.6 g, 40 mmol) in THF (100 ml) was added n-BuLi (1.6 M solution in 25.2 ml of hexane) dropwise under N₂ at −78° C. After 1 h, MgBr.Et₂O (10.4 g, 40.3 mmol) was added and the reaction mixture was allowed to warm to −45° C. for 1 h before being treated with (S)-2-Boc-aminobutyraldehyde (5.28 g, 28.25 mmol) in THF (20 ml). The reaction mixture was stirred for 1 h, quenched with saturated NH₄Cl, and extracted with ethyl acetate. The organic layer was washed with brine, dried with MgSO₄ and concentrated. The residue was purified by silica gel column chromatography to yield (S)-2-Boc-amino-1-(5-methoxymethyl-[1,3,4]-oxadiazol-2-yl)-1-butanol (500 mg).

Step 6

2-Boc-amino-1-(5-methoxymethyl-[1,3,4]-oxadiazol-2-yl)-1-butanol (500 mg, 1.66 mmol), and CH₂Cl₂ (5 ml) were mixed and TFA (0.5 ml) was added at room temperature. After stirring for 1 h, the solvent and excess TFA were removed under vacuum to produce (S)-2-amino-1-(5-methoxymethyl-[1,3,4]oxadiazol-2-yl)-butan-1-ol TFA salt (340 mg).

### Reference G

### Synthesis of (S)-2-amino-1-(2-phenyl-[1,3,4]oxadiazol-5-yl)butan-1-ol

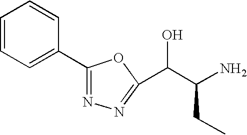 Step 1

A mixture of the benzoic hydrazide (22.5 g, 165 mmol), triethylorthoformate (150 ml) and p-toluenesulfonic acid (300 mg) was heated at 120° C. for 12 h. Excess triethylortho-formate was removed under vacuum and the residue was purified by silica gel column chromatography to produce 2-phenyl-[1,3,4]-oxadiazole (14.5 g).

Step 2

To a stirred solution of the 2-phenyl-[1,3,4]oxadiazole (10 g, 68.5 mmol) in THF (100 ml) was added n-BuLi (1.6 M solution in 42.8 ml of hexane) dropwise under N₂ at −78° C. After 1 h, MgBr.Et₂O (17.69 g, 68.5 mmol) was added and the reaction mixture was allowed to warm to −45° C. for 1 h before being treated with (S)-2-Boc-aminobutyraaldehyde (7.8 g, 41 mmol) in THF (20 ml). The reaction mixture was stirred for 1 h, quenched with saturated NH₄Cl, and extracted with ethyl acetate. The organic layer was washed with brine, dried with MgSO₄ and concentrated. The residue was purified by silica gel column chromatography to yield 2-((S)-2-Boc-amino-1-hydroxybutyl)-5-phenyl-[1,3,4]-oxadiazole (9.7 g).

Step 3

2-((S)-2-Boc-amino-1-hydroxybutyl)-5-phenyl-[1,3,4]-oxadiazole (505 mg, 1.5 mmol) and CH₂Cl₂ (5 ml) were mixed and TFA (1 ml) was added at room temperature. After stirring for 1 h, the solvent and excess TFA were removed under vacuum to produce (S)-2-amino-1-(5-phenyl-[1,3,4]oxadiazol-2-yl)-1-butanol TFA salt (530 mg).

### Reference H

### Synthesis of (S)-2-amino-1-oxazolo[4,5-b]pyridin-2-ylbutan-1-ol

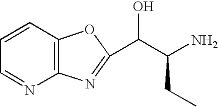 Step 1

A mixture of 2-amino-3-hydroxypyridine (25 g, 227 mmol), triethylorthoformate (75 ml) and p-toluenesulfonic acid (61 mg) was heated at 140° C. for 8 h. Excess triethylortho-formate was removed under vacuum. The product was crystallized from ethyl acetate to yield oxazolo[4,5-b]pyridine (22.5 g).

Step 2

To a stirred solution of the oxazolo[4,5-b]pyridine (12 g, 100 mmol) in THF (300 ml) was added n-BuLi (1.6 M solution in 62.5 ml of hexane) drop wise under N₂ at −78° C. After 1 h, MgBr.Et₂O (25.8 g, 100 mmol) was added and the reaction mixture was allowed to warm to −45° C. for 1 h before being treated with (S)-2-Boc-amino-butyraldehyde (11.46 g, 60 mmol) in THF (50 ml). The reaction mixture was stirred for 1 h, quenched with saturated NH₄Cl, and extracted with ethyl acetate. The organic layer was washed with brine, dried with MgSO₄ and concentrated. The residue was purified by silica gel column chromatography to yield (S)-2-Boc-amino-1-(oxazolo[4,5-b]pyridin-2-yl)-1-butanol (14.1 g).

Step 3

(S)-2-Boc-amino-1-(oxazolo[4,5-b]pyridin-2-yl)-1-butanol (311 mg, 1 mmol) and CH₂Cl₂ (5 mL) were mixed and TFA (1 mL) was added at room temperature. After stirring for 1 h, the solvent and excess TFA were removed under vacuum to provide (S)-2-amino-1-oxazolo[4,5-b]pyridin-2-yl-butan-1-ol TFA salt (355 mg).

### Reference I

### Synthesis of (S)-2-Boc-amino-1-(2-ethyl-[1,3,4]oxadiazol-5-yl)-1-butanol

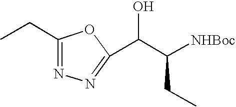 Step 1

A mixture of the formic hydrazide (60 g, 1 mole), triethylorthopropionate (176.26 g, 1 mole) and p-toluenesulfonic acid (250 mg) was heated at 120° C. for 12 hours. The ethanol was removed under vacuum and the residue was distilled under vacuum to yield ethyl-[1,3,4]-oxadiazole (24 g).

Step 2

To a stirred solution of the ethyl-[1,3,4]-oxadiazole (4.66 g, 48 mmol) in THF (50 ml) was added n-BuLi (1.6M solution in 30 ml of hexane) drop-wise under N₂ at −78° C. After 1 hour, MgBr.Et₂O (12.38 g, 48 mmol) was added and the reaction mixture was allowed to warm to −45° C. for 1 hour before being treated with (S)-2-Boc-aminobutyraldehyde (3.2 g, 24 mmol) in THF (20 ml). The reaction mixture was stirred for 1 hour, quenched with saturated NH₄Cl, and extracted with ethyl acetate. The organic layer was washed with brine, dried with MgSO₄ and concentrated. The residue was purified by silica gel column chromatography to yield the title compound (2.13 g). ¹NMR (DMSO-δ): 6.65-6.52 (1H, d, d, J=9.2 Hz, J=9.2 Hz, NH, diastereomer), 6.14, 5.95 (1H, d, d, J=5.6 Hz, J=5.6 Hz, OH, diastereomer), 4.758-4.467 (1H, m, diastereomer), 3.7-3.55 (1H, m), 2.8 (2H, q), 1.33(12H, t), 1.25-1.21 (2H, m), 0.82 (3H, m). MS: 284.1 (M−1), 286 (M+1), 308 (M+Na).

### Reference J

### Synthesis of 4-amino-4-cyano-1-ethylpiperidine

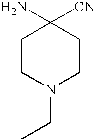

A mixture of 1-ethyl-4-piperidone (13.2 ml, 100 mmol), ammonium chloride (21.4 g, 400 mmol), sodium cyanide (19.6 g, 400 (mmol) and water (550 ml) was stirred at room temperature for 48 h. The pH of the reaction mixture was adjusted to 10.1 and the product was extracted with ethyl acetate. The organic extracts were washed with brine and dried over magnesium sulfate. Rotary evaporation of the solvent gave a mixture of 4-amino-4-cyano-1-ethyl piperazine and 4-hydroxy-4-cyano-1-ethyl piperazine (7.67 g). This mixture of products was treated with 7 M ammonia in methanol (20 ml) and allowed to stand at room temperature for 24 h. The methanol and excess ammonia were removed in vacuo and the residue was cooled to give 4-amino-4-cyano-1-ethylpiperidine as a crystalline solid (7.762 g).

### Reference K

### Synthesis of trifluoromethanesulfonic acid 2,2,2-trifluoro-1-(4-fluorophenyl)ethyl ester

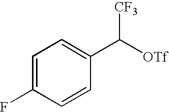 Step 1

To a stirred solution of 2,2,2,4′-tetrafluoroacetophen″one (10 g, 52.1 mmol) in methanol (50 mL) was added NaBH₄ (0.98 g, 26.5 mmol) at 0° C. After stirring at 25° C. for 2 h, the reaction mixture was quenched by adding 1N HCl (100 mL) and then extracted with ethyl ether. The ether extract was washed with brine, dried with MgSO₄, and concentrated to give 2,2,2-trifluoro-1-(4-fluorophenyl)ethanol (11.32 g) which was used in next step without further purification.

Step 2

NaH (640 mg, 16 mmol, 60% in mineral oil) was washed twice with hexane (20 mL) and then suspended in dried diethyl ether (20 mL). A solution of 2,2,2-trifluoro-1-(4-fluoro-phenyl)ethanol (1.94 g, 10 mmol) in diethyl ether (10 mL) was added at 0° C. After stirring for 2 h at room temperature, a solution of trifluoromethanesulfonyl chloride (1.68 g, 10 mmol) in diethyl ether (10 mL) was added. After 2 h, the reaction mixture was quenched by adding a solution of NaHCO₃ and the product was extracted with diethyl ether. The extracts were washed with brine and dried, and the solvent was removed to yield trifluoromethanesulfonic acid 2,2,2-trifluoro-1-(4-fluorophenyl)ethyl ester (3.3 g).

Proceeding as described in Example K above, trifluoromethanesulfonic acid 2,2,2-trifluoro-1-phenylethyl ester was prepared.

### Reference L

### Synthesis of 2,2,2-trifluoro-[(R)-(4-fluorophenyl)ethanol

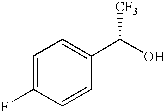

To a −78° C. toluene (25 mL)/dichloromethane (25 mL) solution of 2,2,2,4′-tetrafluoroacetophenone (2.5 g, 13.01 mmol) and 1M S-CBS catalyst (1.3 mL, 1.3 mmol) was added freshly distilled catecholborane (1.66 mL, 15.62 mmol). The reaction mixture was maintained at −78° C. for 16 h at which time 4N HCl (5 mL in dioxane) was added and the reaction mixture was allowed to warm to room temperature. The reaction mixture was diluted with ethyl acetate and washed with a saturated brine solution. The organic layer was dried over magnesium sulfate, filtered and concentrated to provide a solid. The solid was suspended in hexanes and filtered off. The hexanes filtrate containing the desired product was concentrated and the residue subjected to flash chromatography (10 hexanes: 1 ethylacetate) to provide the title compound as colorless oil (2.2 g, 87% yield). The ratio of enantiomers was determined to be 95:5 by chiral HPLC (Chiralcel OD column, 95 hexanes: 5 isopropanol mobile phase. Ret. time for major product was 6.757 min. Ret. time for minor isomer was 8.274 min.

### Reference M

### Synthesis of 1-aminocyclopropanecarbonitrile hydrochloride

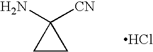 Step 1

A mixture of benzophenone imine (25 g, 0.138 mol, Aldrich) and aminoacetonitrile hydrochloride (25 g, 0.270 mol, Lancaster) in dichloromethane (1000 mL) was stirred in a 2L Erlenmeyer flask under nitrogen at room temperature for 5 days. The reaction mixture was filtered to remove the precipitated ammonium chloride and the filtrate was evaporated to dryness in vacuo. The resulting residue was dissolved in ether (400 mL) washed with water (200 mL) and brine. After drying over magnesium sulfate the solution was evaporated to give (benzhydrylideneamino)acetonitrile (47.89 g).

Step 2

A solution of sodium hydroxide (91 g, 2.275 mol) in water (91 mL) in a 2L flask was cooled on ice under nitrogen and then treated with benzyl triethyl ammonium chloride (2.0 g, 0.0088 mol, Aldrich) and (benzhydrylideneamino)acetonitrile (47.89 g) in toluene (100 mL). 1,2-Dibromoethane (23 mL, 122.4 mmol, Aldrich) was then added dropwise over 25 min, to the reaction mixture with mechanical stirring and cooling to maintain the internal temperature near +10° C. The reaction mixture was then stirred vigorously for 24 h at room temperature and then poured into ice water and extracted with toluene. The combined extracts were washed with brine and then treated with MgSO₄ and Norite. After filtering, toluene was removed by rotary evaporation to give an oil (67 g). The residue was dissolved in boiling hexane (400 mL), treated with Norite and filtered hot and allowed to cool. A dark oil separated and which was removed by pipet (˜2 mL). Scratching induced crystallization in the remaining solution which was cooled on ice for 2 h. Light yellow crystals were collected by filtration and washed with cold hexane to give 1-(benzhydrylideneamino)cyclopropane-carbonitrile (30.56 g).

Step 3

A mixture of 1-(benzhydrylideneamino)cyclopropanecarbonitrile (30.56 g, 0.124 mol) in concentrated HCl (12 mL) in water (100 mL) and ether (100 mL) was stirred at room temperature for 15 h. The ether layer was discarded and the aqueous layer was washed with ether. The aqueous layer was then freeze dried to give the title compound as a tan powder (13.51 g).

### Example 1

### Synthesis of 1-(R)-morpholine-4-carboxylic acid [1-(4-cyano-1-ethylpiperidin-4-ylcarbamoyl)-2-(trimethylsilanyl)ethyl]amide

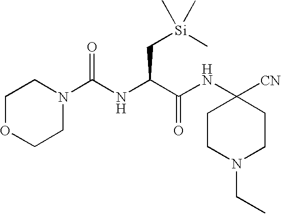 Step 1

A mixture of (R)-2-amino-3-trimethylsilanylpropionic acid (0.320 g, 2 mmol) and N-methyl-N-trimethylsilyltrifluoroacetamide (MSTFA) (1.85 g, 13 mmol) was heated at 70° C. for 1 h. The reaction mixture was cooled and the excess MSTFA was removed in vacuo. Morpholinocarbonyl chloride (0.70 ml, 6 mmol) was added to the reaction mixture which was heated at 70° C. for 45 min and then cooled. Water and ice (25 ml) was added to the reaction mixture which was stirred until the evolution of carbon dioxide ceased. The solution was extracted with ethyl acetate to give 2-(R)-[(morpholine-4-carbonyl)amino]-3-(trimethyl-silanyl)propionic acid (0.529 g) which was used in the following step without further purification.

Step 2

To a solution of 2-(R)-[(morpholine-4-carbonyl)amino]-3-(trimethylsilanyl)propionic acid (140 mg, 0.51 mmol) in DMF (2 ml) was added 4-amino-4-cyano-1-ethylpiperidine hydrochloride salt (99 mg, 0.52 mmol), HATU (296 mg, 0.78 mmol) and diisopropylethylamine (198 mg, 1.53 mmol) at room temperature. After 2 h, the reaction mixture was extracted with ethyl acetate, washed with brine, and dried. After removing the solvent, the residue was purified by silica gel column chromatography to yield the title compound (87 mg). H¹NMR (DMSO-d₆): δ 8.24 (1H, s), 6.5 (1H, d, J=8.8 Hz), 4.18 (1H, m), 3.6-3.48 (4H, m), 3.35-3.2 (4H, m), 2.75-2.55 (2H, m), 2.32 (2H, q, J=7.2 Hz), 2.3-2.1 (4H, m), 1.9-1.7 (2H, m), 0.98 (3H, t, J=7.2 Hz), 1.1-0.8 (2H, m), 0.009 (9H, s). MS: 408.4(M−1), 410.3(M+1), 432.1 (M+Na).

Proceeding as described in Example 1 above but substituting 4-amino-4-cyano-1-ethylpiperidine hydrochloride salt with 1-aminocyclopropanecarbonitrile provided 1-(R)-morpholine-4-carboxylic acid [1-(1-cyanocyclopropylcarbamoyl)-2-(trimethylsilanyl)-ethyl]amide.

H¹NMR (DMSO-d₆): δ 8.32 (1H, s), 8.04 (1H, s), 4.2 (1H, dd, J=7.2 Hz, J=14.4 Hz), 3.64 (4H, t, J=4.8 Hz), 3.31 (4H, m), 1.65-1.45 (2H, m), 1.25-1.15 (3H, m), 0.95-0.85 (1H, m), 0.008 (9H, s). MS: 337.3(M−1), 339(M+1), 361.1(M+Na).

Proceeding as described in Example 1 above but substituting 4-amino-4-cyano-1-ethyl-piperidine hydrochloride salt with 1-aminotetrahydrothiopyran-4-ylcarbonitrile provide 1-(R)-morpholine-4-carboxylic acid [1-(4-cyanotetrahydrothiopyran-4-ylcarbamoyl)-2-(trimethyl-silanyl)ethyl]amide. LC-MS: 397.1(M−1); 399.1(M+1); 421.3 (M+Na).

Proceeding as described in Example 1, Step 2 above, but substituting 4-amino-4-cyano-1-ethylpiperidine hydrochloride salt with 2-aminoacetonitrile and (R)-2-amino-3-trimethyl-silanylpropionic acid with (R)-2-benzyloxycarbonylamino-3-benzyldimethylsilanylpropionic acid (prepared as described in Reference B from dimethylbenzylsilylmethanol which was made from commercial available dimethylbenzylsilylmethane chloride as reference (J. Org. Chem., 1997, 62, 8962-8963) gave [2(R)-(benzyldimethylsilanyl)-1-(cyanomethylcarbamoyl)ethyl]-carbamic acid benzyl ester. ¹H-NMR(CDCl₃): 7.4-6.9(11H, m), 6.62(1H, NH), 5.1-5(2H, m), 4.14-4(3H, m), 2.1(2H, s), 1.63(1H, s), 1.14(1H, dd), 0.91(1H, dd), 0.01(6H, d). LC-MS: 408.3(M−1), 410.1(M+1), 432.2(M+Na).

Proceeding as described in Example 1, but substituting 4-amino-4-cyano-1-ethyl-piperidine hydrochloride salt with 1-aminocyclopropanecarbonitrile and morpholinocarbonyl chloride with 4-ethylpiperazin-1-ylcarbonyl chloride provided 1-(R)-4-ethylpiperazine 1-carboxylic acid [1-(1-cyanocyclopropylcarbamoyl)-2-(trimethylsilanyl)ethyl]amide. LC-MS:364.2(M−1), 66.1(M+1), 388.2(M+Na).

Proceeding as described in Example 1, Step 2 above, but substituting 4-amino-4-cyano-1-ethylpiperidine hydrochloride salt with 2-aminoacetonitrile and 2-(R)-[(morpholine-4-carbonyl)amino]-3-(trimethylsilanyl)propionic acid with (R)-2-benzyloxycarbonylamino-3-benzyldimethylsilanylpropionic acid provided [2(R)-(trimethylsilanyl)-1-(cyanomethyl-carbamoyl)ethyl]carbamic acid benzyl ester. LC-MS: 332.2(M−1), 333.9(M+1), 356.0(M+Na).

### Example 2

### Synthesis of 1-(R)-morpholine-4-carboxylic acid [1-(4-cyano-1,1-dioxohexahydro-1λ⁶-thiopyran-4-ylcarbamoyl)-2-(trimethylsilanyl)ethyl]amide

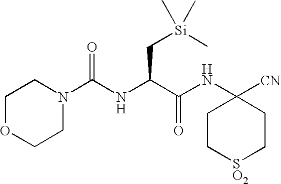

To a solution of crude 1-(R)-morpholine-4-carboxylic acid [1-(4-cyanotetrahydrothio-pyran-4-ylcarbamoyl)-2-(trimethylsilanyl)ethyl]amide (260 mg, 0.51 mmol) in MeOH (15 ml) was added oxone (469 mg, 0.76 mmol) in water (15 ml) at room temperature. After 2 h, MeOH was removed under vacuum and the residue was extracted with ethyl acetate. The ethyl acetate layer was washed with brine, dried, and concentrated. The residue was purified by silica gel column chromatography to yield the title compound (47 mg). H¹NMR (DMSO-d₆): δ 8.39 (1H, s), 6.5 (1H, d, J=7.6 Hz), 4.1 (1H, m), 3.49 (4H, t, J=4.4E1z), 3.4-3.1 (6H, m), 2.7-2.55 (2H, m), 2.5-2.4 (4H, m), 1.05-0.85(2H, m), 0.008 (9H, s). MS: 429.2(M−1), 431.1(M+1), 453.2 (M+Na).

### Example 3

### Synthesis of morpholine-4-carboxylic acid {1 (R)-[1(S)-(benzoxazol-2-ylhydroxymethyl)-butylcarbamoyl]-2-trimethylsilanylethyl}amide

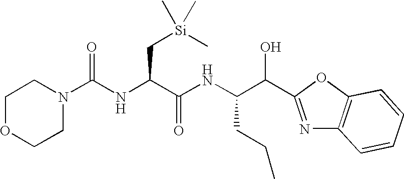

Into a solution of 2-(R)-2-[(morpholine-4-carbonyl)amino]-3-(trimethylsilanyl)-propionic acid (140 mg, 0.51 mmol) in CH₂Cl₂ (5 ml) was added 2-(S)-amino-1-benzoxazol-2-ylpentan-1-ol (121 mg, 0.55 mmol, prepared as described in Reference C), HOBt (95 mg, 0.62 mmol), EDC (148 mg, 0.77 mmol) and NMM (154 mg, 1.53 mmol) at room temperature. After 2 h, the reaction mixture was extracted with ethyl acetate and the organic layer was washed with brine, dried and concentrated. The residue was purified by silica gel column chromatography to yield the title compound (300 mg). LC-MS: 475.4(M−1); 477.5(M+1); 499.5 (M+Na).

### Example 4

### Synthesis of morpholine-4-carboxylic acid {1(R)-[1(S)-(benzoxazol-2-ylcarbonyl)-butylcarbamoyl]-2-trimethylsilanylethyl}amide

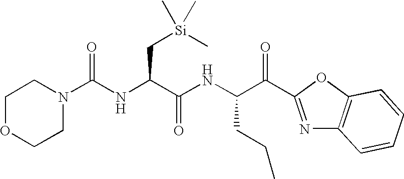

To a solution of crude morpholine-4-carboxylic acid {1(R)-[1(S)-(benzoxazol-2-ylhydroxymethyl)-butylcarbamoyl]-2-trimethylsilanylethyl}amide (300 mg) from Example 3 above, in CH₂Cl₂ (5 ml) was added Dess-Martin periodinane (324 mg, 0.76 mmol) at room temperature. After stirring for 1 h, saturated Na₂S₂O₃—NaHCO₃ (5 ml) were added. After a further 0.5 h, the reaction mixture was extracted with ethyl acetate, washed with brine, dried with MgSO₄ and concentrated. The residue was purified with silica gel column chromatography to yield the title compound (130 mg). H¹NMR (DMSO-d₆): δ 8.37 (1H, d, J=6 Hz), 8.0 (1H, d, J=7.6 Hz), 7.9 (1H, d, J=8.4 Hz), 7.65 (1H, d,t, J=1.6 Hz, J=7.2H2), 7.55 (1H, d, t, J=1.2 Hz, J=7.6 Hz), 6.42 (1H, d, J=8.8Hz), 6.21 (1H, m), 4.26 (1H, m), 3.51(4H, m), 3.35-3.2 (4H, m), 2.0-1.85 (1H, m), 1.8-1.65 (1H, m), 1.55-1.35(2H, m), 1-0.85 (5H, m), 0.008 (9H, s). MS: 473.3(M−1); 475.2(M+1); 497.3 (M+Na).

### Example 5

### Synthesis of morpholine-4-carboxylic acid {1(R)-[1(S)-(benzoxazol-2-ylcarbonyl)-propylcarbamoyl]-2-trimethylsilanylethyl}amide

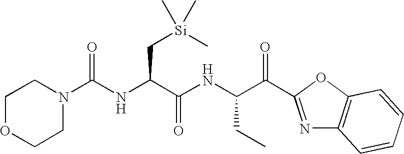 Step 1

To a solution of (Z/E)-2-benzyloxycarbonylamino-3-(trimethylsilanyl)acrylic acid methyl ester (Z/E=3:1, 43 g, 140.1 mmol) prepared as described in Reference B, Step 1 above, in ethyl acetate (100 ml) was added (+)-1,2-bis-(2S,5S)-2,5-diethylphospholanobenzene (cyclooctadiene) rhodium(I) trifluromethansulfonate (500 mg, 0.692 mmol) under nitrogen atmosphere. Hydrogen gas was introduced at 20 psi. After 2 h, ethyl acetate was removed by rotary evaporation to yield crude (R)-2-benzyloxycarbonylamino-3-(trimethylsilanyl)propionic acid methyl ester (43.8 g). Chiral HPLC analysis shows e.e>98%. (Column: OD, solvent: 90% hexane, 10% isopropanol and flow rate of 1 ml/min 20 psi).

Step 2

To a stirred solution of (R)-2-benzyloxycarbonylamino-3-(trimethylsilanyl)propionic acid methyl ester (43.8 g, 140 mmol) in methanol (300 ml) was added 1N NaOH solution (170 ml, 170 mmol) at 0° C. After completion of the addition, the reaction was allowed to warm to room temperature. After stirring for 2 h at room temperature, HPLC showed the reaction was completed. Methanol was removed by rotary evaporation and the residue was acidified with 1N HCl and extracted with ethyl acetate. The organic layer was washed with brine, dried with MgSO₄ and concentrated to give crude (R)-2-benzyloxycarbonylamino-3-(trimethylsilanyl)-propionic acid (42.7 g).

Step 3

To a stirred flask contain (R)-2-benzyloxycarbonylamino-3-(trimethylsilanyl)propionic acid (42.7 g, 145.2 mmol) was added hydrogen bromide in acetic acid (33 wt %, 90 ml). After stirring for 2 h, HPLC showed the starting material had been consumed. Ethyl ether (200 ml) was added and the reaction mixture was stirred for 30 min. The precipitated product was filtered, washed with ethyl ether and dried to yield (R)-2-amino-3-(trimethylsilanyl)propionic acid hydrogen bromide salt (22.5 g). The mother liquid was collected and the solvent was removed by rotary evaporation. The residue was stirred with mixture of a 1:1 mixture of ethyl ether and hexane (40 ml) to give additional 6 g of the product.

Step 4

A mixture of (R)-2-amino-3-(trimethylsilyl)propionic acid hydrobromide salt (1.439 g, 5.95 mmol) and N-methyl-N-trimethylsilyltrifluoroacetamide (MSTFA) (5.5 ml, 29.6 mmol) was heated at 69° C. for 55 min. The N-methyltrifluoroacetamide and excess MSTFA were removed by rotary evaporation and the resulting residue was treated with morpholinecarbonyl chloride (3.0 ml, 25 mmol) and heated again at 70° C. for 40 min. After cooling, water (30 ml) and a little ice were added to the reaction mixture which was stirred at room temperature until the evolution of CO₂ ceased (about 30 min). The reaction mixture was then extracted with ethyl acetate and the combined organic extracts were washed with brine, dried over magnesium sulfate, and concentrated to give morpholine-4-carboxylic acid (R)-2-(morpholin-4-ylcarbonyl)amino-3-(trimethylsilanyl)propionic acid (1.76 g) which was used in the next step without further purification.

Step 5

A mixture of morpholine-4-carboxylic acid (R)-2-(morpholin-4-ylcarbonyl)amino-3-(trimethylsilanyl)propionic acid (1.76 g), N-hydroxybenzotriazole (HOBt) (0.910 g, 5.95 mmol), (1-(3-dimethylaminopropyl)-3-ethylcarbodimide hydrochloride) (EDCI) (1.370 g, 7.14 mmol), and (R)-2-amino-1-benzoxazol-2-yl-propan-1-ol (1.472 g, 7.14 mmol) in methylene chloride (15 ml) was cooled on ice and treated with N-methylmorpholine (0.910 ml, 8.9 mmol). The reaction mixture was stirred at room temperature for 2 h and was then poured into a solution of water (50 ml), 1N HCl (15 ml), brine (50 ml) and ice. The product was extracted with ethyl acetate and the combined organic layers were washed with saturated NaHCO₃ and then brine. The extracts were dried over magnesium sulfate and evaporated to give morpholine-4-carboxylic acid {1(R)-[1(S)-(benzoxazol-2-ylhydroxymethyl)propylcarbamoyl]-2-trimethylsilanylethyl}amide (2.476 g, 5.36 mmol) as a semi-solid mixture of diasteromers.

Step 6

A solution of morpholine-4-carboxylic acid{1(R)-[1(S)-(benzoxazol-2-ylhydroxy-methyl)propylcarbamoyl]-2-trimethylsilanylethyl}amide (2.476 g) in methylene chloride (33 ml) was cooled on an ice/salt bath to −2° C. and treated with sodium bromide (0.612 g, 6 mmol), sodium bicarbonate (0.504 g, 6 mmol), and 2,2,6,6-tetramethyl-1-piperidinyloxy (TEMPO) (0.06 mmol). A solution of sodium hypochlorite (11 ml, 6%, 9 mmol) (Commercial laundry bleach was used as the oxidant and the quantity was calculated assuming the weight of sodium hypochlorite to be 6.5%) in water (23 ml) was then added dropwise to the reaction mixture with rapid stirring over 45 min while the internal reaction temperature was maintained near 0° C. After the reaction was complete (HPLC), the reaction was quenched by addition of 10% aqueous sodium thiosulfate (10 ml). The methylene chloride layer was separated and the aqueous layer was extracted with methylene chloride. The combined organic layers were washed with water, then brine and dried over magnesium sulfate. Evaporation of the solvent gave a tan colored foam (1.879 g) which was dissolved in i-propyl acetate (3 ml), diluted with tert-butylmethyl ether (8 ml) and cooled in the freezer overnight. Filtration of the solid gave the title compound (1.396 g).

Proceeding as described in Example 5 above, but substituting (R)-2-amino-1-benzoxazol-2-yl-propan-1-ol with (RS)-2-amino-1-benzoxazol-2-yl-propan-1-ol, followed by separation of the diastereomer provided morpholine-4-carboxylic acid {1(R)-[1(R)-(benzoxazol-2-ylcarbonyl)-propylcarbamoyl]-2-trimethylsilanylethyl}amide. ¹H-NMR(DMSO-d₆): 7.825(1H, d), 7.57(1H, d), 7.47(1H, dd), 7.39(1H, dd), 7.17(1H, d), 5.6-5.4(1H, m), 4.77(1H, d), 4.5-4.4(1H, m), 3.65-3.55(4H, m), 3.35-3.25(4H, m), 2.15-2(1H, m), 1.9-1.8(1H, m), 1.2-1.1(2H, m), 0.94(3H, t), 0.01-0(11H, m). LC-MS: 459.3(M−1), 461.3(M+1), 483.2(M+Na).

### Example 6

### Synthesis of 3′-cyanobiphenyl-3-carboxylic acid [1-RS-(1-cyanocyclopropylcarbamoyl)-2-(trimethylsilanyl)ethyl]amide

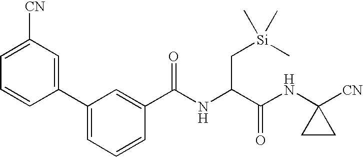 Step 1

A mixture of 3-iodobenzoic acid (21.73 g, 0.0876 mol), benzene (75 ml), 2 drops of dimethyl formamide, and thionyl chloride (10 ml, 0.137 mol) was heated at 82° C. for 2 h at which time a bubbler showed no further sulfur dioxide release. The solvent was removed at reduced pressure to give 3-iodobenzoyl chloride. In a separate flask a solution of diethylamino malonate hydrochloride (18.3 g, 0.086 mol) in methylene chloride (100 ml) was prepared and cooled to −18° C. N-Methylmorpholine (22 ml, 0.20 mol) was added followed by the 3-iodobenzoyl chloride prepared above at a rate which kept the reaction temperature below −7° C. The reaction mixture was allowed to warm to room temperature and then stirred for 3 h. The reaction mixture was poured into ice water and extracted with methylene chloride. The organic layers were washed with dilute HCl, aqueous sodium bicarbonate and brine. After drying over magnesium sulfate the solvent was removed and crystallization from tert-butylmethyl ether gave 2-(3-iodobenzoyl-amino)malonic acid diethyl ester (23.87 g).

Step 2

A mixture of 2-(3-iodobenzoylamino)malonic acid diethyl ester (16.08 g, 0.0397 mol), cesium carbonate (23.2 g, 1.8 equivalents), iodomethyltrimethylsilane (10.6 ml, 1.8 equivalents) and N-methylpyrrolidinone (50 ml) was heated at 71° C. for 6 h. The cooled reaction mixture was poured into ice water and extracted with ethyl acetate. The extracts were washed with brine, dried over magnesium sulfate and evaporated under reduced pressure. Flash chromatography on silica gel eluting with ethyl acetate/hexane followed by crystallization gave 2-(3-iodobenzoylamino)-2-trimethylsilanylmethylmalonic acid diethyl ester (8.82 g).

Step 3

A solution of 2-(3-iodobenzoylamino)-2-trimethylsilanylmethylmalonic acid diethyl ester (8.419 g, 0.0171 mol), lithium bromide (2.19 g, 0.025 mol), dimethylformamide (25 ml) and water (0.75 ml) was heated in a flask equipped with a bubbler to 150° C. for 4 h. After cooling to room temperature, the reaction mixture was poured into ice water and extracted with ethyl acetate. The extracts were dried and evaporated to give 2-(RS)-(3-iodobenzoyl-amino)-3-(trimethyl-silanyl)propionic acid ethyl ester (6.73 g).

Step 4

A mixture of 2-(RS)-(3-iodobenzoylamino)-3-(trimethylsilanyl)propionic acid ethyl ester (6.73 g, 0.016 mol), methanol (100 ml) and 1 N aqueous sodium hydroxide (40 ml) was stirred at room temperature for 1.5 h. The methanol was removed by evaporation under reduced pressure and the remaining aqueous solution was washed with ether, cooled on ice, and acidified to pH 2. The product precipitated from the aqueous layer and was collected by filtration to yield 2-(RS)-(3-iodobenzoylamino)-3-(trimethylsilanyl)propionic acid (6.25 g).

Step 5

A mixture of 2-(RS)-(3-iodobenzoylamino)-3-(trimethylsilanyl)propionic acid (4.88 g, 0.0125 mol), dimethyl formamide (25 ml), 1-amino-1-cyanocyclopropane hydrochloride (1.95 g, 0.016 mol), N-[(dimethylamino-1H-1,2,3-triazolo[4,5-b]pyridin-1-ylmethyl]-N-methyl-methaneaminium hexafluorophosphatre-N-oxide (HATU) (5.70 g, 1.2 equivalents) and N-methylmorpholine (4.13 ml) was stirred at room temperature for 4 h. The reaction mixture was diluted water and then extracted with ethyl acetate. The extracts were washed with dilute HCl, saturated sodium bicarbonate and brine. Drying and evaporation of the solvent gave a residue that was crystallized from t-butyl methyl ether to give N-[1-(RS)-(1-cyano-cyclopropylcarbamoyl)-2-(trimethylsilanyl)ethyl]-3-iodobenzamide (4.079 g).

Step 6

A mixture of N-[1-(RS)-(1-cyanocyclopropylcarbamoyl)-2-(trimethylsilanyl)ethyl]-3-iodo-benzamide (0.091 g, 0.0002 mol), toluene (2.5 ml), 2N sodium carbonate (0.20 ml,), ethanol (0.1 ml), 3-cyanophenyl boronic acid (0.030 g, 0.0002 mol) and tetrakis(triphenylphosphine)-palladium(0) (0.015 g) was heated at 105° C. for 14 h. The reaction mixture was cooled to room temperature, diluted with water and extracted with ethyl acetate. The extracts were washed with brine and dried over magnesium sulfate, Evaporation gave 0.106 g of crude product which was chromatographed on silica gel to give 3′-cyanobiphenyl-3-carboxylic acid [1-(RS)-(1-cyano-cyclopropylcarbamoyl)-2-(trimethyl-silanyl)ethyl]amide (0.047 g).

Proceeding as described above but substituting suitable boronic acids for 3-cyano-phenylboronic acid the following analogs were prepared:
3′-trifluoromethoxybiphenyl-3-carboxylic acid [1-(RS)-(1-cyanocyclopropyl-carbamoyl)-2-trimethylsilanylethyl]amide;
biphenyl-3-carboxylic acid [1-(RS)-(1-cyanocyclopropylcarbamoyl)-2-trimethyl-silanylethyl]amide;
2′,6′-dimethoxybiphenyl-3-carboxylic acid [1-(RS)-(1-cyanocyclopropylcarbamoyl)-2-trimethylsilanylethyl]amide;
4′-methylsulfonylbiphenyl-3-carboxylic acid [1-(RS)-(1-cyanocyclopropylcarbamoyl)-2-trimethylsilanylethyl]amide;
2′-chlorobiphenyl-3-carboxylic acid [1-(RS)-(1-cyanocyclopropylcarbamoyl)-2-trimethylsilanylethyl]amide;
2′-trifluoromethylbiphenyl-3-carboxylic acid [1-(RS)-(1-cyanocyclopropylcarbamoyl)-2-trimethylsilanylethyl]amide;
N-[1-(RS)-(1-cyanocyclopropylcarbamoyl)-2-trimethylsilanylethyl]-3-pyridin-3-ylbenzamide;
3′-methylbiphenyl-3-carboxylic acid [1-(RS)-(1-cyanocyclopropylcarbamoyl)-2-trimethylsilanylethyl]amide;
3′-hydroxymethylbiphenyl-3-carboxylic acid [1-(RS)-(1-cyanocyclopropylcarbamoyl)-2-trimethylsilanylethyl]amide;
4′-hydroxymethylbiphenyl-3-carboxylic acid [1-(RS)-(1-cyanocyclopropylcarbamoyl)-2-trimethylsilanylethyl]amide;
3′-methoxycarbonylbiphenyl-3-carboxylic acid [1-(RS)-(1-cyanocyclopropylcarbamoyl)-2-trimethylsilanylethyl]amide; and
4′-acetylbiphenyl-3-carboxylic acid [1-(RS)-(1-cyanocyclopropylcarbamoyl)-2-trimethylsilanylethyl]amide.

### Example 7

### Synthesis of 3′-methoxybiphenyl-3-carboxylic acid [1-(RS)-(4-cyano-1,1-dioxo-hexahydro-1λ⁶-thiopyran-4-ylcarbamoyl)-2-(trimethylsilanyl)ethyl]amide

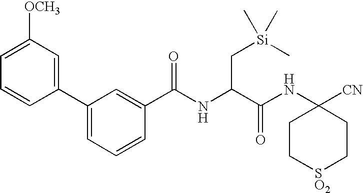 Step 1

Proceeding as described in Example 6, Step 5 above but substituting 1-amino-1-cyanocyclopropane hydrochloride with 4-amino-tetrahydro-thiopyran-4-carbonitrile provided N-[1-(RS)-(4-cyanotetrahydrothiopyran-4-ylcarbamoyl)-2-(trimethylsilanyl)ethyl]-3-iodobenzamide.

Step 2

A mixture of N-[1-(RS)-4-cyanotetrahydrothiopyran-4-ylcarbamoyl)-2-(trimethyl-silanyl)ethyl]-3-iodobenzamide (0.90 g, 0.177 mmol), 3-methoxyphenylboronic acid (0.031 g, 0.20 mmol), toluene (2.5 ml), ethanol (0.10 ml), aqueous sodium carbonate (2 N, 0.20 ml) and tetrakis triphenylphosphinepalladium(0) (0.010 g) was heated at 90° C. for 16 h and then cooled to room temperature, diluted with water and extracted with ethyl acetate. The extracts were washed with brine, dried over magnesium sulfate and evaporated to give a crude product which was chromatographed on silica gel and crystallized to give 3′-methoxybiphenyl-3-carboxylic acid [1-(RS)-(4-cyanotetrahydrothiopyran-4-ylcarbamoyl)-2-(trimethylsilanyl)-ethyl]amide (0.040 g). Rechromatography of impure fractions gave another 0.009 g of the product.

Step 3

A mixture of 3′-methoxybiphenyl-3-carboxylic acid [1-(RS)-(4-cyanotetrahydrothio-pyran-4-ylcarbamoyl)-2-(trimethylsilanyl)ethyl]amide (0.047 g, 0.095 mmol) in methanol (4 ml) was cooled on ice and treated with a solution of oxone (0.08 7 g, 1.5 equivalents) in water (1.0 ml). After 1 h of stirring at room temperature, additional oxone (0.070 g) in water (0.5 mL) was added. The reaction mixture was stirred at room temperature for 6 h and then diluted with water. The product was extracted with ethyl acetate and purified to give the title compound (0.006 g).

### Example 8

### Synthesis of 3′-methoxybiphenyl-3-carboxylic acid [1-(R)-(1-cyanocyclopropylcarbamoyl)-2-(trimethyl-silanyl)ethyl]amide

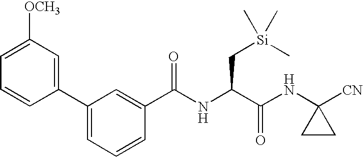 Step 1

2-(R)-Amino-3-(trimethylsilanyl)propionic acid (0.424 g, 0.0020 mol), in water (5 ml), and dioxane (10 ml) was cooled on an ice bath and treated with aqueous 2 N potassium hydroxide (3 ml). A solution of di-tert-butyl dicarbonate (0.545 g, 0.0025 mol) in dioxane (2 ml) was then added in portions and the reaction mixture was stirred at room temperature for 6 h. The reaction mixture was cooled on ice and then acidified with 1 N HCl to pH2.8 and extraction with ethyl acetate gave 2-(R)-tert-butoxycarbonylamino-3-(trimethylsilanyl)-propionic acid (0.588 g).

Step 2

A mixture of 2-(R)-tert-butoxycarbonylamino-3-(trimethylsilanyl)propionic acid (0.497 g, 0.0188 mol), dimethyl formamide (4 ml), HATU (0.80 g, 0.0021 mol), 1-amino-1-cyanocyclopropane hydrochloride (0.300 g, 0.0025 mol) and N-methylmorpholine (0.44 ml) was stirred at room temperature for 16 h. The reaction mixture was diluted with 0.5 N HCl and extracted with ethyl acetate. The extracts were washed with sodium bicarbonate then brine, dried over magnesium sulfate and evaporated. Flash chromatography gave [1-(R)-(1-cyano-cyclopropylcarbamoyl)-2-(trimethylsilanyl)ethyl]carbamic acid tert-butyl ester (0.323 g).

Step 3

A solution of [1-(R)-(1-cyanocyclopropylcarbamoyl)-2-(trimethylsilanyl)ethyl]-carbamic acid tert-butyl ester (0.160 g, 0.49 mmol) methane sulfonic acid (0.20 ml) and tetrahydrofuran (3 ml) was stirred at room temperature for 48 h. The reaction mixture was diluted with aqueous sodium bicarbonate and the product was extracted with ethyl acetate. Drying and evaporating gave [1-(R)-(1-cyanocyclopropylcarbamoyl)-2-(trimethylsilanyl)-ethyl]carbamic acid (0.090 g).

Step 4

A mixture of [1-(R)-(1-cyanocyclopropylcarbamoyl)-2-(trimethylsilanyl)ethyl]-carbamic acid (0.076 g, 0.337 mmol), methylene chloride (3.5 ml), 3-carboxyphenyl boronic acid (0.067 g, 0.405 mmol), HATU (0.282 g, 2.2 equivalents) and N-methyl morpholine (0.081 ml) was stirred at room temperature for 18 h. The reaction mixture was poured into dilute HCl and the product was extracted with ethyl acetate and the extracts were washed with aqueous sodium bicarbonate and brine. After drying the solvent was removed to give N-[1-(R)-(1-cyanocyclopropylcarbamoyl)-2-(trimethylsilanyl)ethyl]-3-boronic benzamide as a white powder (0.202 g).

Step 5

A mixture of N-[1-(R)-(1-cyanocyclopropylcarbamoyl)-2-(trimethylsilanyl)ethyl]-3-boronic benzamide (0.184 g, 0.493 mmol), 3-bromoanisole (0.075 ml, 0.596 mmol), triethylamine (0.034 ml, 2.46 mmol), Pd(dppf) (0.041 g, 0.1 equivalents) in acetonitrile (2 ml) was heated in a microwave apparatus at 130° C. for 10 min. The reaction mixture was diluted with ethyl acetate and washed with dilute HCl, aqueous sodium bicarbonate and brine. After drying over magnesium sulfate and removal of the solvent the residue was purified by flash chromatography to give 3′-methoxybiphenyl-3-carboxylic acid [1-(R)-(1-cyanocyclopropyl-carbamoyl)-2-(trimethyl-silanyl)ethyl]amide (0.023 g).

### Example 9

### Synthesis of 3-(benzyldimethylsilanyl)-N-(1-cyanocyclopropyl)-2(R)-(2,2,2-trifluoro-1-phenyl-ethylamino)propionamide

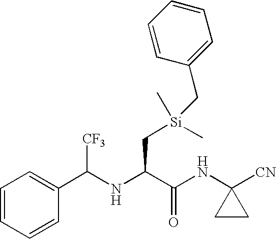 Step 1

Into 3-(benzyldimethylsilanyl)-2-(R)-benzyloxycarbonylaminopropionic acid methyl ester (1.93 g, 5 mmol) was added 30% of HBr in AcOH solution (5 ml) at room temperature. After stirred for 30 min, the reaction was diluted with toluene (50 ml) and then the solvent was removed by rotoevaporation. The residue was dissolved in ethyl acetate and washed with saturated NaHCO₃ water solution and brine, and dried over MgSO₄. After concentration obtained 2-(R)-amino-3-(benzyldimethylsilanyl)propionic acid methyl ester (1.96 g).

Step 2

Into a solution of 2-(R)-amino-3-(benzyldimethylsilanyl)propionic acid methyl ester (1.96 g) in dichloromethane (20 ml) was added trifluoroacetophenone (0.87 g, 5 mmol), DIPEA (2.59 g, 20 mmol) and 1 M of TiCl₄ solution in CH₂Cl₂ (5 ml, 5 mmol) at room temperature. After stirring for 4 h, additional 1 M solution of TiCl₄ in CH₂Cl₂ (3 ml) was added. After 12 h, NaBH₃CN (1.28 g, 20 mmol) in MeOH (20 ml) was added. After 2 h, the reaction was extracted with CH₂Cl₂ (150 ml) and washed with brine and dried over MgSO₄. After column chromatography obtained 3-(benzyldimethylsilanyl)-2(R)-(2,2,2-trifluoro-1-phenethylamino)propionic acid methyl ester (0.4 g).

Step 3

Into a solution of 3-(benzyldimethylsilanyl)-2(R)-(2,2,2-trifluoro-1-phenethylamino)-propionic acid methyl ester (0.4 g, 0.98 mmol) in a mixture of THF/MeOH (10 ml/5 ml) was added 1 M aqueous solution of LiOH (3 ml) at room temperature. After stirring for 2 h, the solvent was removed by rotoevaporation, the residue was diluted with pH 4 buffer and extracted with ethyl acetate (150 ml). After washing the organic layer with brine and drying over MgSO₄, the solvent was removed by rotoevaporation to give 3-(benzyldimethylsilanyl)-2(R)-(2,2,2-trifluoro-1-phenethylamino)propionic acid (395 mg).

Step 4

Into a solution of 3-(benzyldimethylsilanyl)-2(R)-(2,2,2-trifluoro-1-phenylethylamino)-propionic acid (395 mg, 1 mmol) in DMF (10 ml) was added HATU (380 mg, 1 mmol) DIPEA (258 mg, 2 mmol) and cyclopropylaminonitrile hydrochloride salt (119 mg, 1 mmol) at room temperature. After 2 h, the reaction mixture was extracted with ethyl acetate (150 ml), washed with brine and dried with MgSO₄. After removal the solvent, the crude was purified by column chromatography to give the title compound (229 mg)

HNMR (DMSO-d₆): 8.92, 8.86(1H, s, diastereomer), 7.6-7(10H, m), 4.4-4.2 (1H, m), 3.8(1H, s), 3.5-3.4, 3.1-2.9(1H, m, diastereomer), 2.65-2.5(2H, m), 2.35-2.1(2H, m), 1.5-1.4(2H, m), 1.1-0.85(2H, m), 0.126, 0.093, 0.039, −0.001(6H, d, diastereomer). LC-MS: 458.1(M−1), 460.2(M+1), 482.3(M+Na).

### Example 10

### Synthesis of morpholine-4-carboxylic acid [1-(RS)-(1-benzyloxymethyl-1-cyanopropylcarbamoyl)-2-trimethylsilanylethyl]amide

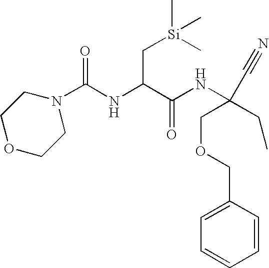 Step 1

A solution of commercially available benzoyloxyacetaldehyde (1 g, 6.66 mmol) in THF (10 ml) was added a 1 M solution of EtMgBr in THF (6.66 ml, 6.66 mmol) under N₂ atmosphere. The reaction mixture was stirred at room temperature for 2 h and then quenched with 5 ml of water and filtered through celite. The celite was washed with EtOAc and the filtrate was washed with brine and dried over MgSO₄. The organic layer was filtered and evaporated to dryness to give 1-benzyloxybutan-2-ol (1 g) as a yellow oil.

Step 2

To a solution of oxalyl chloride (2.9 ml, 33.3 mmol) in dichloromethane (50 ml) at −78° C. was added dry dimethyl sulfoxide (4.7 ml, 66.6 mmol) dropwise and the reaction mixture was stirred for 15 min. A solution of 1-benzyloxybutan-2-ol (4 g, 22.2 mmol) in dichloromethane (50 ml) was added. After 1 h, triethylamine (14 ml, 99.9 mmol) was added after 1 h the reaction mixture was warmed to room temperature. The reaction mixture was washed with water followed by brine. The organic layer was dried over MgSO₄, filtered and the solvent was evaporated to give 1-benzyloxybutan-2-one (3.9 g) as a yellow oil.

Step 3

1-Benzyloxypropan-2-one (4 g, 22.4 mmol, commercially available), NaCN (1.21 g, 25 mmol) and NH₄Cl (1.34 g, 25 mmol) were mixed in a 7N solution of NH₃ in methanol (13 ml, 0.12 mmol) and the reaction mixture was refluxed for 2 h. Additional 7N solution of NH₃ in methanol (13 ml) was added and refluxing was continued. After 2 h, the reaction mixture was cooled to room temperature and was diluted with 100 ml of dichloromethane. The resultant mixture was filtered, diluted again with another 100 ml of dichloromethane and was concentrated to give 2-amino-2-benzyloxymethylbutyronitrile (4 g) as a yellow oil.

Step 4

2-Amino-2-benzyloxymethylbutyronitrile (54.6 mg, 267 mmol) was added to a solution of 2-(RS)-(morpholine-4-carbonylamino]-3-trimethylsilanylpropionic acid (100 mg, 0.267 mmol) and HATU (122 mg, 0.320 mmol) in DMF (1 ml) and, followed by the addition of DIPEA (186 μl, 1.068 mmol). The reaction mixture was stirred at room temperature overnight and then diluted with 10 ml of ethyl acetate, washed with 5 ml of water and 5 ml of saturated solution of NaHCO₃ and dried over MgSO₄. The solvent was evaporated and the crude mixture was purified by HPLC to give the title compound. LCMS: 461.3(M+1)⁺, 483.2(M+Na)⁺, 459.1(M−1)⁻¹

### Example 11

### Synthesis of morpholine-4-carboxylic acid {1-(RS)-[(benzyloxymethylcyanomethylmethyl)-carbamoyl]-2-trimethylsilanylethyl}amide

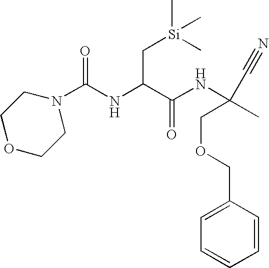 Step 1

1-Benzyloxypropan-2-one (5 g, 30 mmol, of commercially available), NaCN (1.64 g, 33.5 mmol) and NH₄Cl (1.79 g, 33.5 mmol) were mixed in a 2M solution of NH₃ in methanol (60 ml, 120 mmol) and the reaction mixture was refluxed for 2 h. Another 60 ml of 2M solution of NH₃ in methanol was added and refluxing was continued for another 2 h. The reaction mixture was cooled to room temperature and was diluted with 100 ml of dichloromethane. The resultant mixture was filtered, diluted again with another 100 ml of dichloromethane and concentrated to give 2-amino-3-benzyloxy-2-methylpropionitrile (5 g) as a yellow oil which was converted to the title compound as described in Example 10 above. LCMS: 447.6(+1)⁺¹, 469.4(M+Na)⁺, 445.4(M−1)⁻¹

### EXAMPLES

### Biological Examples

### Example 1

### Cathepsin B Assay

Solutions of test compounds in varying concentrations were prepared in 10 μL of dimethyl sulfoxide (DMSO) and then diluted into assay buffer (40 μL, comprising: N,N-bis(2-hydroxyethyl)-2-aminoethanesulfonic acid (BES), 50 mM (pH 6); polyoxyethylenesorbitan monolaurate, 0.05%; and dithiothreitol (DTT), 2.5 mM). Human cathepsin B (0.025 pMoles in 25 μL of assay buffer) was added to the dilutions. The assay solutions were mixed for 5-10 seconds on a shaker plate, covered and incubated for 30 minutes at room temperature. Z-FR-AMC (20 nMoles in 25 μL of assay buffer) was added to the assay solutions and hydrolysis was followed spectrophotometrically at (λ 460 nm) for 5 minutes. Apparent inhibition constants (Kᵢ) were calculated from the enzyme progress curves using standard mathematical models.

Compounds of the invention were tested by the above-described assay and observed to exhibit cathepsin B inhibitory activity.

### Example 2

### Cathepsin K Assay

Solutions of test compounds in varying concentrations were prepared in 10 μL of dimethyl sulfoxide (DMSO) and then diluted into assay buffer (40 μL, comprising: MES, 50 mM (pH 5.5); EDTA, 2.5 mM; and DTT, 2.5 mM). Human cathepsin K (0.0906 pMoles in 25 μL of assay buffer) was added to the dilutions. The assay solutions were mixed for 5-10 seconds on a shaker plate, covered and incubated for 30 minutes at room temperature. Z-Phe-Arg-AMC (4 nMoles in 25 μL of assay buffer) was added to the assay solutions and hydrolysis was followed spectrophotometrically at (, 460 nm) for 5 minutes. Apparent inhibition constants (Kᵢ) were calculated from the enzyme progress curves using standard mathematical models.

Compounds of the invention were tested by the above-described assay and observed to exhibit cathepsin K inhibitory activity.

### Example 3

### Cathepsin L Assay

Solutions of test compounds in varying concentrations were prepared in 10 μL of dimethyl sulfoxide (DMSO) and then diluted into assay buffer (40 μL, comprising: MES, 50 mM (pH 5.5); EDTA, 2.5 mM; and DTT, 2.5 mM). Human cathepsin L (0.05 pMoles in 25 μL of assay buffer) was added to the dilutions. The assay solutions were mixed for 5-10 seconds on a shaker plate, covered and incubated for 30 minutes at room temperature. Z-Phe-Arg-AMC (1 nMoles in 25 μL of assay buffer) was added to the assay solutions and hydrolysis was followed spectrophotometrically at (λ460 nm) for 5 minutes. Apparent inhibition constants (Kᵢ) were calculated from the enzyme progress curves using standard mathematical models.

Compounds of the invention were tested by the above-described assay and observed to exhibit cathepsin L inhibitory activity.

### Example 4

### Cathepsin S Assay

Solutions of test compounds in varying concentrations were prepared in 10 μL of dimethyl sulfoxide (DMSO) and then diluted into assay buffer (40 μL, comprising: MES, 50 mM (pH 6.5); EDTA, 2.5 mM; and NaCl, 100 mM); β-mercaptoethanol, 2.5 mM; and BSA, 0.001%. Human cathepsin S (0.05 pMoles in 25 mL of assay buffer) was added to the dilutions. The assay solutions were mixed for 5-10 seconds on a shaker plate, covered and incubated for 30 minutes at room temperature. Z-Val-Val-Arg-AMC (3 nMoles in 25 μL of assay buffer containing 10% DMSO) was added to the assay solutions and hydrolysis was followed spectrophotometrically (Ex: 355 nm, Em: 460 nm) for 5 minutes. Apparent inhibition constants (Kᵢ) were calculated from the enzyme progress curves using standard mathematical models.

Compounds of the invention were tested by the above-described assay and observed to exhibit cathepsin S inhibitory activity.

### Example 5

### Cathepsin F Assay

Solutions of test compounds in varying concentrations were prepared in 10 μL of dimethyl sulfoxide (DMSO) and then diluted into assay buffer (40 μL, comprising: MES, 50 mM (pH 6.5); EDTA, 2.5 mM; and NaCl, 100 mM); DTT, 2.5 mM; and BSA, 0.01%. Human cathepsin F (0.1 pMoles in 25 μL of assay buffer) was added to the dilutions. The assay solutions were mixed for 5-10 seconds on a shaker plate, covered and incubated for 30 minutes at room temperature. Z-Phe-Arg-AMC (2 nMoles in 25 μL of assay buffer containing 10% DMSO) was added to the assay solutions and hydrolysis was followed spectrophotometrically (at λ 460 nm) for 5 minutes. Apparent inhibition constants (Kᵢ) were calculated from the enzyme progress curves using standard mathematical models.

Compounds of the invention were tested by the above-described assay and observed to exhibit cathepsin F inhibitory activity.

### Example 6

### In Vito Iip10 Accumulation Assay

During normal antigen presentation, Iip10 is proteolytically degraded to enable loading of a peptide fragment and subsequent MHC-II presentation on the surface of antigen presenting cells. The cleavage process is mediated by Cathepsin S. Thus, the Iip10 assay is an in vitro measure of a compound's ability to block cathepsin S and by extension antigen presentation. A compound that causes the accumulation of Iip10 at low concentration would be expected to block presentation of antigens.

Method:

Raji cells (4×10⁶) were cultured with 0.02% DMSO or different concentrations of Cathepsin S inhibitors in RPMI medium 1640 containing 10% (v/v) FBS, 10 mM HEPES, 2 mM L-glutamine, and 1 mM sodium pyruvate for four hours at 37° C. in 5% CO₂ humidified atmosphere. After the culture period, cells were washed with cold PBS and cells were then lysed in NP-40 lysis buffer (5 mM EDTA, 1% NP-40, 150 mM NaCl, and 50 mM Tris, pH 7.6) with protease inhibitors. Protein determinations were performed and lysate samples were boiled in reducing SDS sample buffer. Proteins were separated by electrophoresis on 12% NuPAGE® Bis-Tris gels. Proteins were then transferred to nitrocellulose membranes, and after incubation with blocking buffer (5% non-fat dry milk in PBS-Tween), the blots were incubated with the primary antibody against human CD74 invariant chain synthetic peptide (1.5 to 2 μg/ml of mouse anti-CD74 monoclonal antibody, PIN.1, Stressgen Biotechnologies). Blots were then incubated with the secondary antibody, horseradish peroxidase conjugated donkey anti-mouse IgG, at a 1:10,000 dilution. Immunoreactive proteins were detected by chemiluminescence reaction using Pierce Super Signal® West Pico chemiluminescence substrate.

### Pharmaceutical Composition Examples

The following are representative pharmaceutical formulations containing a compound of the present invention.

### Tablet Formulation

The following ingredients are mixed intimately and pressed into single scored tablets.

| | Quantity per |
|---|---|
| Ingredient | tablet, mg |
| compound of this invention | 400 |
| cornstarch | 50 |
| croscarmellose sodium | 25 |
| lactose | 120 |
| magnesium stearate | 5 |

### Capsule Formulation

The following ingredients are mixed intimately and loaded into a hard-shell gelatin capsule.

| | Quantity per |
|---|---|
| Ingredient | capsule, mg |
| compound of this invention | 200 |
| lactose, spray-dried | 148 |
| magnesium stearate | 2 |

### Suspension Formulation

The following ingredients are mixed to form a suspension for oral administration.

| Ingredient | Amount | |
|---|---|---|
| compound of this invention | 1.0 | g |
| fumaric acid | 0.5 | g |
| sodium chloride | 2.0 | g |
| methyl paraben | 0.15 | g |
| propyl paraben | 0.05 | g |
| granulated sugar | 25.5 | g |
| sorbitol (70% solution) | 12.85 | g |
| Veegum K (Vanderbilt Co.) | 1.0 | g |
| flavoring | 0.035 | ml |
| colorings | 0.5 | mg |
| distilled water | q.s. to 100 | ml |

### Injectable Formulation

The following ingredients are mixed to form an injectable formulation.

| Ingredient | Amount |
|---|---|
| compound of this invention | 1.2 g |
| sodium acetate buffer solution, | 0.4 M 2.0 ml |
| HCl (1 N) or NaOH (1 N) | q.s. to suitable pH |
| water (distilled, sterile) | q.s.to 20 ml |

All of the above ingredients, except water, are combined and heated to 60-70° C. with stirring. A sufficient quantity of water at 60° C. is then added with vigorous stirring to emulsify the ingredients, and water then added q.s. to 100 g.

### Suppository Formulation

A suppository of total weight 2.5 g is prepared by mixing the compound of the invention with Witepsol® H-15 (triglycerides of saturated vegetable fatty acid; Riches-Nelson, Inc., New York), and has the following composition:

| | |
|---|---|
| compound of the invention | 500 mg |
| Witepsol ® H-15 | balance |

The foregoing invention has been described in some detail by way of illustration and example, for purposes of clarity and understanding. It will be obvious to one of skill in the art that changes and modifications may be practiced within the scope of the appended claims. Therefore, it is to be understood that the above description is intended to be illustrative and not restrictive. The scope of the invention should, therefore, be determined not with reference to the above description, but should instead be determined with reference to the following appended claims, along with the full scope of equivalents to which such claims are entitled. All patents, including Applicants' U.S. Provisional Application Ser. Nos. 60/540,581 and 60/547,498, filed on Jan. 30, 2004 and Feb. 24, 2004 and publications cited in this application are hereby incorporated by reference in their entirety for all purposes to the same extent as if each individual patent, patent application or publication were so individually denoted.

1. A compound of Formula (I): 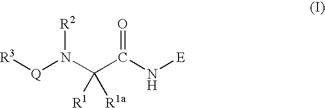
wherein:
Q is —CO—, —SO₂—, —OCO—, —NR⁴CO—, —NR⁴SO₂—, or —CHR— where R is haloalkyl and R⁴ is hydrogen, alkyl, hydroxyalkyl, alkoxyalkyl, or aralkyl;
E is:
(i) —C(R⁵)(R⁶)X¹ where X¹ is —C(R⁷)(R⁸)R¹⁰, —CH═CHS(O)₂R¹⁰, —C(R⁷)(R⁸)C(R⁷)(R⁸)OR¹⁰, —C(R⁷)(R⁸)CH₂OR¹⁰, —C(R⁷)(R⁸)CH₂N(R¹¹)SO₂R¹⁰, —C(R⁷)(R⁸)C(O)N(R¹¹)(CH₂)₂OR¹¹, —C(R⁷)(R⁸)C(O)NR¹⁰R¹¹ or —C(R⁷)(R⁸)C(O)N(R¹¹)(CH₂)₂NR¹⁰R¹¹;
(ii) —C(R^{5a})(R^{6a})CN;
where:
R⁵ and R^{5a} are independently hydrogen or alkyl;
R⁶ and R^{6a} are independently selected from the group consisting of hydrogen, alkyl, haloalkyl, cycloalkyl, cycloalkylalkyl, aryl, aralkyl, heteroaryl, heteroaralkyl, heterocycloalkyl, heterocycloalkylalkyl, -alkylene-X²—R¹² (where X² is —O—, —NR¹³—, —S(O)ₙ₁—, —CONR¹³—, —NR¹³CO—, —NR¹³C(O)O—, —NR¹³CONR¹³—, —OCONR¹³—, —NR¹³SO₂—, —SO₂NR¹³—, —NR¹³SO₂NR¹³—, —CO—, or —OC(O)— where n1 is 0-2 and each R¹³ is hydrogen or alkyl) and R¹² hydrogen, alkyl, haloalkyl, cycloalkyl, cycloalkylalkyl, heterocycloalkyl, heterocycloalkylalkyl, aryl, aralkyl, heteroaryl, or heteroaralkyl wherein the aromatic or alicyclic ring in R⁶ and R^{6a} is optionally substituted with one, two, or three R^{a} independently selected from alkyl, haloalkyl, alkoxy, hydroxy, haloalkoxy, halo, carboxy, alkoxycarbonyl, amino, monsubstituted amino, disubstituted amino, nitro, aryloxy, benzyloxy, acyl, alkylsulfonyl, or arylsulfonyl where the aromatic or alicyclic ring in R^{a} is optionally substituted with one or two substituents independently selected from alkyl, halo, alkoxy, haloalkyl, haloalkoxy, hydroxy, amino, alkylamino, dialkylamino, carboxy, or alkoxycarbonyl; or
R⁵ and R⁶ and R^{5a} and R^{6a} taken together with the carbon atom to which both R⁵ and R⁶ and R^{5a} and R^{6a} are attached form (i) cycloalkylene optionally substituted with one or two R^{b} independently selected from alkyl, halo, alkylamino, dialkylamino, aryl, aralkyl, cycloalkyl, cycloalkylalkyl, heteroaryl, heteroaralkyl, alkoxycarbonyl, or aryloxycarbonyl or (ii) heterocycloalkylene optionally substituted with one to four alkyl or one or two R^{c} independently selected from alkyl, haloalkyl, hydroxy, hydroxyalkyl, alkoxyalkyl, alkoxyalkyloxyalkyl, aryloxyalkyl, heteroaryloxyalkyl, aminoalkyl, acyl, aryl, aralkyl, heteroaryl, heteroaralkyl, heterocycloalkyl, heterocycloalkylalkyl, cycloalkyl, cycloalkylalkyl, —S(O)ₙ₂R¹⁴, -alkylene-S(O)ₙ₂—R¹⁵, —COOR¹⁶, -alkylene-COOR¹⁷, —CONR¹⁸R¹⁹, or -alkylene-CONR²⁰R²¹ (where n2 is 0-2 and R¹⁴-R¹⁷, R¹⁸ and R²⁰ are independently hydrogen, alkyl, haloalkyl, aryl, aralkyl, heteroaryl, heteroaralkyl, cycloalkyl, cycloalkylalkyl, or heterocycloalkyl and R¹⁹ and R²¹ are independently hydrogen or alkyl) wherein the aromatic or alicyclic ring in the groups attached to cycloalkylene or heterocycloalkylene is optionally substituted with one, two, or three substituents independently selected from alkyl, haloalkyl, cycloalkyl, cycloalkylalkyl, benzyl, alkoxy, hydroxy, haloalkoxy, halo, carboxy, alkoxycarbonyl, amino, monsubstituted amino, disubstituted amino, or acyl;
R⁷ is hydrogen or alkyl;
R⁸ is hydroxy; or
R⁷ and R⁸ together form oxo;
R¹⁰ is alkyl, aryl, aralkyl, heteroaryl, heteroaralkyl, cycloalkyl, cycloalkylalkyl, heterocycloalkyl, or heterocycloalkylalkyl wherein the aromatic or alicyclic ring in R¹⁰ is optionally substituted with one, two, or three R^{d} independently selected from alkyl, haloalkyl, alkoxy, alkoxyalkyl, cycloalkyl, hydroxy, haloalkoxy, halo, carboxy, alkoxycarbonyl, aminosulfonyl, alkylsulfonyl, arylsulfonyl, heteroarylsulfonyl, aryl, aralkyl, heteroaryl, amino, monsubstituted amino, disubstituted amino, carbamoyl, or acyl and wherein the aromatic or alicyclic ring in R^{d} is optionally substituted with one, two, or three substitutents independently selected from alkyl, haloalkyl, alkoxy, haloalkoxy, halo, hydroxy, carboxy, alkoxycarbonyl, amino, alkylamino, or dialkylamino; and
R¹¹ is hydrogen or alkyl; or
(iii) a group of formula (a): 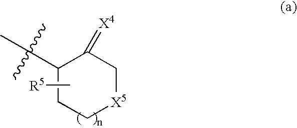
where:
n is 0, 1, or 2;
X⁴ is selected from —NR²²—, —S—, or —O— where R²² is hydrogen, alkyl, or alkoxy; and
X⁵ is —O—, —S—, —SO₂—, or —NR²³— where R²³ is selected from hydrogen, alkyl, haloalkyl, hydroxyalkyl, alkoxyalkyl, aryloxyalkyl, heteroaryloxyalkyl, aminoalkyl, acyl, aryl, aralkyl, heteroaryl, heteroaralkyl, cycloalkyl, cycloalkylalkyl, —S(O)₂R²⁴, -alkylene-S(O)ₙ₃—R²⁵, —COOR²⁶, -alkylene-COOR²⁷, —CONR²⁸R²⁹, or -alkylene-CONR³⁰R³¹ (where n3 is 0-2 and R²⁴-R²⁷, R²⁸ and R³⁰ are independently hydrogen, alkyl, haloalkyl, aryl, aralkyl, heteroaryl, heteroaralkyl, cycloalkyl, cycloalkylalkyl, heterocycloalkyl, or heterocycloalkylalkyl and R²⁹ and R³¹ are independently hydrogen or alkyl) where the aromatic or alicyclic ring in R²³ is optionally substituted with one, two, or three substituents independently selected from alkyl, haloalkyl, alkoxy, haloalkoxy, halo, hydroxy, amino, alkylamino, dialkylamino, carboxy, or alkoxycarbonyl and one substitutent selected from aryl, aralkyl, heteroaryl, or heteroaralkyl; and
R⁵ is as defined above;
R¹ is hydrogen or alkyl;
R^{1a} is 1,1-dialkylsilinan-4-ylalkylene or -(alkylene)-SiR³²R³³R³⁴ where R³² is alkyl, R³³ is alkyl, and R³⁴ is alkyl, alkenyl, cycloalkylalkyl, aryl, aralkyl, heteroaralkyl, or heterocycloalkylalkyl or R³³ and R³⁴ together with Si form a heterocycloalkylene ring containing the Si atom and 3 to 7 carbon ring atoms wherein one or two carbon ring atoms are optionally independently replaced with —NH—, —O—, —S—, —SO—, —SO₂—, —CO—, —CONH—, or —SO₂NH— and wherein the aralkyl, heteroaralkyl, heterocycloalkyl, or heterocycloalkylene ring in R^{1a} is optionally substituted on the ring with one, two, or three R^{e} independently selected from alkyl, haloalkyl, alkoxy, hydroxy, haloalkoxy, halo, carboxy, alkoxycarbonyl, amino, monsubstituted amino, disubstituted amino, nitro, aryloxy, benzyloxy, acyl, alkylsulfonyl, or arylsulfonyl and further wherein the aromatic or alicyclic ring in R^{e} is optionally substituted with one or two substituents independently selected from alkyl, halo, alkoxy, haloalkyl, haloalkoxy, hydroxy, amino, alkylamino, dialkylamino, carboxy, or alkoxycarbonyl;
R² is hydrogen or alkyl;
R³ is alkyl, haloalkyl, cycloalkyl, cycloalkylalkyl, aryl, aralkyl, heteroaryl, heteroaralkyl, heterocycloalkyl, heterocycloalkylalkyl, or -alkylene-X⁶—R³⁵ [wherein X⁶ is —NR³⁶—, —O—, —S(O)ₙ₄—, —CO—, —COO—, —OCO—, —NR³⁶CO—, —CONR³⁶—, —NR³⁶SO₂—, —SO₂NR³⁶—, —NR³⁶COO—, —OCONR³⁶—, —NR³⁶CONR³⁷—, or —NR³⁶SO₂NR³⁷— (where each R³⁶ and R³⁷ is independently hydrogen, alkyl, or acyl and n4 is 0-2) and R³⁵ is hydrogen, alkyl, haloalkyl, cycloalkyl, cycloalkylalkyl, heterocycloalkyl, heterocycloalkylalkyl, aryl, aralkyl, heteroaryl, or heteroaralkyl] wherein the alkylene chain in R³ is optionally substituted with one to four halo atoms and the aromatic and alicyclic rings in R³ are optionally substituted by one, two, or three R¹ independently selected from alkyl, aminoalkyl, halo, hydroxy, alkoxy, haloalkyl, haloalkoxy, oxo, cyano, nitro, acyl, acyloxy, aryl, aralkyl, heteroaryl, heteroaralkyl, cycloalkyl, cycloalkylalkyl, heterocycloalkyl, heterocycloalkylalkyl, aryloxy, benzyloxy, carboxy, alkoxycarbonyl, aryloxycarbonyl, carbamoyl, alkylthio, alkylsulfinyl, alkylsulfonyl, arylthio, arylsulfonyl, arylsulfinyl, alkoxycarbonylamino, aryloxycarbonylamino, alkylcarbamoyloxy, arylcarbamoyloxy, alkylsulfonylamino, arylsulfonylamino, aminosulfonyl, alkylaminosulfonyl, dialkylaminosulfonyl, arylaminosulfonyl, aralkylaminosulfonyl, aminocarbonyl, arylaminocarbonyl, aralkylaminocarbonyl, amino, monosubsituted or disubstituted amino, and further wherein the aromatic and alicyclic rings in R^{f} are optionally substituted with one, two, or three R⁹ wherein R⁹ is independently selected from alkyl, halo, haloalkyl, haloalkoxy, hydroxy, nitro, cyano, hydroxyalkyl, alkoxy, alkoxyalkyl, aminoalkyl, alkylthio, alkylsulfonyl, amino, monosubstituted amino, dialkylamino, aryl, heteroaryl, cycloalkyl, carboxy, carboxamido, or alkoxycarbonyl; or
a pharmaceutically acceptable salts thereof.

2. A compound of claim 1 wherein E is —CHR⁶C(O)R¹⁰ where R⁶ is alkyl and R¹⁰ is heteroaryl optionally substituted with one or two R^{d} independently selected from alkyl, haloalkyl, alkoxy, alkoxyalkyl, cycloalkyl, hydroxy, haloalkoxy, halo, carboxy, alkoxycarbonyl, aryl, heteroaryl, amino, monosubstituted amino, disubstituted amino, or acyl and wherein the aromatic or alicyclic ring in R^{d} is optionally substituted with one, two, or three substitutents independently selected from alkyl, haloalkyl, alkoxy, haloalkoxy, halo, hydroxy, carboxy, alkoxycarbonyl, amino, alkylamino, or dialkylamino.

3. A compound of claim 1 wherein E is —CR^{5a}R^{6a}CN, where R^{5a} and R^{6a} together with the carbon atom to which they are attached form cycloalkylene optionally substituted with one or two R^{b} independently selected from alkyl, halo, dialkylamino, aryl, aralkyl, cycloalkyl, cycloalkylalkyl, heteroaryl, heteroaralkyl, alkoxycarbonyl, or aryloxycarbonyl.

4. A compound of claim 1 wherein E is —CR^{5a}R^{6a}CN, where R^{5a} and R^{6a} together with the carbon atom to which they are attached form cyclopropyl.

5. A compound of claim 2 wherein R¹ and R² are hydrogen and Q is —CO—.

6. A compound of claim 2 wherein R^{1a} is -(alkylene)-SiR³²R³³R³⁴ where R³² is alkyl, R³³ is alkyl, and R³⁴ is alkyl or aralkyl.

7. (canceled)

8. A compound of claim 2 wherein R³ is heterocycloalkyl, aryl, or heteroaryl optionally substituted with one or two R^{f}.

9. A compound of claim 2 wherein R³ is morpholin-4-yl, 1-ethylpiperazin-4-yl, or phenyl optionally substituted with one or two substituents independently selected from halo, alkoxy, alkyl, haloalkoxy, phenyl, alkylsulfonyl, haloalkyl, heteroaryl, cyano, acyl, hydroxyalkyl, or alkoxycarbonyl.

10. A compound according to claim 1 selected from the group consisting of:
morpholine-4-carboxylic acid {1(R)-[1(S)-(benzoxazol-2-ylcarbonyl)-butylcarbamoyl]-2-trimethylsilanylethyl}amide;
morpholine-4-carboxylic acid {1(R)-[1(S)-(benzoxazol-2-ylcarbonyl)-propylcarbamoyl]-2-trimethylsilanylethyl}amide;
morpholine-4-carboxylic acid {1(R)-[1(R)-(benzoxazol-2-ylcarbonyl)-propylcarbamoyl]-2-trimethylsilanylethyl}amide;
morpholine-4-carboxylic acid {1(R)-[1(S)-(benzoxazol-2-ylcarbonyl)-pentylcarbamoyl]-2-trimethylsilanylethyl}amide;
morpholine-4-carboxylic acid {1 (R)-[1(S)-(5-chlorobenzoxazol-2-ylcarbonyl)-propylcarbamoyl]-2-trimethylsilanylethyl}amide;
morpholine-4-carboxylic acid {1(S)-[1(S)-(benzoxazol-2-ylcarbonyl)-propylcarbamoyl]-2-trimethylsilanylethyl}amide;
morpholine-4-carboxylic acid {1(R)-[1(S)-(benzoxazol-2-ylcarbonyl)-butylcarbamoyl]-2-trimethylsilanylethyl}amide;
1-(R)-morpholine-4-carboxylic acid [1-(1-cyanocyclopropylcarbamoyl)-2-(trimethylsilanyl)-ethyl]amide,
1-(R)-morpholine-4-carboxylic acid [1-(4-cyano-1-ethylpiperidin-4-ylcarbamoyl)-2-(trimethyl-silanyl)ethyl]amide;
1-(R)-morpholine-4-carboxylic acid [1-(4-cyano-1,1-dioxohexahydro-1λ⁶-thiopyran-4-yl-carbamoyl)-2-(trimethylsilanyl)ethyl]amide;
morpholine-4-carboxylic acid [1-(RS)-(1-benzyloxymethyl-1-cyanopropylcarbamoyl)-2-trimethyl-silanylethyl]-amide;
morpholine-4-carboxylic acid [1-(RS)-(2-benzyloxy-1-cyano-1-methyl-ethylcarbamoyl)-2-trimethylsilanylethyl]amide;
4-ethylpiperazine-1-carboxylic acid [1-(RS)-(1-cyanocyclopropylcarbamoyl)-2-trimethyl-silanylethyl]amide;
3′-methoxybiphenyl-3-carboxylic acid [1-(R)-(1-cyano-cyclopropylcarbamoyl)-2-trimethylsilanylethyl]amide;
N-[1-(RS)-(1-cyanocyclopropylcarbamoyl)-2-trimethylsilanylethyl]-3-iodobenzamide;
3′-trifluoromethoxybiphenyl-3-carboxylic acid [1-(RS)-(1-cyanocyclopropylcarbamoyl)-2-trimethylsilanylethyl]amide;
biphenyl-3-carboxylic acid [1-(RS)-(1-cyanocyclopropylcarbamoyl)-2-trimethylsilanylethyl]-amide;
2′,6′-dimethoxybiphenyl-3-carboxylic acid [1-(RS)-(1-cyanocyclopropylcarbamoyl)-2-trimethylsilanylethyl]amide;
4′-methylsulfonylbiphenyl-3-carboxylic acid [1-(RS)-(1-cyanocyclopropylcarbamoyl)-2-trimethylsilanylethyl]amide;
2′-chlorobiphenyl-3-carboxylic acid [1-(RS)-(1-cyanocyclopropylcarbamoyl)-2-trimethyl-silanylethyl]amide;
2′-trifluoromethylbiphenyl-3-carboxylic acid [1-(RS)-(1-cyanocyclopropylcarbamoyl)-2-trimethylsilanylethyl]amide;
3′-methylbiphenyl-3-carboxylic acid [1-(RS)-(1-cyanocyclopropylcarbamoyl)-2-trimethyl-silanylethyl]amide;
3′-trifluoromethoxybiphenyl-3-carboxylic acid [1-(RS)-(1-cyanocyclopropylcarbamoyl)-2-trimethylsilanylethyl]amide;
N-[1-(RS)-(1-cyanocyclopropylcarbamoyl)-2-trimethylsilanylethyl]-3-pyridin-3-ylbenzamide;
3′-cyanobiphenyl-3-carboxylic acid [1-(RS)-(1-cyanocyclopropylcarbamoyl)-2-trimethyl-silanylethyl]amide;
3′-hydroxymethylbiphenyl-3-carboxylic acid [1-(RS)-(1-cyanocyclopropylcarbamoyl)-2-trimethyl-silanylethyl]amide;
4′-hydroxymethylbiphenyl-3-carboxylic acid [1-(RS)-(1-cyanocyclopropylcarbamoyl)-2-trimethyl-silanylethyl]amide;
2′-methylbiphenyl-3-carboxylic acid [1-(RS)-(1-cyanocyclopropylcarbamoyl)-2-trimethyl-silanylethyl]amide;
3′-methoxycarbonylbiphenyl-3-carboxylic acid [1-(RS)-(1-cyanocyclopropylcarbamoyl)-2-trimethylsilanylethyl]amide;
4′-acetylbiphenyl-3-carboxylic acid [1-(RS)-(1-cyanocyclopropylcarbamoyl)-2-trimethyl-silanylethyl]amide;
3′-methoxybiphenyl-3-carboxylic acid [1-(RS)-(4-cyano-4-tetrahydrothiopyran-4-ylcarbamoyl)-2-trimethylsilanylethyl]amide;
3′-methoxybiphenyl-3-carboxylic acid [1-(RS)-(4-cyano-1,1-dioxohexahydro-1×6-thiopyran-4-yl-carbamoyl)-2-(trimethylsilanyl)ethyl]amide; and
1-[3-(benzyldimethylsilanyl)-2R-(2,2,2-trifluoro-1-phenylethylamino)propionyl]cyclopropane-carbonitrile;
or a pharmaceutically acceptable salt thereof.

11. A pharmaceutical composition comprising a compound of claim 1 and a pharmaceutically acceptable excipient.

12. (canceled)

14. The method of claim 18 wherein the cysteine protease is Cathepsin S.

15. The method of claim 14 wherein the disease is psoriasis, autoimmune disorder, allergic disorder, chronic obstructive pulmonary disease, or cardiovascular disease.

16-17. (canceled)

18. A method for treating a disease in an animal mediated by cysteine proteases, which method comprises administering to the animal a therapeutically effective amount of a compound of claim 1.

19. A compound of claim 3 wherein R¹ and R² are hydrogen and Q is —CO—.

20. A compound of claim 3 wherein R^{1a} is -(alkylene)-SiR³²R³³R³⁴ where R³² is alkyl, R³³ is alkyl, and R³⁴ is alkyl or aralkyl.

21. A compound of claim 3 wherein R³ is heterocycloalkyl, aryl, or heteroaryl optionally substituted with one or two R^{f}.

22. A compound of claim 3 wherein R³ is morpholin-4-yl, 1-ethylpiperazin-4-yl, or phenyl optionally substituted with one or two substituents independently selected from halo, alkoxy, alkyl, haloalkoxy, phenyl, alkylsulfonyl, haloalkyl, heteroaryl, cyano, acyl, hydroxyalkyl, or alkoxycarbonyl.

23. A compound of claim 4 wherein R¹ and R² are hydrogen and Q is —CO.

24. A compound of claim 4 wherein R^{1a} is -(alkylene)-SiR³²R³³R³⁴ where R³² is alkyl, R³³ is alkyl, and R³⁴ is alkyl or aralkyl.

25. A compound of claim 4 wherein R³ is heterocycloalkyl, aryl, or heteroaryl optionally substituted with one or two R^{f}.

26. A compound of claim 4 wherein R³ is morpholin-4-yl, 1-ethylpiperazin-4-yl, or phenyl optionally substituted with one or two substituents independently selected from halo, alkoxy, alkyl, haloalkoxy, phenyl, alkylsulfonyl, haloalkyl, heteroaryl, cyano, acyl, hydroxyalkyl, or alkoxycarbonyl.
